SEQUENCE LISTING (88 sequences)

SEQ ID NO: 1 - protein, 15 aa, synthetic construct
  REGION 1..15: note = AT-20.10 Light Chain CDR1
IASQSVDNYGISFMN

SEQ ID NO: 2 - protein, 7 aa, synthetic construct
  REGION 1..7: note = AT-20.10 Light Chain CDR2
AASNQGS

SEQ ID NO: 3 - protein, 9 aa, synthetic construct
  REGION 1..9: note = AT-20.10 Light Chain CDR3
QQSKEIPLT

SEQ ID NO: 4 - protein, 5 aa, synthetic construct
  REGION 1..5: note = AT-20.10 Heavy Chain CDR1
TQWMN

SEQ ID NO: 5 - protein, 17 aa, synthetic construct
  REGION 1..17: note = AT-20.10 Heavy Chain CDR2
QIFPASGSTYYNEMFKD

SEQ ID NO: 6 - protein, 8 aa, synthetic construct
  REGION 1..8: note = AT-20.10 Heavy Chain CDR3
RGGSGMDY

SEQ ID NO: 7 - protein, 111 aa, synthetic construct
  REGION 1..111: note = AT-20.10 Light Chain Variable
DIVLTQSPASLAVSLRHRATISCIASQSVDNYGISFMNWFQHKPGQPPKLLIYAASNQGSGVPARFSGSGSGTDFSLNIHPMEEDDTAMYFCQQSKEIPLTFGAGTKLELK

SEQ ID NO: 8 - protein, 117 aa, synthetic construct
  REGION 1..117: note = AT-20.10 Heavy Chain Variable
QVQLQQSGPDLVRPGTSVKISCKGSGYTFLTQWMNWVKQRPGQGLEWIGQIFPASGSTYYNEMFKDKATLTVDTSSSTAYMQLSSLTSEDTAVYFCARRGGSGMDYWGQGTSVTVSS

SEQ ID NO: 9 - protein, 15 aa, synthetic construct
  REGION 1..15: note = AT-20.10c Light Chain CDR1
IASQSVDNYGISFMN

SEQ ID NO: 10 - protein, 7 aa, synthetic construct
  REGION 1..7: note = AT-20.10c Light Chain CDR2
AASNQGS

SEQ ID NO: 11 - protein, 9 aa, synthetic construct
  REGION 1..9: note = AT-20.10c Light Chain CDR3
QQSKEIPLT

SEQ ID NO: 12 - protein, 5 aa, synthetic construct
  REGION 1..5: note = AT-20.10c Heavy Chain CDR1
TQWMN

SEQ ID NO: 13 - protein, 17 aa, synthetic construct
  REGION 1..17: note = AT-20.10c Heavy Chain CDR2
QIFPASGSTYYNEMFKD

SEQ ID NO: 14 - protein, 8 aa, synthetic construct
  REGION 1..8: note = AT-20.10c Heavy Chain CDR3
RGGSGMDY

SEQ ID NO: 15 - protein, 111 aa, synthetic construct; identical to SEQ ID NO: 7
  REGION 1..111: note = AT-20.10c Light Chain Variable

SEQ ID NO: 16 - protein, 117 aa, synthetic construct; identical to SEQ ID NO: 8
  REGION 1..117: note = AT-20.10c Heavy Chain Variable

SEQ ID NO: 17 - protein, 11 aa, synthetic construct
  REGION 1..11: note = AT-20.19 Light Chain CDR1
RASQGIRGNLD

SEQ ID NO: 18 - protein, 7 aa, synthetic construct
  REGION 1..7: note = AT-20.19 Light Chain CDR2
STSNLNS

SEQ ID NO: 19 - protein, 9 aa, synthetic construct
  REGION 1..9: note = AT-20.19 Light Chain CDR3
LQRNAYPLT

SEQ ID NO: 20 - protein, 5 aa, synthetic construct
  REGION 1..5: note = AT-20.19 Heavy Chain CDR1
DYHIN

SEQ ID NO: 21 - protein, 17 aa, synthetic construct
  REGION 1..17: note = AT-20.19 Heavy Chain CDR2
WIFPGSGSTYYNEKFKG

SEQ ID NO: 22 - protein, 11 aa, synthetic construct
  REGION 1..11: note = AT-20.19 Heavy Chain CDR3
GKSGYPFYFDY

SEQ ID NO: 23 - protein, 107 aa, synthetic construct
  REGION 1..107: note = AT-20.19 Light Chain Variable
DIQMIQSPSSMFASLGDRVSLSCRASQGIRGNLDWYQQKPGGTIKLLIYSTSNLNSGVPSRFSGSGSGSDYSLTISSLESEDFADYYCLQRNAYPLTFGAGTKLELR

SEQ ID NO: 24 - protein, 120 aa, synthetic construct
  REGION 1..120: note = AT-20.19 Heavy Chain Variable
QVQLQQSGRDLVKPGASVKISCKASGYTFTDYHINWVQQRPGQGLEWIGWIFPGSGSTYYNEKFKGKATLNVDKSSSTAYMLLSSLTSEDSAVYFCARGKSGYPFYFDYWGQGTTLTVSS

SEQ ID NO: 25 - protein, 11 aa, synthetic construct
  REGION 1..11: note = AT-20.19c Light Chain CDR1
RASQGIRGNLD

SEQ ID NO: 26 - protein, 7 aa, synthetic construct
  REGION 1..7: note = AT-20.19c Light Chain CDR2
STSNLNS

SEQ ID NO: 27 - protein, 9 aa, synthetic construct
  REGION 1..9: note = AT-20.19c Light Chain CDR3
LQRNAYPLT

SEQ ID NO: 28 - protein, 5 aa, synthetic construct
  REGION 1..5: note = AT-20.19c Heavy Chain CDR1
DYHIN

SEQ ID NO: 29 - protein, 17 aa, synthetic construct
  REGION 1..17: note = AT-20.19c Heavy Chain CDR2
WIFPGSGSTYYNEKFKG

SEQ ID NO: 30 - protein, 11 aa, synthetic construct
  REGION 1..11: note = AT-20.19c Heavy Chain CDR3
GKSGYPFYFDY

SEQ ID NO: 31 - protein, 107 aa, synthetic construct; identical to SEQ ID NO: 23
  REGION 1..107: note = AT-20.19c Light Chain Variable

SEQ ID NO: 32 - protein, 120 aa, synthetic construct; identical to SEQ ID NO: 24
  REGION 1..120: note = AT-20.19c Heavy Chain Variable

SEQ ID NO: 33 - protein, 11 aa, synthetic construct
  REGION 1..11: note = AT-20.19h Light Chain CDR1
RASQGIRGNLD

SEQ ID NO: 34 - protein, 7 aa, synthetic construct
  REGION 1..7: note = AT-20.19h Light Chain CDR2
STSNLNS

SEQ ID NO: 35 - protein, 9 aa, synthetic construct
  REGION 1..9: note = AT-20.19h Light Chain CDR3
LQRNAYPLT

SEQ ID NO: 36 - protein, 5 aa, synthetic construct
  REGION 1..5: note = AT-20.19h Heavy Chain CDR1
DYHIN

SEQ ID NO: 37 - protein, 17 aa, synthetic construct
  REGION 1..17: note = AT-20.19h Heavy Chain CDR2
WIFPGSGSTYYNEKFKG

SEQ ID NO: 38 - protein, 11 aa, synthetic construct
  REGION 1..11: note = AT-20.19h Heavy Chain CDR3
GKSGYPFYFDY

SEQ ID NO: 39 - protein, 107 aa, synthetic construct
  REGION 1..107: note = AT-20.19h Light Chain Variable
DIQMTQSPSSLSASVGDRVTITCRASQGIRGNLDWYQQKPGKAPKLLIYSTSNLNSGVPSRFSGSGSGSEYTLTISSLQPEDFATYYCLQRNAYPLTFGGGTKVEIK

SEQ ID NO: 40 - protein, 120 aa, synthetic construct
  REGION 1..120: note = AT-20.19h Heavy Chain Variable
QVQLVQSGAEVKKPGASVKVSCKASGYTFTDYHINWVRQATGQGLEWIGWIFPGSGSTYYNEKFKGRATLTVNKSISTAYMELSSLRSEDTAVYYCARGKSGYPFYFDYWGQGTLVTVSS

SEQ ID NO: 41 - protein, 11 aa, synthetic construct
  REGION 1..11: note = AT-20.25 Light Chain CDR1
KASENVGTFVS

SEQ ID NO: 42 - protein, 7 aa, synthetic construct
  REGION 1..7: note = AT-20.25 Light Chain CDR2
GASNRYT

SEQ ID NO: 43 - protein, 9 aa, synthetic construct
  REGION 1..9: note = AT-20.25 Light Chain CDR3
GQSYTYPYT

SEQ ID NO: 44 - protein, 5 aa, synthetic construct
  REGION 1..5: note = AT-20.25 Heavy Chain CDR1
SYWMH

SEQ ID NO: 45 - protein, 17 aa, synthetic construct
  REGION 1..17: note = AT-20.25 Heavy Chain CDR2
VIHPNSGSINYNEKFKS

SEQ ID NO: 46 - protein, 10 aa, synthetic construct
  REGION 1..10: note = AT-20.25 Heavy Chain CDR3
GNSLYYALDY

SEQ ID NO: 47 - protein, 107 aa, synthetic construct
  REGION 1..107: note = AT-20.25 Light Chain Variable
NIVMTQSPKSMSMSVGERVTLSCKASENVGTFVSWYQQKPEQSPKLLIFGASNRYTGVPDRFTGSGSATDFTLIISSVQAEDLADYHCGQSYTYPYTFGGGTKLEIK

SEQ ID NO: 48 - protein, 119 aa, synthetic construct
  REGION 1..119: note = AT-20.25 Heavy Chain Variable
QVQLQQPGAELVKPGASVKLSCKASGYTFTSYWMHWVKQRPGQGLEWLGVIHPNSGSINYNEKFKSKATLTVDKSSSTAYMQLSSLTSEDSAVYYCAGGNSLYYALDYWGQGTSVTVSS

SEQ ID NO: 49 - protein, 11 aa, synthetic construct
  REGION 1..11: note = AT-20.25h Light Chain CDR1
KASENVGTFVS

SEQ ID NO: 50 - protein, 7 aa, synthetic construct
  REGION 1..7: note = AT-20.25h Light Chain CDR2
GASNRYT

SEQ ID NO: 51 - protein, 9 aa, synthetic construct
  REGION 1..9: note = AT-20.25h Light Chain CDR3
GQSYTYPYT

SEQ ID NO: 52 - protein, 5 aa, synthetic construct
  REGION 1..5: note = AT-20.25h Heavy Chain CDR1
SYWMH

SEQ ID NO: 53 - protein, 17 aa, synthetic construct
  REGION 1..17: note = AT-20.25h Heavy Chain CDR2
VIHPNSGSINYNEKFKS

SEQ ID NO: 54 - protein, 10 aa, synthetic construct
  REGION 1..10: note = AT-20.25h Heavy Chain CDR3
GNSLYYALDY

SEQ ID NO: 55 - protein, 107 aa, synthetic construct
  REGION 1..107: note = AT-20.25h Light Chain Variable
EIVMTQSPATLSLSPGERVTLSCKASENVGTFVSWYQQKPGQAPRLLIFGASNRYTGIPARFSGSGSATDFTLTISSLQPEDFAVYYCGQSYTYPYTFGQGTKLEIK

SEQ ID NO: 56 - protein, 119 aa, synthetic construct
  REGION 1..119: note = AT-20.25h Heavy Chain Variable
QVQLVQSGAEVKKPGASVKVSCKASGYTFTSYWMHWVRQAPGQGLEWLGVIHPNSGSINYNEKFKSRATLTVDKSTSTAYMELSSLRSEDTAVYYCAGGNSLYYALDYWGQGTLVTVSS

SEQ ID NO: 57 - protein, 11 aa, synthetic construct
  REGION 1..11: note = AT-20.25hvA Light Chain CDR1
KASENVGTFVS

SEQ ID NO: 58 - protein, 7 aa, synthetic construct
  REGION 1..7: note = AT-20.25hvA Light Chain CDR2
GASNRYT

SEQ ID NO: 59 - protein, 9 aa, synthetic construct
  REGION 1..9: note = AT-20.25hvA Light Chain CDR3
GQSYTYPYT

SEQ ID NO: 60 - protein, 5 aa, synthetic construct
  REGION 1..5: note = AT-20.25hvA Heavy Chain CDR1
SYWMH

SEQ ID NO: 61 - protein, 17 aa, synthetic construct
  REGION 1..17: note = AT-20.25hvA Heavy Chain CDR2
VIHPNSGSINYNEKFKS

SEQ ID NO: 62 - protein, 10 aa, synthetic construct
  REGION 1..10: note = AT-20.25hvA Heavy Chain CDR3
GNSLYYALDY

SEQ ID NO: 63 - protein, 107 aa, synthetic construct
  REGION 1..107: note = AT-20.25hvA Light Chain Variable
EIVMTQSPATLSLSPGERVTLSCKASENVGTFVSWYQQKPGQAPRLLIFGASNRYTGIPARFSGSGSGTDFTLTISSLQPEDFAVYYCGQSYTYPYTFGQGTKLEIK

SEQ ID NO: 64 - protein, 119 aa, synthetic construct
  REGION 1..119: note = AT-20.25hvA Heavy Chain Variable
QVQLVQSGAEVKKPGASVKVSCKASGYTFTSYWMHWVRQAPGQGLEWLGVIHPNSGSINYNEKFKSRATLTVDKSTSTAYMELSSLRSEDTAVYYCARGNSLYYALDYWGQGTLVTVSS

SEQ ID NO: 65 - protein, 11 aa, synthetic construct
  REGION 1..11: note = AT-20.25hvG Light Chain CDR1
KASENVGTFVS

SEQ ID NO: 66 - protein, 7 aa, synthetic construct
  REGION 1..7: note = AT-20.25hvG Light Chain CDR2
GASNRYT

SEQ ID NO: 67 - protein, 9 aa, synthetic construct
  REGION 1..9: note = AT-20.25hvG Light Chain CDR3
QQSYTYPYT

SEQ ID NO: 68 - protein, 5 aa, synthetic construct
  REGION 1..5: note = AT-20.25hvG Heavy Chain CDR1
SYWMH

SEQ ID NO: 69 - protein, 17 aa, synthetic construct
  REGION 1..17: note = AT-20.25hvG Heavy Chain CDR2
VIHPNSGSINYNEKFKS

SEQ ID NO: 70 - protein, 10 aa, synthetic construct
  REGION 1..10: note = AT-20.25hvG Heavy Chain CDR3
GNSLYYALDY

SEQ ID NO: 71 - protein, 107 aa, synthetic construct
  REGION 1..107: note = AT-20.25hvG Light Chain Variable
EIVMTQSPATLSLSPGERVTLSCKASENVGTFVSWYQQKPGQAPRLLIFGASNRYTGIPARFSGSGSATDFTLTISSLQPEDFAVYYCQQSYTYPYTFGQGTKLEIK

SEQ ID NO: 72 - protein, 119 aa, synthetic construct; identical to SEQ ID NO: 64
  REGION 1..119: note = AT-20.25hvG Heavy Chain Variable

SEQ ID NO: 73 - protein, 11 aa, synthetic construct
  REGION 1..11: note = AT-20.25hvAG Light Chain CDR1
KASENVGTFVS

SEQ ID NO: 74 - protein, 7 aa, synthetic construct
  REGION 1..7: note = AT-20.25hvAG Light Chain CDR2
GASNRYT

SEQ ID NO: 75 - protein, 9 aa, synthetic construct
  REGION 1..9: note = AT-20.25hvAG Light Chain CDR3
QQSYTYPYT

SEQ ID NO: 76 - protein, 5 aa, synthetic construct
  REGION 1..5: note = AT-20.25hvAG Heavy Chain CDR1
SYWMH

SEQ ID NO: 77 - protein, 17 aa, synthetic construct
  REGION 1..17: note = AT-20.25hvAG Heavy Chain CDR2
VIHPNSGSINYNEKFKS

SEQ ID NO: 78 - protein, 10 aa, synthetic construct
  REGION 1..10: note = AT-20.25hvAG Heavy Chain CDR3
GNSLYYALDY

SEQ ID NO: 79 - protein, 107 aa, synthetic construct
  REGION 1..107: note = AT-20.25hvAG Light Chain Variable
EIVMTQSPATLSLSPGERVTLSCKASENVGTFVSWYQQKPGQAPRLLIFGASNRYTGIPARFSGSGSGTDFTLTISSLQPEDFAVYYCQQSYTYPYTFGQGTKLEIK

SEQ ID NO: 80 - protein, 119 aa, synthetic construct; identical to SEQ ID NO: 64
  REGION 1..119: note = AT-20.25hvAG Heavy Chain Variable

SEQ ID NO: 81 - DNA, 333 bp, synthetic construct
  source 1..333: mol_type = other DNA
  misc_feature 1..333: note = AT-20.10 Light Chain Variable
gacatagtcctgacacagagccctgcgagcctggccgtgagcctggggcagagagccaccatcagctgcagagctagcgagagcgtggacaactacggcatcagcttcatgaactggtttcagcagaagcccgggcagccccccaagctgcttatctatgcagctagcaaccaaggcagcggcgttcccgctagattcagcggcagcggcagcggcaccgacttcagcctgaacatccaccccatggaggaggacgacaccgccatgtacttctgtcagcagagcaaggagatccccctgaccttcggcgccggcaccaagctggagctgaag

SEQ ID NO: 82 - DNA, 351 bp, synthetic construct
  source 1..351: mol_type = other DNA
  misc_feature 1..351: note = AT-20.10 Heavy Chain Variable
caagtgcagctgcagcaaagcggtcctgacctggtgagacccggtacaagcgtgaagatcagctgcaagggcagcggctacaccttcctgacacagtggatgaactgggtgaagcagagacccggccaaggcctggagtggatcgggcagatcttccccgctagcggcagcacctactacaacgagatgttcaaggacaaggccaccctgaccgtggacacaagcagcagcaccgcctacatgcagctgagcagcctgacaagcgaggacaccgccgtgtacttctgcgctagaagaggcggcagcggcatggactactggggccaaggcacaagcgtgaccgtgagcagc

SEQ ID NO: 83 - DNA, 318 bp, synthetic construct
  source 1..318: mol_type = other DNA
  misc_feature 1..318: note = AT-20.19 Light Chain Variable
gacattcagatgattcagagccctagcagcatgttcgctagcctgggcgacagagtgagcctgagctgcagagctagccaaggcatcagaggcaacctggactggtatcagcagaagcccggcggcaccatcaagctgctgatctacagcacaagcaacctgaacagcggcgtgcctagcagattcagcggcagcggcagcggcagcgactacagcctgaccatcagcagcctggagagcgaggacttcgccgactactactgcctgcagagaaacgcctaccccctgaccttcggcgccggcaccaagctggagctg

SEQ ID NO: 84 - DNA, 360 bp, synthetic construct
  source 1..360: mol_type = other DNA
  misc_feature 1..360: note = AT-20.19 Heavy Chain Variable
caagtgcagctgcagcagagcggcagagacctggtgaagcccggcgctagcgtgaagatcagctgcaaggctagcggctacaccttcaccgactaccacatcaactgggtgcagcagagacccggccaaggcctggagtggatcggctggatcttccccggcagcggcagcacctactacaacgagaagttcaagggcaaggccaccctgaacgtggacaagagcagcagcaccgcctacatgctgctgagcagcctgacaagcgaggacagcgccgtgtacttctgcgctagaggcaagagcggctaccccttctacttcgactactggggccaaggcaccaccctgaccgtgagcagc

SEQ ID NO: 85 - DNA, 321 bp, synthetic construct
  source 1..321: mol_type = other DNA
  misc_feature 1..321: note = AT-20.25 Light Chain Variable
aacatagttatgactcagtctccgaaatccatgtccatgtctgttggggaaagagtcacgctcagttgtaaagccagtgagaacgtgggaacttttgtgtcctggtaccagcaaaagcccgaacagtctccgaaacttttgatctttggggccagcaatagatacaccggggtccccgatcgtttcacgggttctggttctgccacagactttactctcatcattagttctgtacaagctgaagatttggccgactatcactgtggtcagtcttacacatatccctacactttcggtggcggaacaaaactggaaattaaa

SEQ ID NO: 86 - DNA, 357 bp, synthetic construct
  source 1..357: mol_type = other DNA
  misc_feature 1..357: note = AT-20.25 Heavy Chain Variable
caagttcaactacaacaacccggggctgaactggtaaaacctggagcttctgtaaaactgtcctgtaaggcaagtggttatacatttacttcatattggatgcattgggtgaaacaacgtccaggtcaaggactggaatggcttggtgtgattcaccccaattctggatccataaattacaacgaaaagtttaaatctaaagctactctcacggttgacaaatcctcaagtacggcatatatgcaactctcttctttgacttccgaggacagcgcagtctactactgtgccggcgggaattctctgtactatgccttggactattggggacaaggcacaagcgtgaccgtgagctct

SEQ ID NO: 87 - DNA, 321 bp, synthetic construct
  source 1..321: mol_type = other DNA
  misc_feature 1..321: note = AT-20.25h Light Chain Variable
gagattgtaatgacccaaagtccagcaaccttgtctttgtcacctggggagcgggtaactttgtcatgtaaggcttcagagaatgtcggaaccttcgtaagctggtaccaacagaaacccggacaagctccacgcctgttgatcttcggcgcttccaatcggtacacagggattcctgcacgcttctcagggtccggttccgctaccgattttactctaactatatctagcctacagcctgaagattttgccgtctactactgtgggcaatcatatacatacccatatacgttcggccaaggcaccaaacttgagatcaaa

SEQ ID NO: 88 - DNA, 357 bp, synthetic construct
  source 1..357: mol_type = other DNA
  misc_feature 1..357: note = AT-20.25h Heavy Chain Variable
caagtacaactagtccaatccggcgcagaagtgaagaaacccggcgcttccgtcaaagtgagctgtaaggcttcagggtacacattcacctcatattggatgcactgggttaggcaggctcctggacagggcctcgagtggcttggagtgatacacccaaactcaggctccatcaactataacgaaaagtttaaatcccgtgctactctaacagtggacaagagtacaagcactgcttacatggaattgtcaagtctgcgaagcgaagacactgctgtttattactgtgcaggtgggaattccctgtattacgctcttgactattggggccagggtacactggtcaccgtctctagc

(19) United States
(12) Patent Application Publication
(10) Pub. No.: US 2026/0115303 A1
(43) Pub. Date: Apr. 30, 2026

(54) CDH17-BINDING ANTIBODIES AND METHODS OF USE THEREOF

(71) Applicant: Antharis Therapeutics Inc., San Diego, CA (US)

(72) Inventors: Raphael Ribeiro-Pinaud, San Diego, CA (US); Sai Siddhant Suraparaju, San Diego, CA (US); Jinming Xia, San Diego, CA (US); Eric Sjoberg, San Diego, CA (US); Jerome Han, San Diego, CA (US); Mary Dwyer, San Diego, CA (US); Daniel Yazbeck, San Diego, CA (US)

(21) Appl. No.: 19/368,730

(22) Filed: Oct. 24, 2025

Related U.S. Application Data
(60) Provisional application No. 63/712,336, filed on Oct. 25, 2024.
(60) Provisional application No. 63/782,509, filed on Apr. 2, 2025.
(60) Provisional application No. 63/838,533, filed on Jul. 3, 2025.

(51) Int. Cl. A61K 47/68; A61K 9/00; A61K 39/00; A61K 45/06; A61P 35/00; C07K 16/28

(52) U.S. Cl. CPC A61K 47/68031; A61K 9/0019; A61K 45/06; A61K 47/68037; A61K 47/6849; A61K 47/6889; A61P 35/00; C07K 16/28; A61K 2039/54; A61K 2039/545; C07K 2317/24; C07K 2317/33; C07K 2317/34; C07K 2317/55; C07K 2317/565; C07K 2317/622; C07K 2317/732; C07K 2317/92

(57) ABSTRACT

Provided herein are, inter alia, novel antibodies that bind to cadherin-17 (CDH17) thereby effectively targeting cells expressing CHD17. The antibodies provided herein may be used, inter alia, for therapeutic cancer applications, including, in some embodiments, treatment of multiple cancer types, which may include gastrointestinal cancer, colorectal cancer, stomach cancer, pancreas cancer, neuroendocrine cancer, or ovarian cancer.

### CROSS-REFERENCE TO RELATED APPLICATIONS

This application claims the benefit of priority to U.S. Provisional Patent Application Ser. No. 63/712,336, filed Oct. 25, 2024, U.S. Provisional Patent Application Ser. No. 63/782,509, filed Apr. 2, 2025, and U.S. Provisional Patent Application Ser. No. 63/838,533, filed Jul. 3, 2025, which are hereby incorporated by reference in their entireties.

### REFERENCE TO A “SEQUENCE LISTING,” A TABLE, OR A COMPUTER PROGRAM LISTING APPENDIX SUBMITTED ON A COMPACT DISK

The Sequence Listing written in file 2025-07-03Sequence_Listing_ST26057868-511P03US.xml, created on Jul. 3, 2025, 118,784 bytes, machine format IBM-PC, MS-Windows operating system, is hereby incorporated by reference in its entirety for all purposes.

### BACKGROUND

Gastrointestinal (GI) tract cancers are common in the United States and worldwide, and are among the leading causes of cancer-related mortality. Although some types of GI cancers have the possibility to be treated if detected in their early stages, many are diagnosed at advanced stages when mestastatis has occurred. This limits the effectiveness of traditional treatments and approaches. Additionally, many GI cancers have the tendency to develop resistance to chemotherapy. The five-year survival rates for certain types of GI cancers such as pancreatic cancer and gastric cancer remain as low as around 5-15% and 35% respectively if the cancer is regional. In recent times, monoclonal antibodies (mAbs) have come forward as promising therapeutic approaches for various cancers, including GI cancers. mAbs can be designed to target tumor-specific antigens, or antigens that are overexpressed on such tumors.

Cadherin-17 (CDH17) is an antigen that is overexpressed on many different types of GI cancers. In a comprehensive immunohistochemistry study of 18132 tumors, Jacobsen et al. have identified that the highest rate of positive staining of CDH17 is in colorectal neoplasms including those of neuroendocrine origin (50%-100%), other gastrointestinal (GI) adenocarcinomas (42.7%-61.6%), mucinous ovarian cancer (61.1%), acinar cell carcinoma of the pancreas (28.6%), adenocarcinomas of the cervix uteri (52.6%), and pancreato-biliary adenocarcinomas (40.5%-69.8%) (doi.org/10.1016/j.prp.2024.155175). This makes CDH17 a relevant and promising target antigen for monoclonal antibodies with the goal of treating GI cancers. Provided herein, inter alia, are compositions and methods to address these and other problems in the art.

### BRIEF SUMMARY

In an aspect is provided an anti-cadherin-17 (CDH17) antibody including a light chain variable domain and a heavy chain variable domain, wherein the light chain variable domain includes: a CDR L1, a CDR L2, and a CDR L3 as set forth in Table 1; and wherein the heavy chain variable domain includes: a CDR H1, a CDR H2, and a CDR H3 as set forth in Table 1.

In another aspect is provided an anti-cadherin-17 (CDH17) antibody, wherein the anti-CDH17 antibody binds the same epitope as an antibody including: a light chain variable region domain including a CDR L1, a CDR L2, and a CDR L3 as set forth in Table 1 and a heavy chain variable domain including a CDR H1, a CDR H2, and a CDR H3 as set forth in Table 1.

In another aspect is provided an isolated nucleic acid encoding the antibody provided herein including embodiments thereof.

In another aspect is provided a cell including the isolated nucleic acid provided herein including embodiments thereof.

In another aspect is provided a pharmaceutical composition including a therapeutically effective amount of an the antibody provided herein including embodiments thereof or a therapeutically effective amount of the isolated nucleic acid provided herein including embodiments thereof; and a pharmaceutically acceptable excipient.

In another aspect is provided a method of treating a subject in need thereof, the method including administering to a subject a therapeutically effective amount of the antibody provided herein including embodiments thereof.

In another aspect is provided the use of the antibody provided herein including embodiments thereof in the manufacture of a medicament for the treatment of a tumor or cancer.

### BRIEF DESCRIPTION OF THE DRAWINGS

FIGS. 1A-1E present illustrative data showing high affinity cell surface binding of the murine AT-20 antibodies across multiple CDH17 expressing tumor cell lines, in accordance with embodiments.

FIGS. 2A-2D present illustrative data showing high affinity cell surface binding of the chimeric AT-20 antibodies across multiple CDH17 expressing tumor cell lines, in accordance with embodiments.

FIGS. 3A-3B present illustrative data showing cell surface binding of the chimeric AT-20 antibodies to Expi293 cells transfected with a human CDH17 expression plasmid and Expi293 cells transfected with a cynomolgus CDH17 expression plasmid, in accordance with embodiments.

FIGS. 4A-4E present illustrative data showing high affinity cell surface binding of a humanized AT-20.25 antibody, a humanized AT-20.25 antibody conjugated to monomethyl auristatin E (MMAE) via a cleavable linker, and a humanized AT-20.25 antibody conjugated to Deruxtecan (GGFG-DXd) across multiple CDH17 expressing tumor cell lines, in accordance with embodiments.

FIGS. 5A-5B present illustrative data showing high affinity cell surface binding of the humanized AT-20.25 antibody to Expi293 cells transfected with a human CDH17 expression plasmid, and Expi293 cells transfected with a cynomolgus CDH17 expression plasmid, in accordance with embodiments. FIGS. 5C-5D present illustrative data showing internalization of humanized AT-20.25 antibody by SNU-16 and HPAF2 human tumor cell lines, in accordance with embodiments.

FIGS. 6A-6D present illustrative data showing ADC effects of chimeric AT-20 family antibodies bound by a secondary antibody conjugated to monomethyl auristatin E (MMAE) across multiple CDH17 expressing tumor cell lines, in accordance with embodiments.

FIGS. 7A-7D present illustrative data showing ADC effects of chimeric AT-20 family antibodies bound by a secondary antibody conjugated to DX8951 across multiple CDH17 expressing tumor cell lines, in accordance with embodiments.

FIGS. 8A-8E present experimental data showing ADC effects of a humanized AT-20.25 antibody directly conjugated to monomethyl auristatin E (MMAE) across multiple CDH17 expressing tumor cell lines, in accordance with embodiments.

FIGS. 9A-9E present experimental data showing ADC effects of the humanized AT-20.25 antibody directly conjugated to Deruxtecan (GGFG-DXd) across multiple CDH17 expressing tumor cell lines, in accordance with embodiments.

FIGS. 10A-10C present experimental data showing effects on in vivo tumor volume following treatment with the humanized AT-20.25 antibody directly conjugated to monomethyl aunstatin E (MMAE) via a cleavable linker in SNU-16 gastric tumors in mice, in accordance with embodiments. FIG. 10A shows effects on SNU-16 in vivo tumor volume at various dosages of AT-20.25h ADC during treatment with two injection times (day 0 and day 20). FIG. 10B shows effects of AT-20.25h antibody drug conjugates comprising DXd or MMAE payload on SNU-16 in vivo tumor volume over time after a single injection, in accordance with embodiments. FIG. 10C shows effects of AT-20.25h antibody drug conjugates comprising DXd or MMAE payload on SW403 colorectal cancer in vivo tumor volume over time with two injections, in accordance with embodiments. FIG. 10D shows effects of the AT-20.25h ADC on a large SNU-16 tumor and a small SNU-16 tumor after a single injection, in accordance with embodiments.

FIG. 11A presents experimental data showing effects on in vivo tumor volume following a single injection of a humanized AT-20.25 antibody directly conjugated to monomethyl auristatin E (MMAE) via a cleavable linker in AsPC-1 pancreatic tumors in mice, in accordance with embodiments. FIG. 11B presents experimental data showing effects on in vivo tumor volume following a single injection of a humanized AT-20.25 antibody directly conjugated to monomethyl auristatin E (MMAE) or DXd via a cleavable linker in HPAF-2 pancreatic tumors in mice, in accordance with embodiments.

FIGS. 12A-12D present experimental enzyme-linked immunosorbent assay (ELISA) data showing CDH17 domain specificity of binding by chimeric AT-20.10, chimeric AT-20.19, humanized AT-20.25, and TORL 07-0653-h43 anti-CDH17 antibody respectively, in accordance with embodiments.

FIG. 13A presents experimental data showing cross-reactivity of the humanized AT-20.25 antibody (AT20.25h) against recombinant P-cadherin, E-cadherin, and cadherin-16 (CDH16) as compared to reactivity with recombinant human and recombinant non-human primate cadherin-17 (CDH17) by ELISA, in accordance with embodiments. FIG. 13B presents experimental data showing ELISA binding assay results of humanized AT-20.25 antibody (AT20.25h) to human CDH17, cynomolgus CDH17, Rat CDH17, and mouse CDH 17 over a range of antibody concentrations, in accordance with embodiments.

FIG. 14 presents experimental data showing Surface Plasmon Resonance (SPR) analysis of the humanized AT-20.25 antibody binding to various concentrations of recombinant human CDH17 to obtain a calculated equilibrium dissociation constant (KD), in accordance with embodiments.

FIG. 15A and FIG. 15B present experimental data showing antibody-dependent cellular cytotoxicity (ADCC) results for treatment of SNU-16 cells in vitro with humanized AT-20.25 antibodies and human peripheral blood mononuclear cells (PBMCs), in accordance with embodiments.

FIGS. 16A-D present experimental data showing levels of cytokine release by peripheral blood mononuclear cells after treatment with humanized AT-20.25h antibody, in accordance with embodiments.

FIGS. 17A and 17B present experimental data showing levels of binding of humanized AT-20.25h antibody to polyspecificity reagents (PSRs) and to baculovirus particles (BVP), respectively, in accordance with embodiments.

FIGS. 18A and 18B present experimental data showing stability of humanized AT-20.25 antibody in human and cynomolgus blood plasma, respectively, in accordance with embodiments.

FIG. 18C presents experimental data showing thermal stability of humanized AT-20.25 antibody in vitro, in accordance with embodiments.

FIG. 19 presents experimental data showing effects on in vivo tumor volume following a single injection of a humanized AT-20.25 antibody directly conjugated to monomethyl auristatin E (MMAE) or DXd via a cleavable linker in CR5055 colorectal patient-derived tumors (PDX) in mice, in accordance with embodiments.

FIG. 20 shows an example of a treatment paradigm for subjects in need thereof, such as subjects having or at risk of having a tumor or cancer, in accordance with embodiments.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., cadherin-17) in other proteins with different numbering systems. For example, by performing a simple sequence alignment with a protein (e.g., cadherin-17) the identity and location of residues corresponding to specific positions of the protein are identified in other protein sequences aligning to the protein. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three-dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the inventions disclosed herein. Some embodiments include a conservatively modified variant of a CDR (e.g., a CDR L1, a CDR L2, a CDR L3, a CDR H1, a CDR H2, or a CDR H3) herein. Some embodiments include a conservatively modified variant of a variable domain (e.g., VH or VL) herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab2, monovalent IgGs, scFv, scFv-Fc, minibodies, IgNAR, V-NAR, hcIgG, or VhH. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “light chain” is used according to its ordinary meaning in the biological arts, and refers to the polypeptide formed by a light chain variable domain (VL) and a light chain constant domain (CL). Likewise, the term “heavy chain” is used according to its ordinary meaning in the biological arts, and refers to the polypeptide formed by a heavy chain variable domain (VH) and one or more heavy chain constant domains (CH1, CH2, CH3). The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable domains of the heavy (VH) and light chain (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (e.g, variable region including domain VH and VL) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

A “therapeutic agent” as referred to herein, is a composition useful in treating or preventing a disease such as cancer (e.g., gastrointestinal cancer). In embodiments, the therpaeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The phrase “specifically (or selectively) binds” to an antibody or “specifically (or selectively) immunoreactive with,” when referring to a protein or peptide (e.g., CDH17), refers to a binding reaction that is determinative of the presence of the protein (e.g., CDH17), often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “Cadherin-17” or “CDH17”, is used herein according to its plain ordinary meaning and refers to a calcium-dependent, membrane-associated glycoprotein of the cadherin superfamily, also known as Liver-Intestine Cadherin (LI-Cadherin). In embodiments, the CDH17 is a type 1 transmembrane protein containing 7 cadherin domains, a transmembrane region, and one cytoplasmic domain. In embodiments, the CDH17 mediates calcium-dependent cell adhesion.

The term “cadherin-17” or “CDH17” as used herein refers to any recombinant or naturally-occurring forms of cadherin-17 (CDH17) or variants or homologs thereof that maintain CDH17 activity (e.g. within at least 50%, 80%, 90%, 95%, 96%, 97%, 98%, 99% or 100% activity compared to CDH17). In embodiments, the variants or homologs have at least 90%, 95%, 96%, 97%, 98%, 99% or 100% amino acid sequence identity across the whole sequence or a portion of the sequence (e.g. a 10, 20, 50, 100, 150 or 200 continuous amino acid portion) compared to a naturally occurring CDH17 polypeptide. In embodiments, CDH17 is substantially identical to the protein identified by the UniProt reference number Q12864 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “stem cell” as provided herein refers to a cell characterized by the ability of self-renewal through mitotic cell division and the potential to differentiate into a tissue or an organ. Among mammalian stem cells, embryonic stem cells (ES cells) and somatic stem cells (e.g., hematopoietic stem cells (HSC)) can be distinguished. Embryonic stem cells reside in the blastocyst and give rise to embryonic tissues, whereas somatic stem cells reside in adult tissues for the purpose of tissue regeneration and repair.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre-B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly, an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. CDH17) relative to the activity or function of the protein in the absence of the inhibitor. In embodiments inhibition means negatively affecting (e.g. decreasing) the concentration or levels of a protein (e.g. CDH17 protein) relative to the concentration or level of the protein in the absence of the inhibitor. In embodiments inhibition refers to reduction of a disease or symptoms of disease. In embodiments, inhibition refers to a reduction in the activity of a protein (e.g. CDH17 protein). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. CDH17 protein). In embodiments, inhibition refers to a reduction of activity of a protein (e.g. CDH17 protein) resulting from a direct interaction (e.g. an inhibitor binds to the protein). In embodiments, inhibition refers to a reduction of activity of a protein (e.g. CDH17 protein) from an indirect interaction (e.g. an inhibitor binds to a protein that activates the protein, thereby preventing target protein activation).

Thus, the terms “inhibitor,” “repressor” or “antagonist” or “downregulator” interchangeably refer to a substance capable of detectably decreasing the expression or activity of a given gene or protein (e.g. CDH17). The antagonist can decrease expression or activity of a protein (e.g. CDH17 protein) by 10%, 20%, 30%, 40%, 50%, 60%, 70%, 80%, 90% or more in comparison to a control in the absence of the antagonist. In certain instances, a protein's (e.g. CDH17 protein) expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high-performance liquid chromatography (HPLC). A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, colorectal cancer, gastrointestinal cancer, kidney cancer, leukemia, lung cancer (e.g., non-small cell lung cancer), melanoma, ovarian cancer, prostate cancer, renal cancer, pancreatic cancer, brain cancer (e.g., glioblastomas and/or astrocytomas), liver cancer, gastric cancer or a sarcoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. In some cases, cell migration (e.g., increased cell migration relative to a control cell) can be indicative of a risk of metastasis or a tendency to metastasize in a tumor tissue or can potentiate metastasis, cancer tissue, tumor cell, or cancer cell. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., cancer (e.g. gastrointestinal cancer, colorectal cancer)) means that the disease (e.g., cancer (e.g. gastrointestinal cancer, colorectal cancer)) is caused by (in whole or in part), or a symptom of the disease is caused by (in whole or in part) the substance or substance activity or function. Alternatively, the substance (e.g., CDH17) may be an indicator of the disease (e.g., cancer (e.g. gastrointestinal cancer, colorectal cancer)). Thus, an associated substance may serve as a means of targeting disease tissue (e.g., cancer cells (e.g., gastrointestinal cancer cells, colorectal cancer cells)).

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., gastrointestinal cancer, colorectal cancer). In embodiments, the therapeutic agent is a antineoplastic agent. In embodiments, the therpaeutic agent is an anti-cancer agent. “Anti-cancer agent” and “antineoplastic agent” are used in accordance with their plain ordinary meaning and refer to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. In embodiments, the antineoplastic agent is a chemotherapeutic. In embodiments, the antineoplastic agent is an agent identified herein having utility in methods of treating cancer. In embodiments, the antineoplastic agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus, in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Antibody Compositions

Provided herein are, inter alia, antibodies (e.g., humanized antibodies, monoclonal antibodies) and antibody compositions (e.g., scFvs) that are capable of binding cadherin-17 (CDH17). The antibodies and antibody compositions provided herein include novel light and heavy chain domain CDRs and framework regions, and bind CDH17 with high efficiency and specificity, thereby effectively targeting these target proteins and/or cells expressing the target proteins. The light and heavy chain domains of the antibodies provided herein may form part of recombinant proteins also referred to herein as antibody compositions (e.g., IgG (for instance, IgG1) or scFv) to be used, inter alia, as cancer therapeutics and for diagnostic purposes.

Thus, in an aspect is provided an anti-cadherin-17 (CDH17) antibody including a light chain variable domain and a heavy chain variable domain, wherein the light chain variable domain includes: a CDR L1, a CDR L2, and a CDR L3 as set forth in Table 1; and wherein the heavy chain variable domain includes: a CDR H1, a CDR H2, and a CDR H3 as set forth in Table 1.

In embodiments, the light chain variable domain includes an amino acid sequence with at least 80%, 85%, 90%, 91%, 92%, 93%, 94%, 95%, 96%, 97%, 98%, 99%, or 100% sequence identity with a light chain variable domain sequence as set forth in Table 1. In embodiments, the light chain variable domain includes an amino acid sequence with at least 80% sequence identity with a light chain variable domain sequence as set forth in Table 1. In embodiments, the light chain variable domain includes an amino acid sequence with at least 85% sequence identity with a light chain variable domain sequence as set forth in Table 1. In embodiments, the light chain variable domain includes an amino acid sequence with at least 90% sequence identity with a light chain variable domain sequence as set forth in Table 1. In embodiments, the light chain variable domain includes an amino acid sequence with at least 91% sequence identity with a light chain variable domain sequence as set forth in Table 1. In embodiments, the light chain variable domain includes an amino acid sequence with at least 92% sequence identity with a light chain variable domain sequence as set forth in Table 1. In embodiments, the light chain variable domain includes an amino acid sequence with at least 93% sequence identity with a light chain variable domain sequence as set forth in Table 1. In embodiments, the light chain variable domain includes an amino acid sequence with at least 94% sequence identity with a light chain variable domain sequence as set forth in Table 1. In embodiments, the light chain variable domain includes an amino acid sequence with at least 95% sequence identity with a light chain variable domain sequence as set forth in Table 1. In embodiments, the light chain variable domain includes an amino acid sequence with at least 96% sequence identity with a light chain variable domain sequence as set forth in Table 1. In embodiments, the light chain variable domain includes an amino acid sequence with at least 97% sequence identity with a light chain variable domain sequence as set forth in Table 1. In embodiments, the light chain variable domain includes an amino acid sequence with at least 98% sequence identity with a light chain variable domain sequence as set forth in Table 1. In embodiments, the light chain variable domain includes an amino acid sequence with at least 99% sequence identity with a light chain variable domain sequence as set forth in Table 1. In embodiments, the light chain variable domain includes an amino acid sequence with at least 100% sequence identity with a light chain variable domain sequence as set forth in Table 1.

In embodiments, the light chain variable domain is an amino acid sequence with at least 80%, 85%, 90%, 91%, 92%, 93%, 94%, 95%, 96%, 97%, 98%, 99%, or 100% sequence identity with a light chain variable domain sequence as set forth in Table 1. In embodiments, the light chain variable domain is an amino acid sequence with at least 80% sequence identity with a light chain variable domain sequence as set forth in Table 1. In embodiments, the light chain variable domain is an amino acid sequence with at least 85% sequence identity with a light chain variable domain sequence as set forth in Table 1. In embodiments, the light chain variable domain is an amino acid sequence with at least 90% sequence identity with a light chain variable domain sequence as set forth in Table 1. In embodiments, the light chain variable domain is an amino acid sequence with at least 91% sequence identity with a light chain variable domain sequence as set forth in Table 1. In embodiments, the light chain variable domain is an amino acid sequence with at least 92% sequence identity with a light chain variable domain sequence as set forth in Table 1. In embodiments, the light chain variable domain is an amino acid sequence with at least 93% sequence identity with a light chain variable domain sequence as set forth in Table 1. In embodiments, the light chain variable domain is an amino acid sequence with at least 94% sequence identity with a light chain variable domain sequence as set forth in Table 1. In embodiments, the light chain variable domain is an amino acid sequence with at least 95% sequence identity with a light chain variable domain sequence as set forth in Table 1. In embodiments, the light chain variable domain is an amino acid sequence with at least 96% sequence identity with a light chain variable domain sequence as set forth in Table 1. In embodiments, the light chain variable domain is an amino acid sequence with at least 97% sequence identity with a light chain variable domain sequence as set forth in Table 1. In embodiments, the light chain variable domain is an amino acid sequence with at least 98% sequence identity with a light chain variable domain sequence as set forth in Table 1. In embodiments, the light chain variable domain is an amino acid sequence with at least 99% sequence identity with a light chain variable domain sequence as set forth in Table 1. In embodiments, the light chain variable domain is an amino acid sequence with at least 100% sequence identity with a light chain variable domain sequence as set forth in Table 1.

In embodiments, the heavy chain variable domain includes an amino acid sequence with at least 80%, 85%, 90%, 91%, 92%, 93%, 94%, 95%, 96%, 97%, 98%, 99%, or 100% sequence identity with heavy chain variable domain sequence as set forth in Table 1. In embodiments, the heavy chain variable domain includes an amino acid sequence with at least 80% sequence identity with heavy chain variable domain sequence as set forth in Table 1. In embodiments, the heavy chain variable domain includes an amino acid sequence with at least 85% sequence identity with heavy chain variable domain sequence as set forth in Table 1. In embodiments, the heavy chain variable domain includes an amino acid sequence with at least 90% sequence identity with heavy chain variable domain sequence as set forth in Table 1. In embodiments, the heavy chain variable domain includes an amino acid sequence with at least 91% sequence identity with heavy chain variable domain sequence as set forth in Table 1. In embodiments, the heavy chain variable domain includes an amino acid sequence with at least 92% sequence identity with heavy chain variable domain sequence as set forth in Table 1. In embodiments, the heavy chain variable domain includes an amino acid sequence with at least 93% sequence identity with heavy chain variable domain sequence as set forth in Table 1. In embodiments, the heavy chain variable domain includes an amino acid sequence with at least 94% sequence identity with heavy chain variable domain sequence as set forth in Table 1. In embodiments, the heavy chain variable domain includes an amino acid sequence with at least 95% sequence identity with heavy chain variable domain sequence as set forth in Table 1. In embodiments, the heavy chain variable domain includes an amino acid sequence with at least 96% sequence identity with heavy chain variable domain sequence as set forth in Table 1. In embodiments, the heavy chain variable domain includes an amino acid sequence with at least 97% sequence identity with heavy chain variable domain sequence as set forth in Table 1. In embodiments, the heavy chain variable domain includes an amino acid sequence with at least 98% sequence identity with heavy chain variable domain sequence as set forth in Table 1. In embodiments, the heavy chain variable domain includes an amino acid sequence with at least 99% sequence identity with heavy chain variable domain sequence as set forth in Table 1. In embodiments, the heavy chain variable domain includes an amino acid sequence with at least 100% sequence identity with heavy chain variable domain sequence as set forth in Table 1.

In embodiments, the heavy chain variable domain is an amino acid sequence with at least 80%, 85%, 90%, 91%, 92%, 93%, 94%, 95%, 96%, 97%, 98%, 99%, or 100% sequence identity with heavy chain variable domain sequence as set forth in Table 1. In embodiments, the heavy chain variable domain is an amino acid sequence with at least 80% sequence identity with heavy chain variable domain sequence as set forth in Table 1. In embodiments, the heavy chain variable domain is an amino acid sequence with at least 85% sequence identity with heavy chain variable domain sequence as set forth in Table 1. In embodiments, the heavy chain variable domain is an amino acid sequence with at least 90% sequence identity with heavy chain variable domain sequence as set forth in Table 1. In embodiments, the heavy chain variable domain is an amino acid sequence with at least 91% sequence identity with heavy chain variable domain sequence as set forth in Table 1. In embodiments, the heavy chain variable domain is an amino acid sequence with at least 92% sequence identity with heavy chain variable domain sequence as set forth in Table 1. In embodiments, the heavy chain variable domain is an amino acid sequence with at least 93% sequence identity with heavy chain variable domain sequence as set forth in Table 1. In embodiments, the heavy chain variable domain is an amino acid sequence with at least 94% sequence identity with heavy chain variable domain sequence as set forth in Table 1. In embodiments, the heavy chain variable domain is an amino acid sequence with at least 95% sequence identity with heavy chain variable domain sequence as set forth in Table 1. In embodiments, the heavy chain variable domain is an amino acid sequence with at least 96% sequence identity with heavy chain variable domain sequence as set forth in Table 1. In embodiments, the heavy chain variable domain is an amino acid sequence with at least 97% sequence identity with heavy chain variable domain sequence as set forth in Table 1. In embodiments, the heavy chain variable domain is an amino acid sequence with at least 98% sequence identity with heavy chain variable domain sequence as set forth in Table 1. In embodiments, the heavy chain variable domain is an amino acid sequence with at least 99% sequence identity with heavy chain variable domain sequence as set forth in Table 1. In embodiments, the heavy chain variable domain is an amino acid sequence with at least 100% sequence identity with heavy chain variable domain sequence as set forth in Table 1.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO: 1, a CDR L2 amino acid sequence as set forth in SEQ ID NO:2, and a CDR L3 amino acid sequence as set forth in SEQ ID NO:3; and wherein the heavy chain variable domain includes: a CDR H1 amino acid sequence as set forth in SEQ ID NO:4, a CDR H2 amino acid sequence as set forth in SEQ ID NO:5, and a CDR H3 amino acid sequence as set forth in SEQ ID NO:6. In embodiments, the anti-CDH17 antibody includes a light chain variable domain including the amino acid sequence as set forth in SEQ ID NO:7 and a heavy chain variable domain including the amino acid sequence as set forth in SEQ ID NO: 8. In embodiments, the anti-CDH17 antibody includes a light chain variable domain having the amino acid sequence as set forth in SEQ ID NO:7 and a heavy chain variable domain having the amino acid sequence as set forth in SEQ ID NO:8. In embodiments, the anti-CDH17 antibody is referred to herein as clone AT-20.10.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:9, a CDR L2 amino acid sequence as set forth in SEQ ID NO: 10, and a CDR L3 amino acid sequence as set forth in SEQ ID NO:11; and wherein the heavy chain variable domain includes: a CDR H1 amino acid sequence as set forth in SEQ ID NO: 12, a CDR H2 amino acid sequence as set forth in SEQ ID NO:13, and a CDR H3 amino acid sequence as set forth in SEQ ID NO: 14. In embodiments, the anti-CDH17 antibody includes a light chain variable domain including the amino acid sequence as set forth in SEQ ID NO: 15 and a heavy chain variable domain including the amino acid sequence as set forth in SEQ ID NO: 16. In embodiments, the anti-CDH17 antibody includes a light chain variable domain having the amino acid sequence as set forth in SEQ ID NO: 15 and a heavy chain variable domain having the amino acid sequence as set forth in SEQ ID NO: 16. In embodiments, the anti-CDH17 antibody is referred to herein as clone AT-20.10c.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO: 17, a CDR L2 amino acid sequence as set forth in SEQ ID NO: 18, and a CDR L3 amino acid sequence as set forth in SEQ ID NO: 19; and wherein the heavy chain variable domain includes: a CDR H1 amino acid sequence as set forth in SEQ ID NO:20, a CDR H2 amino acid sequence as set forth in SEQ ID NO:21, and a CDR H3 amino acid sequence as set forth in SEQ ID NO:22. In embodiments, the anti-CDH17 antibody includes a light chain variable domain including the amino acid sequence as set forth in SEQ ID NO:23 and a heavy chain variable domain including the amino acid sequence as set forth in SEQ ID NO: 24. In embodiments, the anti-CDH17 antibody includes a light chain variable domain having the amino acid sequence as set forth in SEQ ID NO:23 and a heavy chain variable domain having the amino acid sequence as set forth in SEQ ID NO:24. In embodiments, the anti-CDH17 antibody is referred to herein as clone AT-20.19.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:25, a CDR L2 amino acid sequence as set forth in SEQ ID NO:26, and a CDR L3 amino acid sequence as set forth in SEQ ID NO:27; and wherein the heavy chain variable domain includes: a CDR H1 amino acid sequence as set forth in SEQ ID NO:28, a CDR H2 amino acid sequence as set forth in SEQ ID NO:29, and a CDR H3 amino acid sequence as set forth in SEQ ID NO:30. In embodiments, the anti-CDH17 antibody includes a light chain variable domain including the amino acid sequence as set forth in SEQ ID NO:31 and a heavy chain variable domain including the amino acid sequence as set forth in SEQ ID NO:32. In embodiments, the anti-CDH17 antibody includes a light chain variable domain having the amino acid sequence as set forth in SEQ ID NO:31 and a heavy chain variable domain having the amino acid sequence as set forth in SEQ ID NO:32. In embodiments, the anti-CDH17 antibody is referred to herein as clone AT-20.19c.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:33, a CDR L2 amino acid sequence as set forth in SEQ ID NO:34, and a CDR L3 amino acid sequence as set forth in SEQ ID NO:35; and wherein the heavy chain variable domain includes: a CDR H1 amino acid sequence as set forth in SEQ ID NO:36, a CDR H2 amino acid sequence as set forth in SEQ ID NO:37, and a CDR H3 amino acid sequence as set forth in SEQ ID NO:38. In embodiments, the anti-CDH17 antibody includes a light chain variable domain including the amino acid sequence as set forth in SEQ ID NO:39 and a heavy chain variable domain including the amino acid sequence as set forth in SEQ ID NO:40. In embodiments, the anti-CDH17 antibody includes a light chain variable domain having the amino acid sequence as set forth in SEQ ID NO:39 and a heavy chain variable domain having the amino acid sequence as set forth in SEQ ID NO:40. In embodiments, the anti-CDH17 antibody is referred to herein as clone AT-20.19h.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:41, a CDR L2 amino acid sequence as set forth in SEQ ID NO:42, and a CDR L3 amino acid sequence as set forth in SEQ ID NO:43; and wherein the heavy chain variable domain includes: a CDR H1 amino acid sequence as set forth in SEQ ID NO:44, a CDR H2 amino acid sequence as set forth in SEQ ID NO:45, and a CDR H3 amino acid sequence as set forth in SEQ ID NO:46. In embodiments, the anti-CDH17 antibody includes a light chain variable domain including the amino acid sequence as set forth in SEQ ID NO:47 and a heavy chain variable domain including the amino acid sequence as set forth in SEQ ID NO:48. In embodiments, the anti-CDH17 antibody includes a light chain variable domain having the amino acid sequence as set forth in SEQ ID NO:47 and a heavy chain variable domain having the amino acid sequence as set forth in SEQ ID NO:48. In embodiments, the anti-CDH17 antibody is referred to herein as clone AT-20.25.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:49, a CDR L2 amino acid sequence as set forth in SEQ ID NO:50, and a CDR L3 amino acid sequence as set forth in SEQ ID NO:51; and wherein the heavy chain variable domain includes: a CDR H1 amino acid sequence as set forth in SEQ ID NO:52, a CDR H2 amino acid sequence as set forth in SEQ ID NO:53, and a CDR H3 amino acid sequence as set forth in SEQ ID NO:54. In embodiments, the anti-CDH17 antibody includes a light chain variable domain including the amino acid sequence as set forth in SEQ ID NO:55 and a heavy chain variable domain including the amino acid sequence as set forth in SEQ ID NO:56. In embodiments, the anti-CDH17 antibody includes a light chain variable domain having the amino acid sequence as set forth in SEQ ID NO:55 and a heavy chain variable domain having the amino acid sequence as set forth in SEQ ID NO:56. In embodiments, the anti-CDH17 antibody is referred to herein as clone AT-20.25h.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:57, a CDR L2 amino acid sequence as set forth in SEQ ID NO:58, and a CDR L3 amino acid sequence as set forth in SEQ ID NO:59; and wherein the heavy chain variable domain includes: a CDR H1 amino acid sequence as set forth in SEQ ID NO:60, a CDR H2 amino acid sequence as set forth in SEQ ID NO:61, and a CDR H3 amino acid sequence as set forth in SEQ ID NO:62. In embodiments, the anti-CDH17 antibody includes a light chain variable domain including the amino acid sequence as set forth in SEQ ID NO:63 and a heavy chain variable domain including the amino acid sequence as set forth in SEQ ID NO:64. In embodiments, the anti-CDH17 antibody includes a light chain variable domain having the amino acid sequence as set forth in SEQ ID NO:63 and a heavy chain variable domain having the amino acid sequence as set forth in SEQ ID NO:64. In embodiments, the anti-CDH17 antibody is referred to herein as clone AT-20.25hvA.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:65, a CDR L2 amino acid sequence as set forth in SEQ ID NO:66, and a CDR L3 amino acid sequence as set forth in SEQ ID NO:67; and wherein the heavy chain variable domain includes: a CDR H1 amino acid sequence as set forth in SEQ ID NO:68, a CDR H2 amino acid sequence as set forth in SEQ ID NO:69, and a CDR H3 amino acid sequence as set forth in SEQ ID NO:70. In embodiments, the anti-CDH17 antibody includes a light chain variable domain including the amino acid sequence as set forth in SEQ ID NO:71 and a heavy chain variable domain including the amino acid sequence as set forth in SEQ ID NO:72. In embodiments, the anti-CDH17 antibody includes a light chain variable domain having the amino acid sequence as set forth in SEQ ID NO:71 and a heavy chain variable domain having the amino acid sequence as set forth in SEQ ID NO:72. In embodiments, the anti-CDH17 antibody is referred to herein as clone AT-20.25hvG.

In embodiments, the anti-cadherin-17 (CDH17) antibody includes a light chain variable domain and a heavy chain variable domain, wherein the light chain variable domain includes: a CDR L1 amino acid sequence as set forth in SEQ ID NO:73, a CDR L2 amino acid sequence as set forth in SEQ ID NO:74, and a CDR L3 amino acid sequence as set forth in SEQ ID NO:75; and wherein the heavy chain variable domain includes: a CDR H1 amino acid sequence as set forth in SEQ ID NO:76, a CDR H2 amino acid sequence as set forth in SEQ ID NO:77, and a CDR H3 amino acid sequence as set forth in SEQ ID NO:78. In embodiments, the anti-CDH17 antibody includes a light chain variable domain including the amino acid sequence as set forth in SEQ ID NO:79 and a heavy chain variable domain including the amino acid sequence as set forth in SEQ ID NO:80. In embodiments, the anti-CDH17 antibody includes a light chain variable domain having the amino acid sequence as set forth in SEQ ID NO:79 and a heavy chain variable domain having the amino acid sequence as set forth in SEQ ID NO:80. In embodiments, the anti-CDH17 antibody is referred to herein as clone AT-20.25hvAG.

In embodiments, the antibody is a humanized antibody. In embodiments, the antibody is a chimeric antibody. In embodiments, the antibody is an IgG. In embodiments, the antibody is an IgG1. In embodiments, the antibody is a Fab′ fragment. In embodiments, the antibody is a single chain antibody (scFv). In embodiments, the light chain variable domain and the heavy chain variable domain form part of a scFv

In embodiments, the antibody is capable of binding cadherin-17 (CDH17). In embodiments, the antibody is bound to CDH17. In embodiments, CDH17 forms part of a cell. In embodiments, the cell is a cancer cell. In embodiments, the cancer cell is a gastrointestinal cancer cell, a colorectal cancer cell, a stomach cancer cell, a pancreas cancer cell, a neuroendocrine cancer cell, or an ovarian cancer cell. In embodiments, the cancer cell is a gastrointestinal cancer cell. In embodiments, the cancer cell is a colorectal cancer cell. In embodiments, the cancer cell is a stomach cancer cell. In embodiments, the cancer cell is a pancreas cancer cell. In embodiments, the cancer cell is a neuroendocrine cancer cell. In embodiments, the cancer cell is an ovarian cancer cell.

In another aspect is provided an anti-cadherin-17 (CDH17) antibody, wherein the anti-CDH17 antibody binds the same epitope as an antibody including: a light chain variable region domain including a CDR L1, a CDR L2, and a CDR L3 as set forth in Table 1 and a heavy chain variable domain including a CDR H1, a CDR H2, and a CDR H3 as set forth in Table 1.

In embodiments, the anti-CDH17 antibody provided herein including embodiments thereof can be used as an antibody-drug conjugate (ADC) and act as a vehicle to deliver a toxic payload such as auristatins, maytansinoids, tubulysins, A payload of an anti-CDH17 ADC (e.g., an AT-20 family antibody, such as AT-20.25h, conjugated to a payload, e.g., via a linker) can be an antineoplastic agent. Topoisomerase I inhibitors and other DNA targeting payloads which are released once the antibody is internalized by the tumor cell. In embodiments, the toxic payload is an auristatin, a maytansinoid, a tubulin inhibitor, a tubulysin, a Topoisomerase I inhibitor, or a DNA-targeting payload. In embodiments, the toxic payload is an auristatin. In embodiments, the toxic payload is a maytansinoid. In embodiments, the toxic payload is a tubulin inhibitor, such as monomethyl auristatin E (MMAE). In embodiments, the toxic payload is a tubulysin. In embodiments, the toxic payload is a topoisomerase inhibitor, such as belotecan. In embodiments, the toxic payload is a Topoisomerase I inhibitor, such as DX8951 or DXd. In embodiments, the payload can be DX8951, which can have anti-tumor or anti-cancer effects on cancer and tumor cells. In some cases, anti-tumor or anti-cancer effects of DX8951 payloads can include anti-proliferation effects on cancer or tumor cells and/or reduction in tumor or cancer growth rates. In embodiments, the payload can be DXd, which can have anti-tumor or anti-cancer effects on cancer and tumor cells. In some cases, anti-tumor or anti-cancer effects of DXd payloads can include anti-proliferation effects on cancer or tumor cells and/or reduction in tumor or cancer growth rates. In embodiments, the toxic payload is a DNA-targeting payload. In embodiments, the payload can be monomethyl auristatin E (MMAE), which can have anti-tumor or anti-cancer effects on cancer and tumor cells. In some cases, anti-tumor or anti-cancer effects of MMAE payloads can include anti-proliferation effects on cancer or tumor cells and/or reduction in tumor or cancer growth rates. In embodiments, the payload can be belotecan, which can have anti-tumor anti-cancer effects on cancer and tumor cells. In some cases, anti-tumor or anti-cancer effects of belotecan payloads can include anti-proliferation effects on cancer or tumor cells and/or reduction in tumor or cancer growth rates. The intracellular delivery of the toxic payload can result in inhibition of cell proliferation and ultimately in the death of the cells. In embodiments, the payload is conjugated to the anti-CDH17 antibody provided herein including embodiments thereof via a cleavable linker or non-cleavable linker. In embodiments, the payload is conjugated to the anti-CDH17 antibody provided herein including embodiments thereof via a cleavable linker. In embodiments, the payload is conjugated to the anti-CDH17 antibody provided herein including embodiments thereof via a non-cleavable linker. In embodiments, a linker conjugated to the anti-CDH17 antibody (e.g., AT-20 family antibody described herein, such as AT-20.25h) can be a tetrapeptide linker, such as GGFG. For example, an ADC described herein can comprise an anti-CDH17 antibody described herein (e.g., AT-20.25h) conjugated to a payload (e.g., DX8951, DXd, or MMAE) by a GGFG linker. For example, an ADC can comprise an AT-20 family antibody (e.g., AT-20.25h) conjugated to a GGFG-DX8951 drug-linker conjugate. In some embodiments, an ADC can comprise an AT-20 family antibody (e.g., AT-20.25h) conjugated to a GGFG-DXd drug-linker conjugate. In some cases, an ADC described herein can comprise an AT-20 family antibody (e.g., AT-20.25h) conjugated to a GGFG-MMAE drug-linker conjugate. In embodiments, a linker conjugated to the anti-CDH17 antibody (e.g., AT-20 family antibody described herein, such as AT-20.25h) can be an MC-Val-Cit-PAB linker. For example, an ADC described herein can comprise an anti-CDH17 antibody described herein (e.g., AT-20.25h) conjugated to a payload (e.g., DX8951, DXd, or MMAE) by a MC-Val-Cit-PAB linker (e.g., an AT-20 family antibody conjugated to MC-Val-Cit-PAB-Exatecan). In some cases, an anti-CDH17 antibody described herein can be conjugated to a drug-linker conjugate, such as Deruxtecan (BroadPharm, CAS-1599440-13-7) or MC-Val-Cit-PAB-Exatecan (BroadPharm Catalog No. BP-41119). In some cases, an ADC described herein can comprise an anti-CDH17 antibody described herein (e.g., AT-20.25h) conjugated to an MC-Val-Cit-PAB-DX8951 drug-linker conjugate. In some cases, an ADC described herein can comprise an anti-CDH17 antibody described herein (e.g., AT-20.25h) conjugated to an MC-Val-Cit-PAB-DXd drug-linker conjugate. In some cases, an ADC described herein can comprise an anti-CDH17 antibody described herein (e.g., AT-20.25h) conjugated to a MC-Val-Cit-PAB-MMAE drug-linker conjugate.

### Nucleic Acid Compositions

The compositions provided herein include nucleic acid molecules encoding anti-CDH17 antibodies and recombinant proteins provided herein including embodiments thereof. The anti-CDH17 antibodies encoded by the isolated nucleic acids provided herein are described in detail throughout this application (including the description above and in the examples section). Thus, in an aspect is provided an isolated nucleic acid encoding an provided herein including embodiments thereof.

In embodiments, the antibody includes a light chain variable domain sequence and a heavy chain variable domain sequence, wherein the light chain variable domain sequence includes: a CDR L1, a CDR L2, and a CDR L3 as set forth in Table 1; and wherein the heavy chain variable domain sequence includes: a CDR H1, a CDR H2, and a CDR H3 as set forth in Table 1.

In embodiments, the light chain variable domain sequence is encoded by a nucleic acid sequence with at least 80%, 85%, 90%, 91%, 92%, 93%, 94%, 95%, 96%, 97%, 98%, 99%, or 100% sequence identity with a light chain variable nucleotide sequence as set forth in Table 1. In embodiments, the light chain variable domain sequence is encoded by a nucleic acid sequence with at least 80% sequence identity with a light chain variable nucleotide sequence as set forth in Table 1. In embodiments, the light chain variable domain sequence is encoded by a nucleic acid sequence with at least 85% sequence identity with a light chain variable nucleotide sequence as set forth in Table 1. In embodiments, the light chain variable domain sequence is encoded by a nucleic acid sequence with at least 90% sequence identity with a light chain variable nucleotide sequence as set forth in Table 1. In embodiments, the light chain variable domain sequence is encoded by a nucleic acid sequence with at least 91% sequence identity with a light chain variable nucleotide sequence as set forth in Table 1. In embodiments, the light chain variable domain sequence is encoded by a nucleic acid sequence with at least 92% sequence identity with a light chain variable nucleotide sequence as set forth in Table 1. In embodiments, the light chain variable domain sequence is encoded by a nucleic acid sequence with at least 93% sequence identity with a light chain variable nucleotide sequence as set forth in Table 1. In embodiments, the light chain variable domain sequence is encoded by a nucleic acid sequence with at least 94% sequence identity with a light chain variable nucleotide sequence as set forth in Table 1. In embodiments, the light chain variable domain sequence is encoded by a nucleic acid sequence with at least 95% sequence identity with a light chain variable nucleotide sequence as set forth in Table 1. In embodiments, the light chain variable domain sequence is encoded by a nucleic acid sequence with at least 96% sequence identity with a light chain variable nucleotide sequence as set forth in Table 1. In embodiments, the light chain variable domain sequence is encoded by a nucleic acid sequence with at least 97% sequence identity with a light chain variable nucleotide sequence as set forth in Table 1. In embodiments, the light chain variable domain sequence is encoded by a nucleic acid sequence with at least 98% sequence identity with a light chain variable nucleotide sequence as set forth in Table 1. In embodiments, the light chain variable domain sequence is encoded by a nucleic acid sequence with at least 99% sequence identity with a light chain variable nucleotide sequence as set forth in Table 1. In embodiments, the light chain variable domain sequence is encoded by a nucleic acid sequence with at least 100% sequence identity with a light chain variable nucleotide sequence as set forth in Table 1.

In embodiments, the heavy chain variable domain is encoded by a nucleic acid sequence with at least 80%, 85%, 90%, 91% 92%, 93%, 94%, 95%, 96%, 97%, 98%, 99%, or 100% sequence identity with a heavy chain variable nucleotide sequence as set forth in Table 1. In embodiments, the heavy chain variable domain is encoded by a nucleic acid sequence with at least 80% sequence identity with a heavy chain variable nucleotide sequence as set forth in Table 1. In embodiments, the heavy chain variable domain is encoded by a nucleic acid sequence with at least 85% sequence identity with a heavy chain variable nucleotide sequence as set forth in Table 1. In embodiments, the heavy chain variable domain is encoded by a nucleic acid sequence with at least 90% sequence identity with a heavy chain variable nucleotide sequence as set forth in Table 1. In embodiments, the light chain variable domain sequence is encoded by a nucleic acid sequence with at least 91% sequence identity with a light chain variable nucleotide sequence as set forth in Table 1. In embodiments, the light chain variable domain sequence is encoded by a nucleic acid sequence with at least 92% sequence identity with a light chain variable nucleotide sequence as set forth in Table 1. In embodiments, the light chain variable domain sequence is encoded by a nucleic acid sequence with at least 93% sequence identity with a light chain variable nucleotide sequence as set forth in Table 1. In embodiments, the light chain variable domain sequence is encoded by a nucleic acid sequence with at least 94% sequence identity with a light chain variable nucleotide sequence as set forth in Table 1. In embodiments, the heavy chain variable domain is encoded by a nucleic acid sequence with at 95% sequence identity with a heavy chain variable nucleotide sequence as set forth in Table 1. In embodiments, the light chain variable domain sequence is encoded by a nucleic acid sequence with at least 96% sequence identity with a light chain variable nucleotide sequence as set forth in Table 1. In embodiments, the light chain variable domain sequence is encoded by a nucleic acid sequence with at least 97% sequence identity with a light chain variable nucleotide sequence as set forth in Table 1. In embodiments, the heavy chain variable domain is encoded by a nucleic acid sequence with at least 98% sequence identity with a heavy chain variable nucleotide sequence as set forth in Table 1. In embodiments, the heavy chain variable domain is encoded by a nucleic acid sequence with at least 99% sequence identity with a heavy chain variable nucleotide sequence as set forth in Table 1. In embodiments, the heavy chain variable domain is encoded by a nucleic acid sequence with at least 100% sequence identity with a heavy chain variable nucleotide sequence as set forth in Table 1.

### Cell Compositions

The compositions provided herein including embodiments thereof include cellular compositions. The cells include nucleic acids provided herein including embodiments thereof as described in detail throughout this application (including the description above and in the examples section). Thus, in an aspect is provided a cell including the isolated nucleic acid provided herein including embodiments thereof. In embodiments, the isolated nucleic acid encodes an antibody provided herein including embodiments thereof. IN embodiments, the cell can be a lymphoid cell, a myeloid cell, or a stem cell. In embodiments, the (e.g., lymphoid) cell is a T cell or a B cell. In embodiments, the cell is a T cell. In embodiments, the cell is a B cell.

### Pharmaceutical Compositions

The compositions provided herein including embodiments thereof include pharmaceutical compositions. The pharmaceutical compositions include anti-CDH17 antibodies provided herein including embodiments thereof as described in detail throughout this application (including the description above and in the examples section). Thus, in an aspect is provided a pharmaceutical composition including a therapeutically effective amount of an antibody provided herein including embodiments thereof or a therapeutically effective amount of the isolated nucleic acid provided herein including embodiments thereof, and a pharmaceutically acceptable excipient.

In embodiments, the pharmaceutical composition is formulated in an aqueous solution at a concentration of 0.2 mg/mL to 2 mg/mL, 0.5 mg/mL to 1.5 mg/mL, 0.75 mg/mL to 1 mg/mL, or 1 mg/mL to 1.25 mg/mL. In embodiments, the pharmaceutical composition is formulated in an aqueous solution at a concentration of 0.2 mg/mL to 2 mg/mL. In embodiments, the pharmaceutical composition is formulated in an aqueous solution at a concentration of 0.5 mg/mL to 1.5 mg/mL. In embodiments, the pharmaceutical composition is formulated in an aqueous solution at a concentration of 0.75 mg/mL to 1 mg/mL. In embodiments, the pharmaceutical composition is formulated in an aqueous solution at a concentration of 1 mg/mL to 1.25 mg/mL. In embodiments, the pharmaceutical composition is formulated in an aqueous solution at a concentration of from 1.25 mg/mL to 200 mg/mL, from 2.50 mg/mL to 5.00 mg/mL, from 5.00 mg/mL to 10.0 mg/mL, from 10.0 mg/mL to 25.0 mg/mL, from 25.0 mg/mL to 50 mg/mL, from 50.0 mg/mL to 100 mg/mL, from 100 mg/mL to 150 mg/mL, or from 150 mg/mL to 200 mg/mL.

In embodiments, the pharmaceutical composition is formulated in an aqueous solution at a concentration of 0.2 mg/mL to 2 mg/mL. In embodiments, the pharmaceutical composition is formulated in an aqueous solution at a concentration of 0.3 mg/mL to 2 mg/mL. In embodiments, the pharmaceutical composition is formulated in an aqueous solution at a concentration of 0.4 mg/mL to 2 mg/mL. In embodiments, the pharmaceutical composition is formulated in an aqueous solution at a concentration of 0.5 mg/mL to 2 mg/mL. In embodiments, the pharmaceutical composition is formulated in an aqueous solution at a concentration of 0.6 mg/mL to 2 mg/mL. In embodiments, the pharmaceutical composition is formulated in an aqueous solution at a concentration of 0.7 mg/mL to 2 mg/mL. In embodiments, the pharmaceutical composition is formulated in an aqueous solution at a concentration of 0.8 mg/mL to 2 mg/mL. In embodiments, the pharmaceutical composition is formulated in an aqueous solution at a concentration of 0.9 mg/mL to 2 mg/mL. In embodiments, the pharmaceutical composition is formulated in an aqueous solution at a concentration of 1.0 mg/mL to 2 mg/mL. In embodiments, the pharmaceutical composition is formulated in an aqueous solution at a concentration of 1.1 mg/mL to 2 mg/mL. In embodiments, the pharmaceutical composition is formulated in an aqueous solution at a concentration of 1.2 mg/mL to 2 mg/mL. In embodiments, the pharmaceutical composition is formulated in an aqueous solution at a concentration of 1.3 mg/mL to 2 mg/mL. In embodiments, the pharmaceutical composition is formulated in an aqueous solution at a concentration of 1.4 mg/mL to 2 mg/mL. In embodiments, the pharmaceutical composition is formulated in an aqueous solution at a concentration of 1.5 mg/mL to 2 mg/mL. In embodiments, the pharmaceutical composition is formulated in an aqueous solution at a concentration of 1.6 mg/mL to 2 mg/mL. In embodiments, the pharmaceutical composition is formulated in an aqueous solution at a concentration of 1.7 mg/mL to 2 mg/mL. In embodiments, the pharmaceutical composition is formulated in an aqueous solution at a concentration of 1.8 mg/mL to 2 mg/mL. In embodiments, the pharmaceutical composition is formulated in an aqueous solution at a concentration of 1.9 mg/mL to 2 mg/mL.

In embodiments, the pharmaceutical composition is formulated in an aqueous solution at a concentration of 0.2 mg/mL to 200 mg/mL. In embodiments, the pharmaceutical composition is formulated in an aqueous solution at a concentration of 0.2 mg/mL to 1.8 mg/mL. In embodiments, the pharmaceutical composition is formulated in an aqueous solution at a concentration of 0.2 mg/mL to 1.7 mg/mL. In embodiments, the pharmaceutical composition is formulated in an aqueous solution at a concentration of 0.2 mg/mL to 1.6 mg/mL. In embodiments, the pharmaceutical composition is formulated in an aqueous solution at a concentration of 0.2 mg/mL to 1.5 mg/mL. In embodiments, the pharmaceutical composition is formulated in an aqueous solution at a concentration of 0.2 mg/mL to 1.4 mg/mL. In embodiments, the pharmaceutical composition is formulated in an aqueous solution at a concentration of 0.2 mg/mL to 1.3 mg/mL. In embodiments, the pharmaceutical composition is formulated in an aqueous solution at a concentration of 0.2 mg/mL to 1.2 mg/mL. In embodiments, the pharmaceutical composition is formulated in an aqueous solution at a concentration of 0.2 mg/mL to 1.1 mg/mL. In embodiments, the pharmaceutical composition is formulated in an aqueous solution at a concentration of 0.2 mg/mL to 1.0 mg/mL. In embodiments, the pharmaceutical composition is formulated in an aqueous solution at a concentration of 0.2 mg/mL to 0.9 mg/mL. In embodiments, the pharmaceutical composition is formulated in an aqueous solution at a concentration of 0.2 mg/mL to 0.8 mg/mL. In embodiments, the pharmaceutical composition is formulated in an aqueous solution at a concentration of 0.2 mg/mL to 0.7 mg/mL. In embodiments, the pharmaceutical composition is formulated in an aqueous solution at a concentration of 0.2 mg/mL to 0.6 mg/mL. In embodiments, the pharmaceutical composition is formulated in an aqueous solution at a concentration of 0.2 mg/mL to 0.5 mg/mL. In embodiments, the pharmaceutical composition is formulated in an aqueous solution at a concentration of 0.2 mg/mL to 0.4 mg/mL. In embodiments, the pharmaceutical composition is formulated in an aqueous solution at a concentration of 0.2 mg/mL to 0.3 mg/mL.

In embodiments, the pharmaceutical composition is formulated in an aqueous solution at a concentration of 0.5 mg/mL to 1.5 mg/mL. In embodiments, the pharmaceutical composition is formulated in an aqueous solution at a concentration of 0.6 mg/mL to 1.5 mg/mL. In embodiments, the pharmaceutical composition is formulated in an aqueous solution at a concentration of 0.7 mg/mL to 1.5 mg/mL. In embodiments, the pharmaceutical composition is formulated in an aqueous solution at a concentration of 0.8 mg/mL to 1.5 mg/mL. In embodiments, the pharmaceutical composition is formulated in an aqueous solution at a concentration of 0.9 mg/mL to 1.5 mg/mL. In embodiments, the pharmaceutical composition is formulated in an aqueous solution at a concentration of 1.0 mg/mL to 1.5 mg/mL. In embodiments, the pharmaceutical composition is formulated in an aqueous solution at a concentration of 1.1 mg/mL to 1.5 mg/mL. In embodiments, the pharmaceutical composition is formulated in an aqueous solution at a concentration of 1.2 mg/mL to 1.5 mg/mL. In embodiments, the pharmaceutical composition is formulated in an aqueous solution at a concentration of 1.3 mg/mL to 1.5 mg/mL. In embodiments, the pharmaceutical composition is formulated in an aqueous solution at a concentration of 1.4 mg/mL to 1.5 mg/mL.

In embodiments, the pharmaceutical composition is formulated in an aqueous solution at a concentration of 0.5 mg/mL to 1.4 mg/mL. In embodiments, the pharmaceutical composition is formulated in an aqueous solution at a concentration of 0.5 mg/mL to 1.3 mg/mL. In embodiments, the pharmaceutical composition is formulated in an aqueous solution at a concentration of 0.5 mg/mL to 1.2 mg/mL. In embodiments, the pharmaceutical composition is formulated in an aqueous solution at a concentration of 0.5 mg/mL to 1.1 mg/mL. In embodiments, the pharmaceutical composition is formulated in an aqueous solution at a concentration of 0.5 mg/mL to 1.0 mg/mL. In embodiments, the pharmaceutical composition is formulated in an aqueous solution at a concentration of 0.5 mg/mL to 0.9 mg/mL. In embodiments, the pharmaceutical composition is formulated in an aqueous solution at a concentration of 0.5 mg/mL to 0.8 mg/mL. In embodiments, the pharmaceutical composition is formulated in an aqueous solution at a concentration of 0.5 mg/mL to 0.7 mg/mL. In embodiments, the pharmaceutical composition is formulated in an aqueous solution at a concentration of 0.5 mg/mL to 0.6 mg/mL.

In embodiments, the pharmaceutical composition is formulated in an aqueous solution at a concentration of 0.75 mg/mL to 1 mg/mL. In embodiments, the pharmaceutical composition is formulated in an aqueous solution at a concentration of 0.80 mg/mL to 1 mg/mL. In embodiments, the pharmaceutical composition is formulated in an aqueous solution at a concentration of 0.85 mg/mL to 1 mg/mL. In embodiments, the pharmaceutical composition is formulated in an aqueous solution at a concentration of 0.90 mg/mL to 1 mg/mL. In embodiments, the pharmaceutical composition is formulated in an aqueous solution at a concentration of 0.95 mg/mL to 1 mg/mL.

In embodiments, the pharmaceutical composition is formulated in an aqueous solution at a concentration of 0.75 mg/mL to 0.95 mg/mL. In embodiments, the pharmaceutical composition is formulated in an aqueous solution at a concentration of 0.75 mg/mL to 0.90 mg/mL. In embodiments, the pharmaceutical composition is formulated in an aqueous solution at a concentration of 0.75 mg/mL to 0.85 mg/mL. In embodiments, the pharmaceutical composition is formulated in an aqueous solution at a concentration of 0.75 mg/mL to 0.80 mg/mL.

In embodiments, the pharmaceutical composition is formulated in an aqueous solution at a concentration of 1 mg/mL to 1.25 mg/mL. In embodiments, the pharmaceutical composition is formulated in an aqueous solution at a concentration of 1.05 mg/mL to 1.25 mg/mL. In embodiments, the pharmaceutical composition is formulated in an aqueous solution at a concentration of 1.10 mg/mL to 1.25 mg/mL. In embodiments, the pharmaceutical composition is formulated in an aqueous solution at a concentration of 1.15 mg/mL to 1.25 mg/mL. In embodiments, the pharmaceutical composition is formulated in an aqueous solution at a concentration of 1.20 mg/mL to 1.25 mg/mL.

In embodiments, the pharmaceutical composition is formulated in an aqueous solution at a concentration of 1 mg/mL to 1.20 mg/mL. In embodiments, the pharmaceutical composition is formulated in an aqueous solution at a concentration of 1 mg/mL to 1.15 mg/mL. In embodiments, the pharmaceutical composition is formulated in an aqueous solution at a concentration of 1 mg/mL to 1.10 mg/mL. In embodiments, the pharmaceutical composition is formulated in an aqueous solution at a concentration of 1 mg/mL to 1.05 mg/mL.

In embodiments, the pharmaceutical composition is formulated for a delivery method selected from intratumoral delivery, intramuscular delivery, intravascular delivery, peritoneal delivery, or subcutaneous delivery.

In embodiments, the pharmaceutical composition further includes a therapeutically effective amount of an antineoplastic agent.

### Methods and Uses

The compositions (e.g., anti-CDH17 antibodies and recombinant proteins) provided herein, including embodiments thereof, are contemplated as providing effective treatment for diseases such as cancer. Thus, in an aspect is provided a method of treating a subject in need thereof, the method including administering to a subject a therapeutically effective amount of the antibody provided herein including embodiments thereof. In embodiments, the methods disclosed herein further include administering a therapeutically effective amount of an antineoplastic agent.

In embodiments, the effective amount of the antibody and the effective amount of the antineoplastic agent are a combined synergistic amount.

In embodiments, the subject has a tumor or a cancer, is at risk of developing the tumor or the cancer, or is suspected of having the tumor or the cancer. In embodiments, the subject has a tumor or a cancer. In embodiments, the subject has a tumor. In embodiments, the subject has a cancer. In embodiments, the subject is at risk of developing the tumor or the cancer. In embodiments, the subject is at risk of developing the tumor. In embodiments, the subject is at risk of developing the cancer. In embodiments, the subject is suspected of having the tumor or the cancer. In embodiments, the subject is suspected of having the tumor. In embodiments, the subject is suspected of having the cancer.

In embodiments, the cancer is a gastrointestinal cancer, a colorectal cancer, a stomach cancer, a pancreas cancer, a neuroendocrine cancer, or an ovarian cancer. In embodiments, the cancer is a gastrointestinal cancer. In embodiments, the cancer is a colorectal cancer. In embodiments, the cancer is a stomach cancer. In embodiments, the cancer is a pancreas cancer. In embodiments, the cancer is a neuroendocrine cancer. In embodiments, the cancer is an ovarian cancer.

In embodiments, the subject has been identified as having a tumor or cancer, or has been identified as at risk of develop the tumor or the cancer. In embodiments, the subject has been identified as having a tumor or cancer. In embodiments, the subject has been identified as having a tumor. In embodiments, the subject has been identified as having a cancer. In embodiments, the subject has been identified as at risk of developing a tumor or a cancer. In embodiments, the subject has been identified as at risk of developing a tumor. In embodiments, the subject has been identified as at risk of developing a cancer.

In embodiments, the antibody is administered to the subject at a concentration of 0.2 mg/mL to 2 mg/mL, 0.5 mg/mL to 1.5 mg/mL, 0.75 mg/mL to 1 mg/mL, or 1 mg/mL to 1.25 mg/mL. In embodiments, the antibody is administered to the subject at a concentration of 0.2 mg/mL to 2 mg/mL. In embodiments, the antibody is administered to the subject at a concentration of 0.5 mg/mL to 1.5 mg/mL. In embodiments, the antibody is administered to the subject at a concentration of 0.75 mg/mL to 1 mg/mL. In embodiments, the antibody is administered to the subject at a concentration of 1 mg/mL to 1.25 mg/mL.

In embodiments, the antibody is administered to the subject at a concentration of 0.2 mg/mL to 2 mg/mL. In embodiments, the antibody is administered to the subject at a concentration of 0.3 mg/mL to 2 mg/mL. In embodiments, the antibody is administered to the subject at a concentration of 0.4 mg/mL to 2 mg/mL. In embodiments, the antibody is administered to the subject at a concentration of 0.5 mg/mL to 2 mg/mL. In embodiments, the antibody is administered to the subject at a concentration of 0.6 mg/mL to 2 mg/mL. In embodiments, the antibody is administered to the subject at a concentration of 0.7 mg/mL to 2 mg/mL. In embodiments, the antibody is administered to the subject at a concentration of 0.8 mg/mL to 2 mg/mL. In embodiments, the antibody is administered to the subject at a concentration of 0.9 mg/mL to 2 mg/mL. In embodiments, the antibody is administered to the subject at a concentration of 1.0 mg/mL to 2 mg/mL. In embodiments, the antibody is administered to the subject at a concentration of 1.1 mg/mL to 2 mg/mL. In embodiments, the antibody is administered to the subject at a concentration of 1.2 mg/mL to 2 mg/mL. In embodiments, the antibody is administered to the subject at a concentration of 1.3 mg/mL to 2 mg/mL. In embodiments, the antibody is administered to the subject at a concentration of 1.4 mg/mL to 2 mg/mL. In embodiments, the antibody is administered to the subject at a concentration of 1.5 mg/mL to 2 mg/mL. In embodiments, the antibody is administered to the subject at a concentration of 1.6 mg/mL to 2 mg/mL. In embodiments, the antibody is administered to the subject at a concentration of 1.7 mg/mL to 2 mg/mL. In embodiments, the antibody is administered to the subject at a concentration of 1.8 mg/mL to 2 mg/mL. In embodiments, the antibody is administered to the subject at a concentration of 1.9 mg/mL to 2 mg/mL.

In embodiments, the antibody is administered to the subject at a concentration of 0.2 mg/mL to 1.9 mg/mL. In embodiments, the antibody is administered to the subject at a concentration of 0.2 mg/mL to 1.8 mg/mL. In embodiments, the antibody is administered to the subject at a concentration of 0.2 mg/mL to 1.7 mg/mL. In embodiments, the antibody is administered to the subject at a concentration of 0.2 mg/mL to 1.6 mg/mL. In embodiments, the antibody is administered to the subject at a concentration of 0.2 mg/mL to 1.5 mg/mL. In embodiments, the antibody is administered to the subject at a concentration of 0.2 mg/mL to 1.4 mg/mL. In embodiments, the antibody is administered to the subject at a concentration of 0.2 mg/mL to 1.3 mg/mL. In embodiments, the antibody is administered to the subject at a concentration of 0.2 mg/mL to 1.2 mg/mL. In embodiments, the antibody is administered to the subject at a concentration of 0.2 mg/mL to 1.1 mg/mL. In embodiments, the antibody is administered to the subject at a concentration of 0.2 mg/mL to 1.0 mg/mL. In embodiments, the antibody is administered to the subject at a concentration of 0.2 mg/mL to 0.9 mg/mL. In embodiments, the antibody is administered to the subject at a concentration of 0.2 mg/mL to 0.8 mg/mL. In embodiments, the antibody is administered to the subject at a concentration of 0.2 mg/mL to 0.7 mg/mL. In embodiments, the antibody is administered to the subject at a concentration of 0.2 mg/mL to 0.6 mg/mL. In embodiments, the antibody is administered to the subject at a concentration of 0.2 mg/mL to 0.5 mg/mL. In embodiments, the antibody is administered to the subject at a concentration of 0.2 mg/mL to 0.4 mg/mL. In embodiments, the antibody is administered to the subject at a concentration of 0.2 mg/mL to 0.3 mg/mL.

In embodiments, the antibody is administered to the subject at a concentration of 0.5 mg/mL to 1.5 mg/mL. In embodiments, the antibody is administered to the subject at a concentration of 0.6 mg/mL to 1.5 mg/mL. In embodiments, the antibody is administered to the subject at a concentration of 0.7 mg/mL to 1.5 mg/mL. In embodiments, the antibody is administered to the subject at a concentration of 0.8 mg/mL to 1.5 mg/mL. In embodiments, the antibody is administered to the subject at a concentration of 0.9 mg/mL to 1.5 mg/mL. In embodiments, the antibody is administered to the subject at a concentration of 1.0 mg/mL to 1.5 mg/mL. In embodiments, the antibody is administered to the subject at a concentration of 1.1 mg/mL to 1.5 mg/mL. In embodiments, the antibody is administered to the subject at a concentration of 1.2 mg/mL to 1.5 mg/mL. In embodiments, the antibody is administered to the subject at a concentration of 1.3 mg/mL to 1.5 mg/mL. In embodiments, the antibody is administered to the subject at a concentration of 1.4 mg/mL to 1.5 mg/mL.

In embodiments, the antibody is administered to the subject at a concentration of 0.5 mg/mL to 1.4 mg/mL. In embodiments, the antibody is administered to the subject at a concentration of 0.5 mg/mL to 1.3 mg/mL. In embodiments, the antibody is administered to the subject at a concentration of 0.5 mg/mL to 1.2 mg/mL. In embodiments, the antibody is administered to the subject at a concentration of 0.5 mg/mL to 1.1 mg/mL. In embodiments, the antibody is administered to the subject at a concentration of 0.5 mg/mL to 1.0 mg/mL. In embodiments, the antibody is administered to the subject at a concentration of 0.5 mg/mL to 0.9 mg/mL. In embodiments, the antibody is administered to the subject at a concentration of 0.5 mg/mL to 0.8 mg/mL. In embodiments, the antibody is administered to the subject at a concentration of 0.5 mg/mL to 0.7 mg/mL. In embodiments, the antibody is administered to the subject at a concentration of 0.5 mg/mL to 0.6 mg/mL.

In embodiments, the antibody is administered to the subject at a concentration of 0.75 mg/mL to 1 mg/mL. In embodiments, the antibody is administered to the subject at a concentration of 0.80 mg/mL to 1 mg/mL. In embodiments, the antibody is administered to the subject at a concentration of 0.85 mg/mL to 1 mg/mL. In embodiments, the antibody is administered to the subject at a concentration of 0.90 mg/mL to 1 mg/mL. In embodiments, the antibody is administered to the subject at a concentration of 0.95 mg/mL to 1 mg/mL.

In embodiments, the antibody is administered to the subject at a concentration of 0.75 mg/mL to 0.95 mg/mL. In embodiments, the antibody is administered to the subject at a concentration of 0.75 mg/mL to 0.90 mg/mL. In embodiments, the antibody is administered to the subject at a concentration of 0.75 mg/mL to 0.85 mg/mL. In embodiments, the antibody is administered to the subject at a concentration of 0.75 mg/mL to 0.80 mg/mL.

In embodiments, the antibody is administered to the subject at a concentration of 1 mg/mL to 1.25 mg/mL. In embodiments, the antibody is administered to the subject at a concentration of 1.05 mg/mL to 1.25 mg/mL. In embodiments, the antibody is administered to the subject at a concentration of 1.10 mg/mL to 1.25 mg/mL. In embodiments, the antibody is administered to the subject at a concentration of 1.15 mg/mL to 1.25 mg/mL. In embodiments, the antibody is administered to the subject at a concentration of 1.20 mg/mL to 1.25 mg/mL.

In embodiments, the antibody is administered to the subject at a concentration of 1 mg/mL to 1.20 mg/mL. In embodiments, the antibody is administered to the subject at a concentration of 1 mg/mL to 1.15 mg/mL. In embodiments, the antibody is administered to the subject at a concentration of 1 mg/mL to 1.10 mg/mL. In embodiments, the antibody is administered to the subject at a concentration of 1 mg/mL to 1.05 mg/mL.

In another aspect is the use of the antibody provided herein including embodiments thereof in the manufacture of a medicament for the treatment of a tumor or cancer.

In embodiments, the anti-CDH17 antibody provided herein including embodiments thereof targets tumor cells or cancer cells through antibody-dependent cell cytotoxicity (ADCC). In embodiments, the anti-CDH17 antibody provided herein including embodiments thereof engages immune cells including natural killer (NK) cells through Fc interactions to enhance targeting of tumor cells or cancer cells which over-express CDH17 through antibody-dependent cell cytotoxicity (ADCC).

In embodiments, the anti-CDH17 antibody provided herein including embodiments thereof targets tumor cells or cancer cells through complement-dependent cytotoxicity (CDC). In embodiments, the anti-CDH17 antibody provided herein including embodiments thereof recruits complement proteins and activates the complement cascade through Fc interactions and enhance targeting of tumor cells or cancer cells which over-express CDH17 through complement-dependent cytotoxicity (CDC).

In embodiments, the anti-CDH17 antibody provided herein including embodiments thereof targets tumor cells or cancer cells through antibody-dependent cell phagocytosis (ADCP). In embodiments, the anti-CDH17 antibody provided herein including embodiments thereof engages macrophages and neutrophils through Fc interactions to enhance targeting of tumor cells or cancer cells which over-express CDH17 through antibody-dependent cell phagocytosis (ADCP).

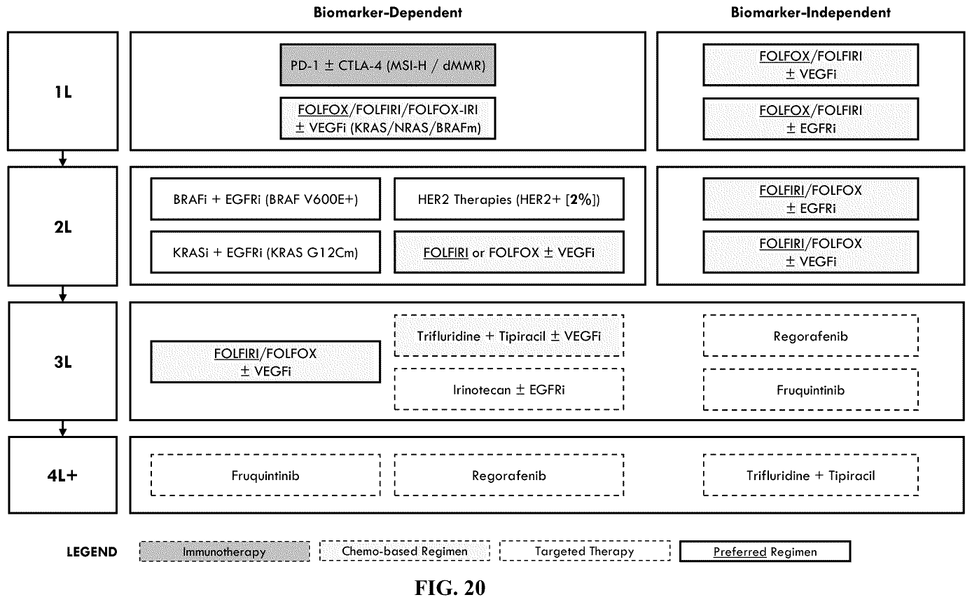

Compositions described herein (e.g., one or more of the AT-20 family antibodies or AT-20 family antibody-drug conjugates described herein) can be used to treat a subject in combination with one or more additional compositions described herein and/or one or more anti-tumor or anti-cancer drugs or therapies, including chemotherapies. In embodiments, a method of treating a subject in need thereof (e.g., a subject having or suspected of having a cancer or tumor, including gastrointestinal cancers or tumors such as colorectal cancers or tumors, pancreatic cancers or tumors, esophageal cancers or tumors, or gastric cancers or tumors) can comprise administering an AT-20 family antibody and/or an AT-20 family antibody drug conjugate to the subject and also administering a chemotherapy treatment or regimen to the subject. In some cases, the chemotherapy treatment or regimen can be a first line (1L) chemotherapy treatment or regimen. In some cases, the chemotherapy treatment or regimen can be a second line (2L) chemotherapy treatment or regimen. In some cases, the chemotherapy treatment or regimen can be a third line (3L) chemotherapy treatment or regimen. In some cases, the chemotherapy treatment or regimen can be a fourth line or later (4L+) chemotherapy treatment or regimen. In some cases, a method of treating a subject having or suspected of having a cancer or tumor can be selected based on whether the cancer or tumor is biomarker dependent or biomarker independent. In embodiments, an AT-20 family antibody or AT-20 family antibody drug conjugate described herein can be administered to subjects with microsatellite instability-high (MSI-H) presentation and/or deficient mismatch repair (dMMR) presentation (e.g., in biomarker-dependent tumors and cancers). In embodiments, an AT-20 antibody (e.g., a naked AT-20 antibody) or an AT-20 antibody ADC described herein can be administered to a subject with FOLFOX, FOLFIRI, or FOLFOX-IRI chemotherapy regimens, e.g., along with vascular endothelial growth factor inhibitors (VEGFi). In some cases, such treatments can be used to treat subjects with biomarker independent conditions, for example, along with AT-20 antibody cotreatment (e.g., as an AT-20 antibody ADC described herein or as a naked AT-20 antibody). A method of treating a subject having or suspected of having a cancer or tumor can include treatment with trifluridine, tipiracil and VEGFi, or with Irinotecan with epidermal growth factor receptor inhibitors (EGFRi), or with regorafenib, or with fruquinitinib. As contemplated herein, AT-20 antibodies and/or AT-20 antibody drug conjugates described herein can be used in regimens for treating a subject having or at risk of having a cancer or tumor (e.g., a gastrointestinal cancer or tumor, including a colorectal cancer or tumor, a pancreatic cancer or tumor, an esophageal cancer or tumor, a gastric cancer or tumor) in place of one or more treatment options shown in FIG. 20 for the patient population indicated in in FIG. 20 (e.g., biomarker dependent cancers and tumors with MSI-H and/or dMMR or biomarker independent cancers and tumors) or in conjunction with a listed treatment (e.g., FOLFOX, FOLFIRI, FOLFOX-IRI, VEGFi, EGFRi, irinotecan, trifluiridine, tipiracil, fruquinitib, or regorafenib).

### EXAMPLES

### Example 1

Cadherin-17 (CDH17), also known as Liver-Intestine Cadherin (LI-Cadherin) is a member of the cadherin superfamily. It is a calcium-dependent, membrane-associated glycoprotein. It is a type 1 transmembrane protein containing 7 cadherin domains, a transmembrane region, and one cytoplasmic domain. Its main function is to mediate calcium-dependent cell adhesion.

According to TCGA, over-expression of CDH17 is commonly found in colorectal cancer, stomach cancer, and pancreatic cancer (GEPIA 2). High expression of CDH17 in GI tract cancers is also reflected in the high expression of CDH17 mRNA among cancer cell lines (CCLE, Depmap). The highest CDH17-expressing cell lines exclusively originate from GI tract tumors. Furthermore, high expression level of CDH17 protein has been confirmed by IHC staining as shown at The Human Protein Atlas (CDH17 protein expression summary—The Human Protein Atlas), suggesting that sufficient and effective targeting CDH17 by antibody-based therapy is possible.

In normal tissues, CDH17 is highly expressed in colon and small intestines (GTEx Portal). High level expression of a target in normal tissue can be a concern for ADC therapies. However, CDH17 expression is limited to the lateral side of the polarized epithelial cells, not on either apical nor basal side of the cells under normal conditions (Feng Z et al., doi.org/10.1038/s43018-022-00344-7). The lateral sides of the cells form cell-cell tight junctions and is not accessible to the antibody. In cancerous tissue, this polarity is lost and CDH17 expression is found to be on all sides of the cells, thus the susceptibility of tumor cells to anti-CDH17 ADC.

### Example 2: Immunization with Protein

This example shows immunization of mouse subjects with anti-CDH17 antibody compositions described herein.

Mouse subjects are subjected to a 1^{st} immunization at 1 site comprising intraperitoneal injection of 50 micrograms (g) of antigen (protein) with Freund's complete adjuvant, which comprises water in oil emulsion and inactivated and dried mycobacteria.

The following week, the subject is then subjected to a 2^{nd} immunization at 1 site comprising intraperitoneal injection of 50 micrograms (g) of antigen (protein) with Freund's incomplete adjuvant, which comprises water in oil emulsion.

The following week, the subject is then subjected to a 3^{rd} immunization at 1 site comprising intraperitoneal injection of 50 micrograms (g) of antigen (protein) with Freund's incomplete adjuvant.

The following week, the subject is then subjected to a 4^{th} immunization at 1 site comprising intraperitoneal injection of 50 micrograms (g) of antigen (protein) with Freund's incomplete adjuvant.

The subject is subsequently subjected to a final booster immunization injected intraperitoneally using 85 micrograms (g) of protein in phosphate buffered saline (PBS). After 72 hours the spleen is collected for analysis.

### Example 3: Flow Cytometry Cell Surface Affinity Binding

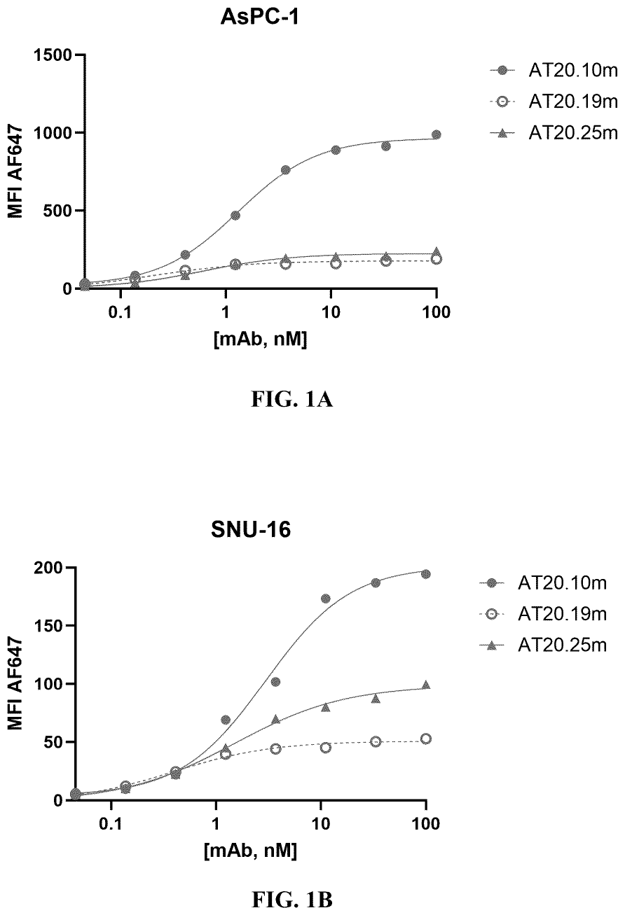

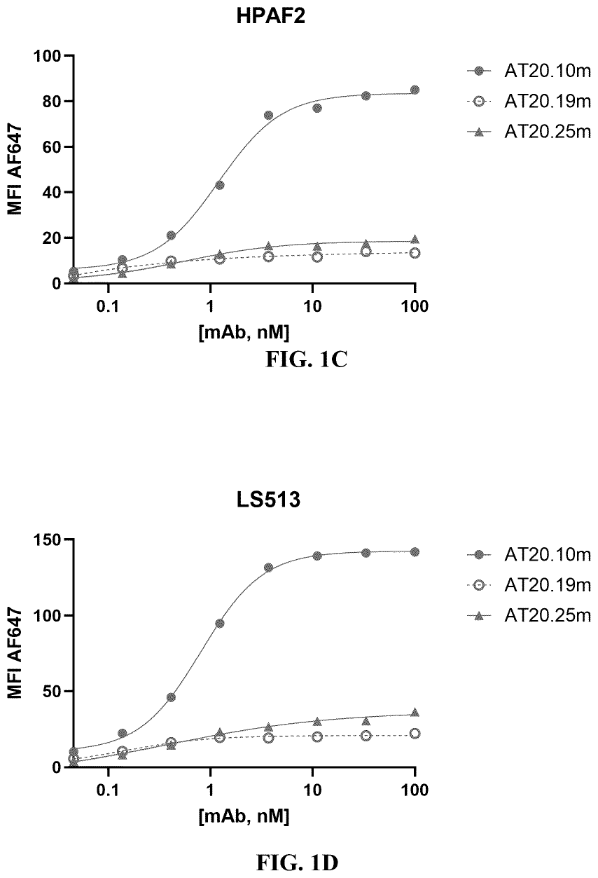

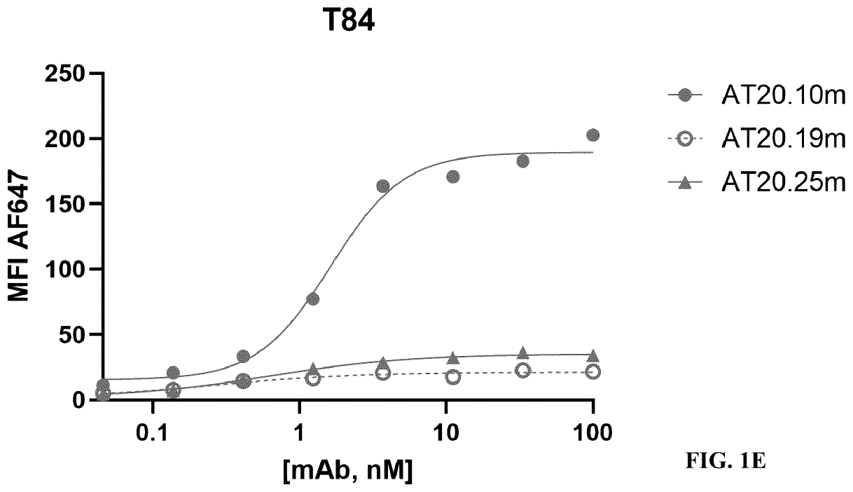

FIGS. 1A-IE, 2A-2D, 3A-3B, 4A-4D, and 5A-5B show in vitro AT-20 series asset binding to cell surface CDH17 expressed on AsPC-1 (pancreatic tumor cells), SNU-16 (gastric cancer cells), HPAF2 (pancreatic tumor cells), LS513 (colorectal tumor cells), T84 (colorectal tumor cells), and Expi293 cells transfected with human CDH17 and cynomolgus CDH17. Data are presented as mean fluorescence intensity (MFI) of AlexaFluor 647 (AF647) or AlexaFluor 488 (AF488) fluorophores on secondary antibodies bound to the AT-20 family primary antibodies detected during flow cytometry analysis after incubation of cells with AT-20 series antibodies at varying concentrations of antibody (shown in nanomolar (nM) concentration).

FIGS. 1A, 1B, 1C, 1D, and 1E show cell surface binding of murine AT-20 antibody clones (AT-20.10m (filled circles), AT-20.19m (open circles), and AT-20.25m (triangles)) to AsPC-1 pancreatic tumor cells, SNU-16 gastric cancer cells, HPAF2 pancreatic tumor cells, LS513 colorectal tumor cells, and T84 colorectal tumor cells, respectively. Positive binding to tumor and cancer cells was observed for all clones shown in these data. EC50 values for AT20.10m, AT20.19m, and AT20.25m binding to AsPC-1pancreatic tumor cells were calculated to be 1.3 nanomolar (nM), 0.2 nM, and 0.6 nM, respectively. EC50 values for AT20.10m, AT20.19m, and AT20.25m binding to SNU-16 gastric cancer cells were calculated to be 3.0 nM, 0.4 nM, and 1.5 nM, respectively. EC50 values for AT20.10m and AT20.25m binding to HPAF2 pancreatic tumor cells were calculated to be 1.2 nM and 0.5 nM, respectively. EC50 values for AT20.10m, AT20.19m, and AT20.25m binding to LS513 colorectal tumor cells were calculated to be 0.8 nM, 0.1 nM, and 0.4 nM, respectively. EC50 values for AT20.10m, AT20.19m, and AT20.25m binding to T84 colorectal tumor cells were calculated to be 1.6 nM, 0.3 nM, and 0.6 nM, respectively.

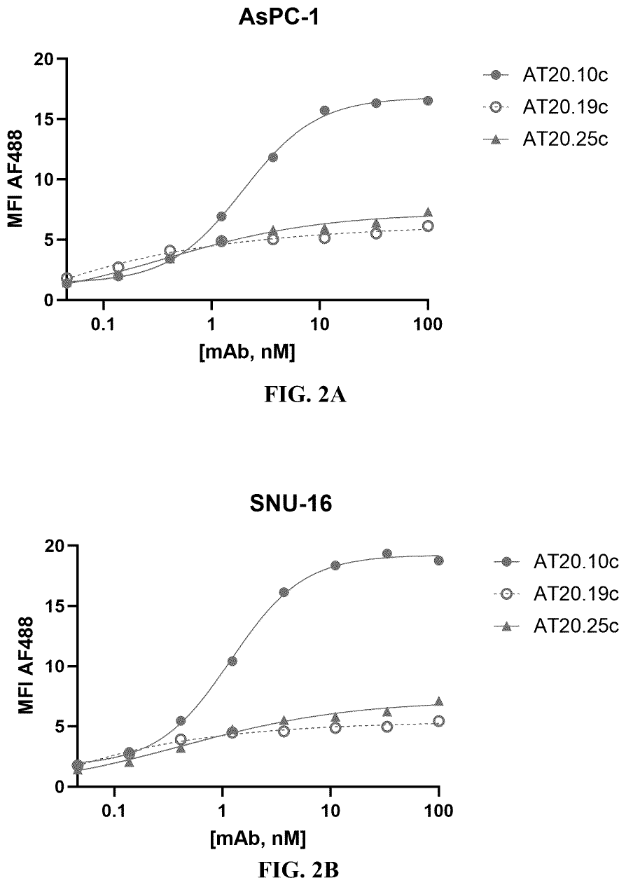

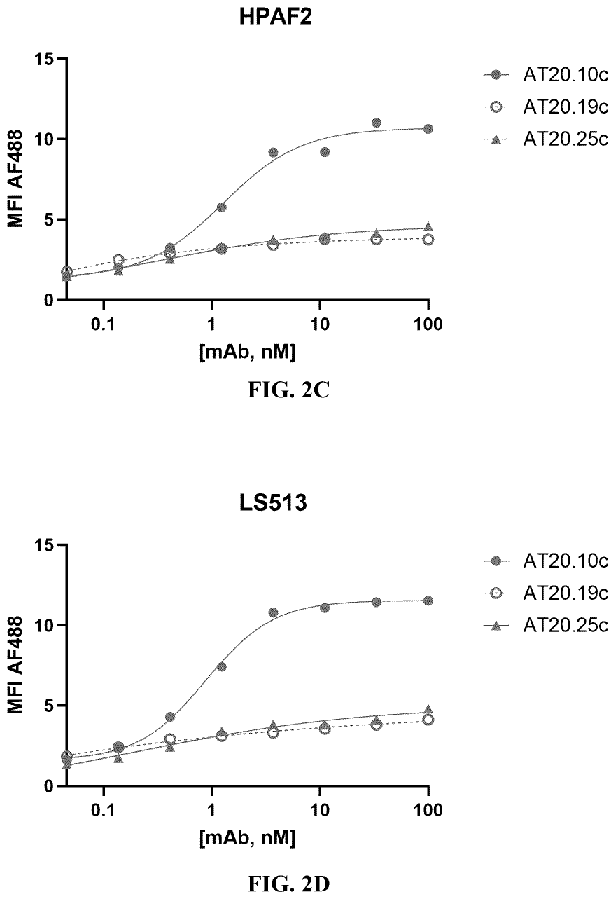

FIGS. 2A, 2B, 2C, and 2D show cell surface binding of chimeric AT-20 antibody clones (AT-20.10c (filled circles), AT-20.19c (open circles), and AT-20.25c (triangles)) to AsPC-1 pancreatic tumor cells, SNU-16 gastric cancer cells, HPAF2 pancreatic tumor cells, and LS513 colorectal tumor cells, respectively. EC50 values for AT20.10c and AT20.25c binding to AsPC-1 pancreatic tumor cells were calculated to be 1.9 nanomolar (nM) and 0.4 nM, respectively. EC50 values for AT20.10c and AT20.25c binding to SNU-16 gastric cancer cells were calculated to be 1.2 nM and 0.4 nM, respectively. EC50 values for AT20.10c and AT20.25c binding to HPAF2 pancreatic tumor cells were calculated to be 1.3 nM and 0.4 nM, respectively. EC50 values for AT20.10c and AT20.25c binding to LS513 colorectal tumor cells were found to be 0.9 nM and 0.2 nM, respectively. Dose-dependent binding of AT20.19c antibodies to AsPC-1 pancreatic tumor cells, SNU-16 gastric cancer cells, HPAF2 pancreatic tumor cells, and LS513 colorectal tumor cells were observed in the experimental data, but EC50 values could not be calculated from these data.

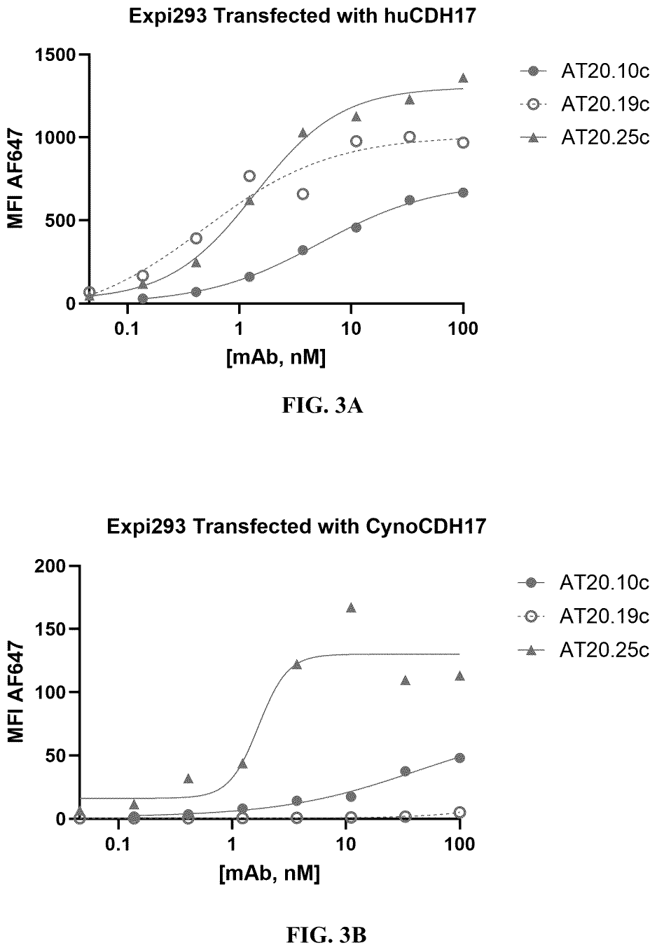

FIG. 3A shows cell surface binding of chimeric AT-20 antibody clones (AT-20.10c (filled circles), AT-20.19c (open circles), and AT-20.25c (triangles)) to Expi293 cells transfected with a human CDH17 expression plasmid so as to induce surface expression of human CDH17 protein. Positive binding was observed for all tested antibody clones but was most pronounced for AT-20.25c and AT-20.19c clone antibodies. EC50 values for AT20.10c, AT20.19c, and AT20.25c binding to cells transfected with human CDH17 expression plasmids were calculated to be 5.3 nanomolar (nM), 0.5 nM, and 1.4 nM, respectively. FIG. 3B shows cell surface binding of chimeric AT-20 antibody clones (AT-20.10c, AT-20.19c, and AT-20.25c) to Expi293 cells transfected with a cynomolgus CDH17 expression plasmid so as to induce surface expression of cynomolgus CDH17 protein. Positive binding was observed but was most pronounced for AT-20.25c clone antibody. EC50 values for AT20.10c and AT20.25c binding to cells transfected with cynomolgus CDH17 expression plasmid (“CynoCDH17”) were calculated to be 41.4 nM and 1.7 nM, respectively. No EC50 value was calculated from AT20.19c binding data.

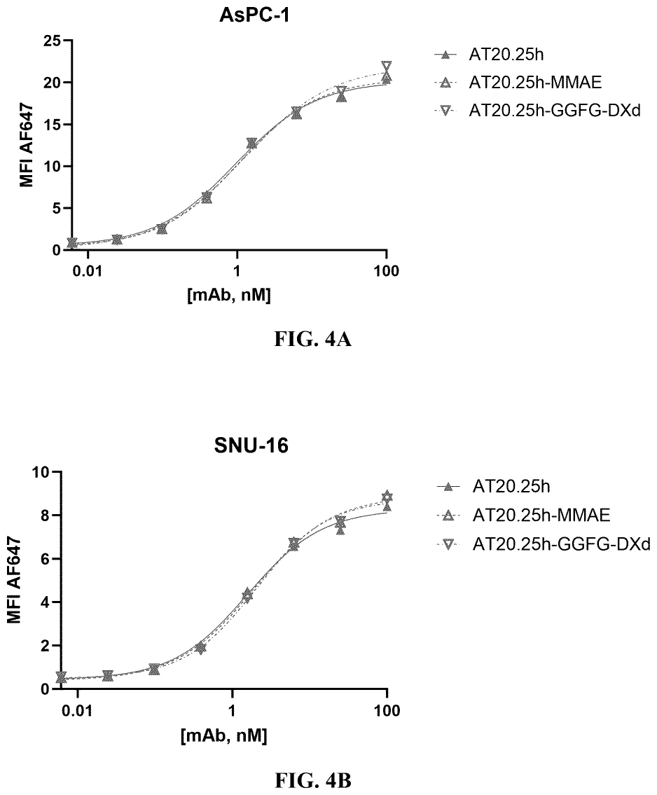

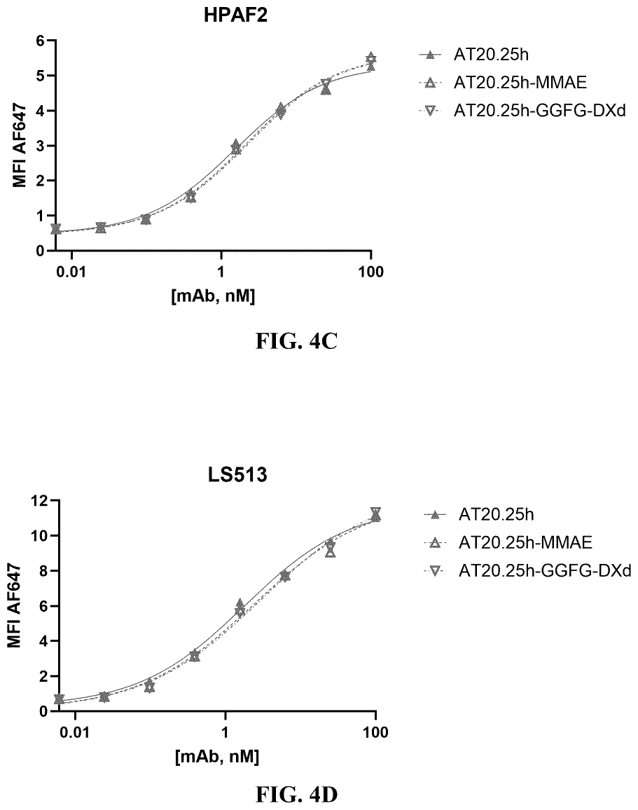

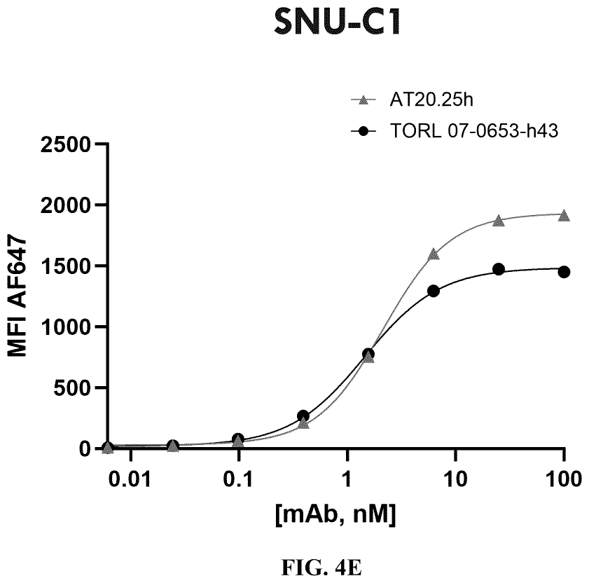

FIGS. 4A, 4B, 4C and 4D show high affinity cell surface binding of humanized AT-20.25 antibodies (naked AT-20.25h antibody (no payload) (filled triangles pointing up), AT-20.25h antibody conjugated to MMAE (empty triangles pointing up), and AT-20.25h conjugated to Deruxtecan (GGFG-DXd) (empty triangles pointing down)) to AsPC-1 pancreatic tumor cells, SNU-16 gastric tumor cells, HPAF2 pancreatic tumor cells, and LS513 colorectal tumor cells, respectively. Positive binding of AT20.25h (no payload), AT20.25h-MMAE, and AT20.25h-DXd antibodies to each of the tested cell types were observed. EC50 values for AT20.25h (no payload), AT20.25h-MMAE, and AT20.25h-GGFG-DXd binding to AsPC-1 pancreatic tumor cells were calculated to be 1.0 nanomolar (nM), 1.1 nM, and 1.3 nM, respectively. EC50 values for AT20.25h, AT20.25h-MMAE, and AT20.25h-GGFG-DXd binding to SNU-16 gastric tumor cells were calculated to be 1.6 nM, 1.9 nM, and 2.0 nM, respectively. EC50 values for AT20.25h (no payload), AT20.25h-MMAE, and AT20.25h-GGFG-DXd binding to HPAF2 pancreatic tumor cells were calculated to be 1.5 nM, 2.2 nM, and 2.3 nM, respectively. EC50 values for AT20.25h (no payload), AT20.25h-MMAE, and AT20.25h-GGFG-DXd binding to LS513 colorectal tumor cells were calculated to be 1.8 nM, 2.4 nM, and 2.9 nM, respectively. FIG. 4E shows binding of humanized AT-20.25 antibody (AT-20.25h) to SNU-C1 colorectal cancer cells in vitro (EC50, 2.1 nM), which was comparable to TORL 07-0653-h43 binding to SNU-C1 cells (EC50, 1.4 nM).

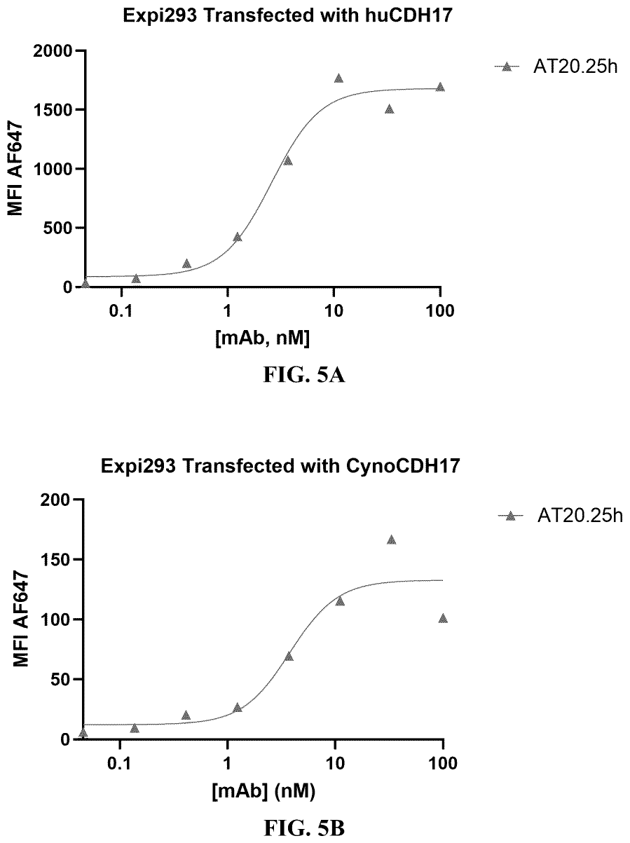

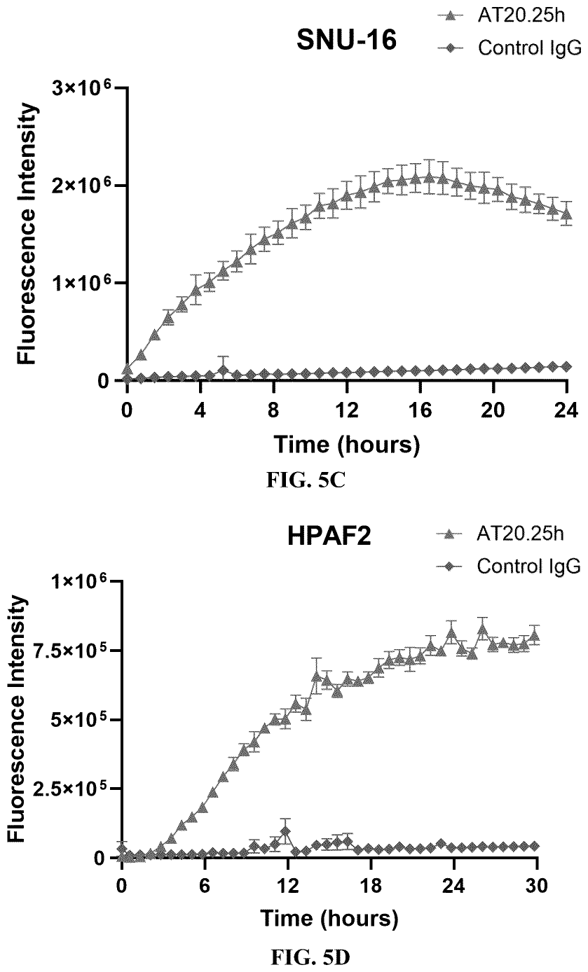

FIG. 5A shows high affinity cell surface binding of humanized AT-20.25 antibody (with AF647 secondary antibody) to Expi293 cells transfected with a human CDH17 expression plasmid so as to cause surface expression of human CDH17 on the transfected Expi293 cells. An EC50 value for AT20.25h binding to transfected Expi293 cells was calculated to be 2.6 nanomolar (nM). FIG. 5B shows high affinity binding of humanized AT-20.25 antibody (with AF647 secondary antibody) to Expi293 cells transfected with a cynomolgus CDH17 expression plasmid so as to cause surface expression of cynomolgus CDH17 on the transfected Expi293 cells. An EC50 value for AT20.25h binding to transfected Expi293 cells was calculated to be 3.8 nM. These figures illustrate that humanized AT-20 family antibodies bind to cell lines expressing human or cynomolgus cadherin-17 (CDH17) with EC50 values below 5 nM.

FIG. 5C and FIG. 5D show internalization kinetics for humanized AT-20.25 (AT-20.25h) anti-CDH17 antibody by SNU-16 gastric and HPAF2 pancreatic tumor cell lines, respectively. Cells were incubated with 25 nanomolar (nM) AT-20.25h antibody or IgG negative control, and internalization was measured using an Incucyte-based assay using secondary antibodies labeled with Fabfluor-pH Red pH-sensitive dye. Increases in detected fluorescence indicate internalization. AT-20.25h antibody reached 50% of maximum internalization (as determined by maximum measured fluorescence) at approximately 4 hours of incubation in SNU-16 cells and at about 9 hours of incubation in HPAF2 cells. IgG control experiments did not produce an appreciable fluorescent signal, suggesting that the IgG was not internalized. In contrast, the strong fluorescence detected for AT-20.25h experiments suggest that AT-20.25h is readily internalized by gastrointestinal tumor cells, which implies that AT-20.25h antibody-drug conjugates would be effective method of delivering antineoplastic drugs and payloads to tumor cells for the purpose of killing the tumor cells.

### Example 4: ADC-Based Cell Viability Assay

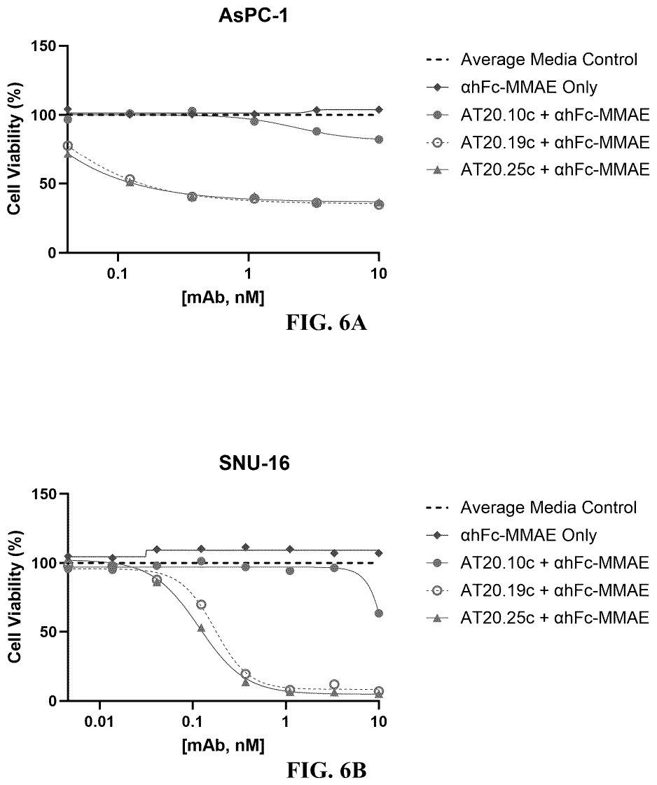

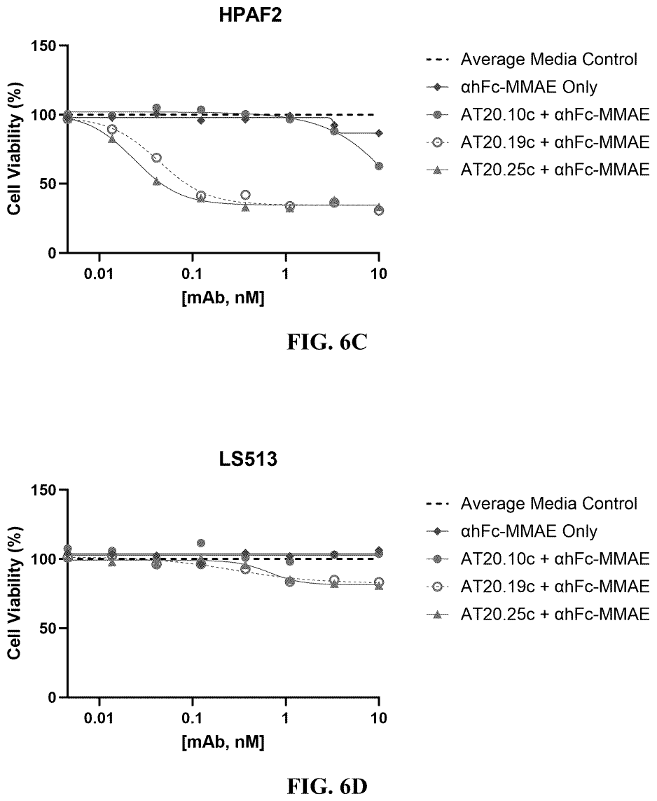

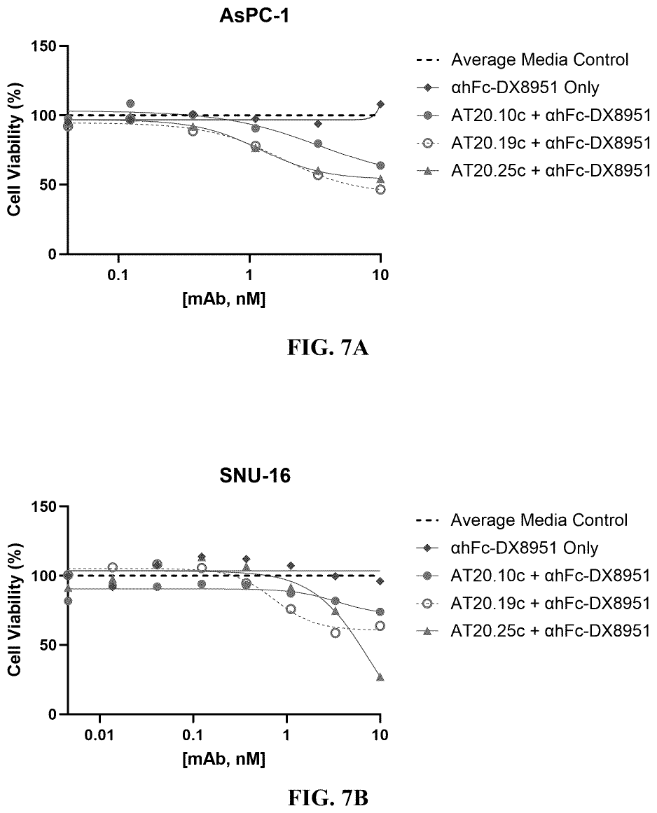

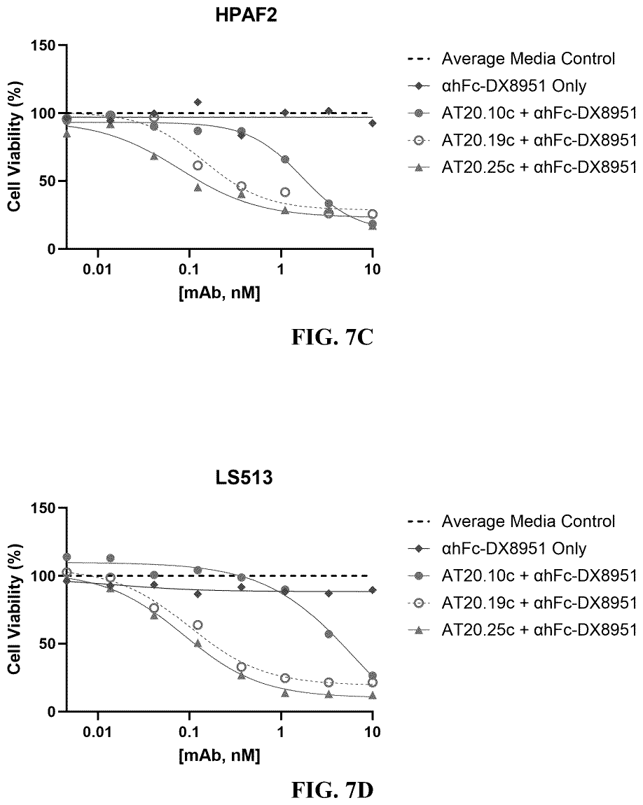

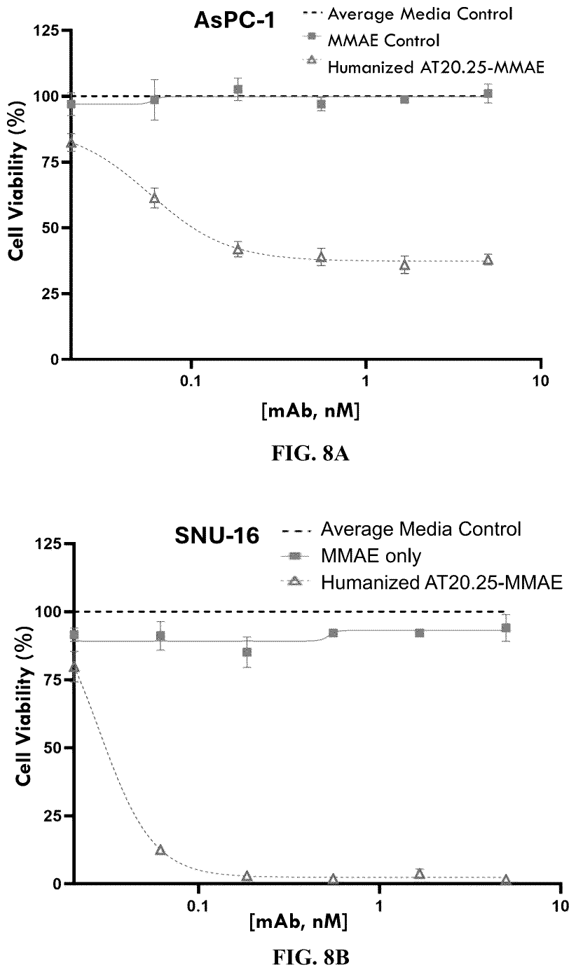

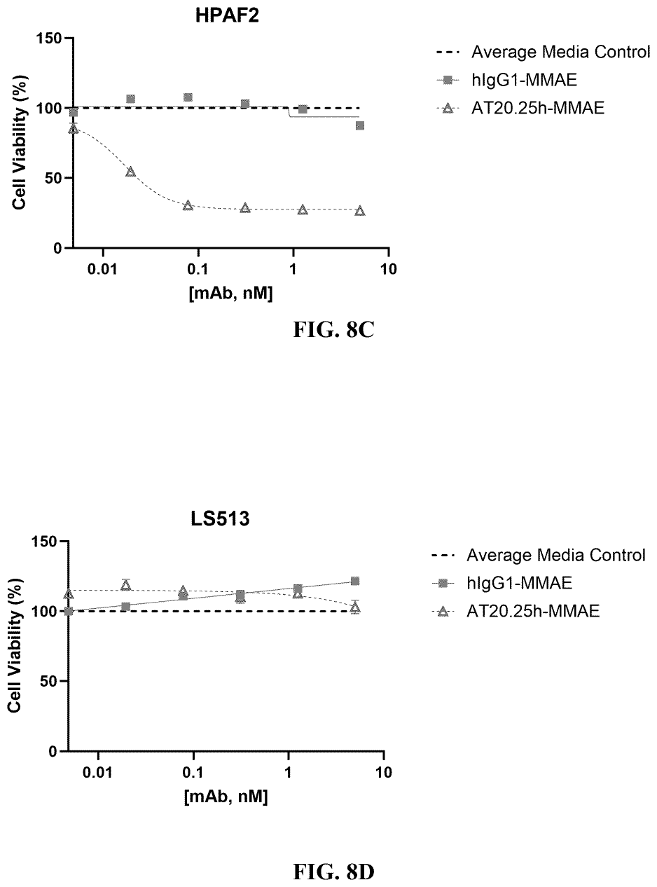

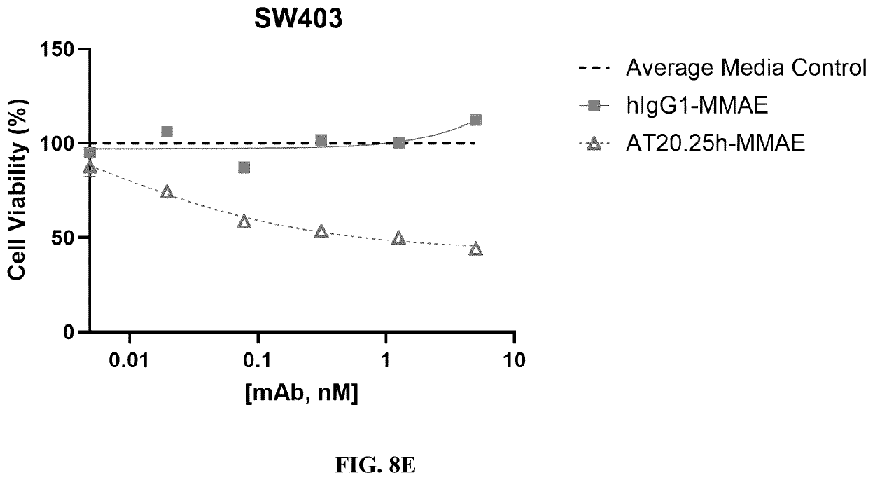

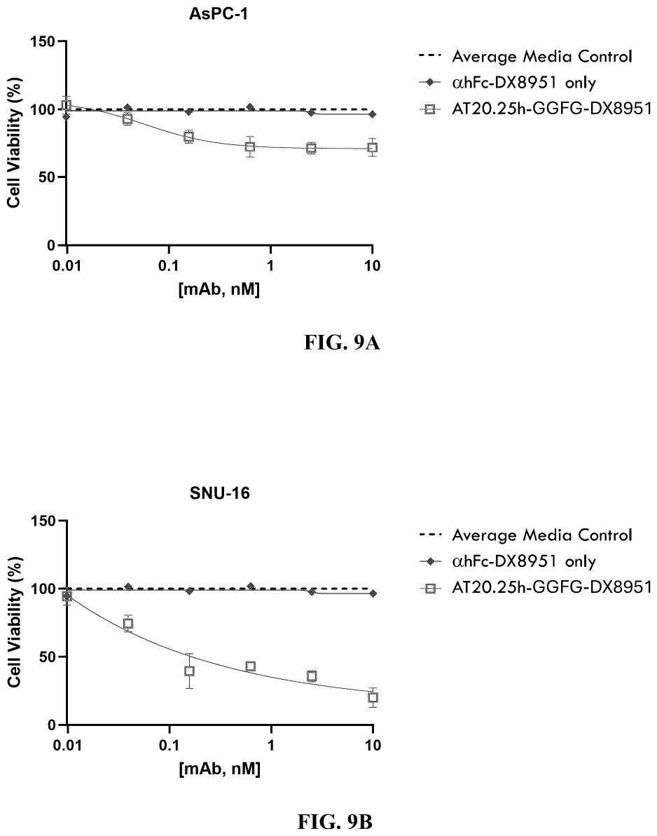

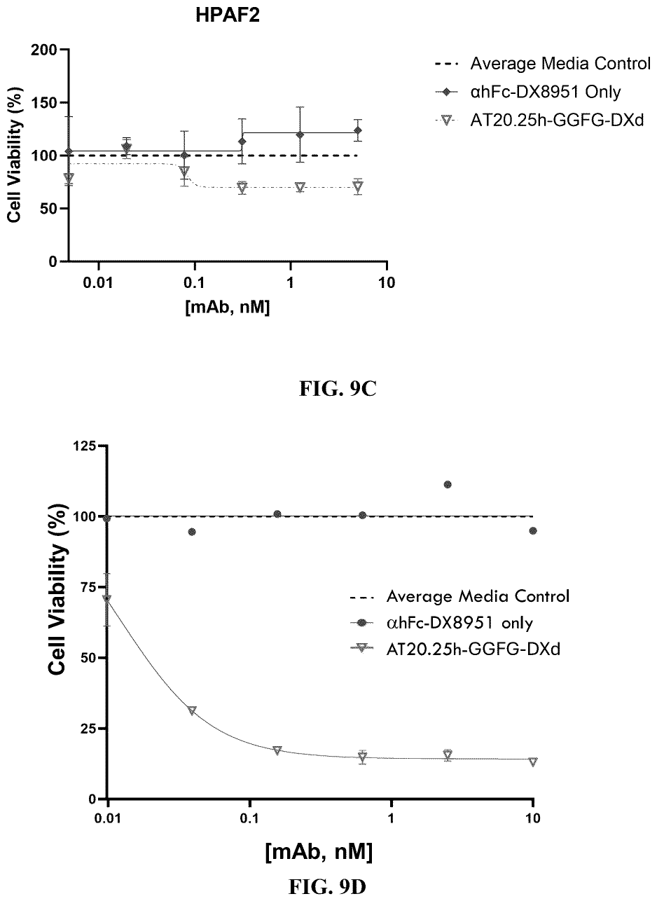

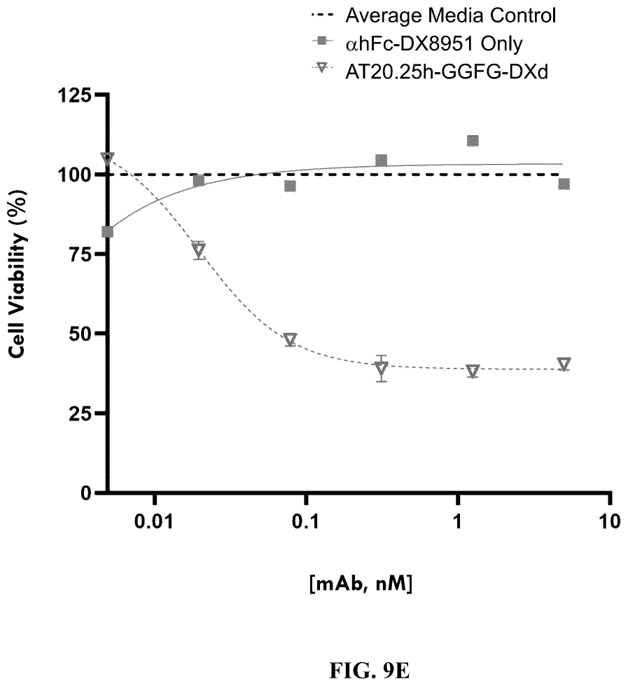

FIGS. 6A-6D, 7A-7D, 8A-8E, and 9A-9E show illustrative data modeling ADC effects of chimeric AT-20 antibodies bound by a secondary antibody conjugated to monomethyl auristatin E (αhFc-MMAE) or DX8951 (αhFc-DX8951), and humanized AT-20.25 antibody directly conjugated to MMAE or Deruxtecan (GGFG-DXd) across multiple CDH17 expressing tumor cell lines. Prior to the assay, AsPC-1 (FIG. 6A, FIG. 7A, FIG. 8A, FIG. 9A), SNU-16 (FIG. 6B, FIG. 7B, FIG. 8B, FIG. 9B), HPAF2 (FIG. 6C, FIG. 7C, FIG. 8C, FIG. 9C), LS513 (FIG. 6D, FIG. 7D, FIG. 8D, FIG. 9D) or SW403 human colorectal adenocarcinoma cells (FIG. 8E, FIG. 9E) cells were seeded in a white flat bottom 96-well plate at 2,500 cells/well (MMAE assay) or 1,000 cells/well (DX8951 or DXd assay). AT-20 chimeric antibodies were mixed with a toxin conjugated secondary antibody αhFc-CL-MMAE or αhFc-CL-DX8951 (Moradec) at a 1:2 ratio, and then added to the cells. Plates were incubated in a 37° C., 5% CO₂ incubator for 96 hours (MMAE assay) or 144 hours (DX8951 or DXd assay). After incubation, luminescence-based cell viability was measured using the CellTiter Glo2 (Promega) reagent as per manufacturer's instructions. Data is normalized to the average of media only treated cells. Humanized AT-20.25 antibody directly conjugated to either MMAE or GGFG-DXd induced decreases in tumor cell viability, e.g., as shown in FIGS. 6A, 6B, 6C, 6D, 7A, 7B, 7C, 7D, 8A, 8B, 8C, 8E, 9A, 9B, 9C, 9D, and 9E. For example, cell viability was decreased by about 95% compared to media control treatment in SNU-16 cells treated with chimeric AT-20.25h-MMAE antibody (FIG. 6B; IC50, 0.17 nM for AT20.19c+MMAE and EC50, 0.12 nM for AT20.25c+MMAE) by about 98% compared to media control treatment in SNU-16 cells treated with humanized AT-20.25h-MMAE antibody (FIG. 8B; IC50, 0.03 nM), by about 62% in AsPC-1 cells treated with humanized AT-20.25h-MMAE (FIG. 8A; IC50, 0.06 nM), by about 85% in LS-513 cells treated with humanized AT-20.25h-GGFG-DX8951 (FIG. 9D; IC50, 0.06 nM), and by about 60% in SW403 colorectal cancer treated with AT20.25-GGFG DXd (FIG. 9E; IC50, 0.02 nM). These results suggest that AT-20 assets disclosed herein (including humanized AT-20 assets, such as AT-20.25h) can decrease viability of tumor cells described herein (e.g., colorectal cancer, gastric cancer, pancreatic cancer, stomach cancer, and other gastrointestinal tumors and cancers) by up to 98%, up to 95%, up to 90%, up to 85%, up to 80%, up to 75%, up to 70%, up to 65%, up to 60%, up to 55%, up to 50%, up to 40%, up to 35%, up to 30%, up to 25%, up to 20%, up to 15%, up to 10%, up to 5%, at least 98%, at least 95%, at least 90%, at least 85%, at least 80%, at least 75%, at least 70%, at least 65%, at least 60%, at least 55%, at least 50%, at least 40%, at least 35%, at least 30%, at least 25%, at least 20%, at least 15%, at least 10%, at least 5%, from 5% to 98%, from 10% to 98%, from 15% to 98%, from 20% to 98%, from 25% to 98%, from 30% to 98%, from 35% to 98%, from 40% to 98%, from 45% to 98%, from 50% to 98%, from 55% to 98%, from 60% to 98%, from 65% to 98%, from 70% to 98%, from 75% to 98%, from 80% to 98%, from 65% to 98%, from 70% to 98%, from 75% to 98%, from 80% to 98%, from 85% to 98%, from 90% to 98%, from 95% to 98%, from 5% to 95%, from 10% to 95%, from 15% to 95%, from 20% to 95%, from 25% to 95%, from 30% to 95%, from 35% to 95%, from 40% to 95%, from 45% to 95%, from 50% to 95%, from 55% to 95%, from 60% to 95%, from 65% to 95%, from 70% to 95%, from 75% to 95%, from 80% to 95%, from 65% to 95%, from 70% to 95%, from 75% to 95%, from 80% to 95%, from 85% to 95%, from 90% to 95%, from 5% to 90%, from 10% to 90%, from 15% to 90%, from 20% to 90%, from 25% to 90%, from 30% to 90%, from 35% to 90%, from 40% to 90%, from 45% to 90%, from 50% to 90%, from 55% to 90%, from 60% to 90%, from 65% to 90%, from 70% to 90%, from 75% to 90%, from 80% to 90%, from 65% to 90%, from 70% to 90%, from 75% to 90%, from 80% to 90%, from 85% to 90%, from 5% to 80%, from 10% to 80%, from 15% to 80%, from 20% to 80%, from 25% to 80%, from 30% to 80%, from 35% to 80%, from 40% to 80%, from 45% to 80%, from 50% to 80%, from 55% to 80%, from 60% to 80%, from 65% to 80%, from 70% to 80%, from 75% to 80%, from 5% to 70%, from 10% to 70%, from 15% to 70%, from 20% to 70%, from 25% to 70%, from 30% to 70%, from 35% to 70%, from 40% to 70%, from 45% to 70%, from 50% to 70%, from 55% to 70%, from 60% to 70%, from 65% to 70%, from 5% to 60%, from 10% to 60%, from 15% to 60%, from 20% to 60%, from 25% to 60%, from 30% to 60%, from 35% to 60%, from 40% to 60%, from 50% to 60%, from 55% to 60%, from 5% to 50%, from 10% to 50%, from 15% to 50%, from 20% to 50%, from 25% to 50%, from 30% to 50%, from 35% to 50%, from 40% to 50%, from 45% to 50%, from 5% to 40%, from 10% to 40%, from 15% to 40%, from 20% to 40%, from 25% to 40%, from 30% to 40%, from 35% to 40%, from 5% to 30%, from 10% to 30%, from 15% to 30%, from 20% to 30%, from 25% to 30%, from 5% to 20%, from 10% to 20%, from 15% to 20%, or from 5% to 10%.

### Example 5: ADC-Based Cell Viability Assay Using Secondary Antibody Conjugated to MMAE Via a Cleavable Linker

FIGS. 6A-6D show experimental data illustrating effects of chimeric AT-20 antibodies (AT-20.10c, AT-20.19c, and AT-20.25c) antibodies bound by a secondary antibody conjugated to monomethyl auristatin E (MMAE) in AsPC-1 pancreatic tumor cells, SNU-16 gastric tumor cells, HPAF2 pancreatic tumor cells, and LS513 colorectal tumor cells, respectively. Data is normalized to the average viability of media only treated cells (dashed line). These figures show that AT-20 chimeric antibodies bound to an anti-human Fc secondary antibody conjugated to MMAE via a cleavable linker decrease cell viability compared to media treated control cells and cells treated with the secondary antibody only. The effects on cell viability are most pronounced with the AT-20.19c and AT-20.25c antibodies, with IC50 values lower than 1 nM. IC50 values for AT20.10c with αhFc-MMAE secondary antibody and payload, AT20.19c with αhFc-MMAE secondary antibody and payload, and AT20.25c with αhFc-MMAE secondary antibody and payload inhibition of AsPC-1 cell viability were calculated to be 2.3 nanomolar (nM), 0.01 nM, and 0.003 nM, respectively (FIG. 6A). Maximum reduction in AsPC-1 cell viability was calculated to be 18% for AT20.10c+αhFc-MMAE, 65% for AT20.19c+αhFc-MMAE, and 64% for AT20.25c+αhFc-MMAE compared to media-only treated negative controls (dashed line). No reduction in cell viability was found after negative control treatment of AsPC-1 cells with secondary antibody conjugated to MMAE payload only. IC50 values for AT20.19c with αhFc-MMAE secondary antibody and payload, and AT20.25c with αhFc-MMAE secondary antibody and payload inhibition of SNU-16 cell viability were calculated to be 0.17 nanomolar (nM) and 0.12 nM, respectively (FIG. 6B). Maximum reduction in SNU-16 cell viability was calculated to be 37% for AT20.10c+αhFc-MMAE, 93% for AT20.19c+αhFc-MMAE, and 94% for AT20.25c+αhFc-MMAE compared to media-only treated negative controls (dashed line). No reduction in cell viability was found after negative control treatment of SNU-16 cells with secondary antibody conjugated to MMAE payload only. IC50 values for AT20.19c with αhFc-MMAE secondary antibody and payload, and AT20.25c with αhFc-MMAE secondary antibody and payload inhibition of HPAF2 cell viability were calculated to be 0.04 nanomolar (nM) and 0.02 nM, respectively (FIG. 6C). Maximum reduction in HPAF2 cell viability was calculated to be 37% for AT20.10c+αhFc-MMAE, 69% for AT20.19c+αhFc-MMAE, and 67% for AT20.25c+αhFc-MMAE compared to media-only treated negative controls (dashed line). No reduction in cell viability was found after negative control treatment of HPAF2 cells with secondary antibody conjugated to MMAE payload only. IC50 values for AT20.19c with αhFc-MMAE secondary antibody and payload, and AT20.25c with αhFc-MMAE secondary antibody and payload inhibition of LS513 cell viability were calculated to be 0.3 nanomolar (nM) and 0.7 nM, respectively (FIG. 6D). Maximum reduction in LS513 cell viability was calculated to be 17% for AT20.19c+αhFc-MMAE and 19% for AT20.25c+αhFc-MMAE compared to media-only treated negative controls (dashed line). No reduction in cell viability was found after negative control treatment of LS513 cells with secondary antibody conjugated to MMAE payload only.

### Example 6: ADC-Based Cell Viability Assay Using Secondary Antibody Conjugated to DX8951 Via a Cleavable Linker

FIGS. 7A-7D show experimental data illustrating effects of chimeric AT-20 antibodies (AT-20.10c, AT-20.19c, and AT-20.25c) antibodies bound by a secondary antibody conjugated to DX8951 in AsPC-1 pancreatic tumor cells, SNU-16 gastric tumor cells, HPAF2 pancreatic tumor cells, and LS513 colorectal tumor cells, respectively. Data is normalized to the average viability of media only treated cells (dashed line). These figures show that AT-20 chimeric antibodies bound to an anti-human Fc secondary antibody conjugated to DX8951 via a cleavable linker decrease cell viability compared to media treated control cells and cells treated with the secondary antibody only. IC50 values for AT20.10c with αhFc-DX8951 secondary antibody and payload, AT20.19c with αhFc-DX8951 secondary antibody and payload, and AT20.25c with αhFc-DX8951 secondary antibody and payload inhibition of AsPC-1 cell viability were calculated to be 3.4 nanomolar (nM), 1.8 nM, and 1.2 nM, respectively (FIG. 7A). Maximum reduction in AsPC-1 cell viability was calculated to be 36% for AT20.10c+αhFc-DX8951, 54% for AT20.19c+αhFc-DX8951, and 46% for AT20.25c+αhFc-DX8951 compared to media-only treated negative controls (dashed line). No reduction in cell viability was found after negative control treatment of AsPC-1 cells with only secondary antibody conjugated to DX8951 payload. IC50 values for AT20.10c with αhFc-DX8951 secondary antibody and payload, AT20.19c with αhFc-DX8951 secondary antibody and payload, and AT20.25c with αhFc-DX8951 secondary antibody and payload inhibition of SNU-16 cell viability were calculated to be 3.6 nanomolar (nM), 0.7 nM, and 7.8 nM, respectively (FIG. 7B). Maximum reduction in SNU-16 cell viability was calculated to be 26% for AT20.10c+αhFc-DX8951, 36% for AT20.19c+αhFc-DX8951, and 73% for AT20.25c+αhFc-DX8951 compared to media-only treated negative controls (dashed line). No reduction in cell viability was found after negative control treatment of SNU-16 cells with only secondary antibody conjugated to DX8951 payload. IC50 values for AT20.10c with αhFc-DX8951 secondary antibody and payload, AT20.19c with αhFc-DX8951 secondary antibody and payload, and AT20.25c with αhFc-DX8951 secondary antibody and payload inhibition of HPAF2 cell viability were calculated to be 1.7 nanomolar (nM), 0.14 nM, and 0.08 nM, respectively (FIG. 7C). Maximum reduction in HPAF2 cell viability was calculated to be 82% for AT20.10c+αhFc-DX8951, 74% for AT20.19c+αhFc-DX8951, and 83% for AT20.25c+αhFc-DX8951 compared to media-only treated negative controls (dashed line). No reduction in cell viability was found after negative control treatment of HPAF2 cells with only secondary antibody conjugated to DX8951 payload. IC50 values for AT20.10c with αhFc-DX8951 secondary antibody and payload, AT20.19c with αhFc-DX8951 secondary antibody and payload, and AT20.25c with αhFc-DX8951 secondary antibody and payload inhibition of LS513 cell viability were calculated to be 6.3 nanomolar (nM), 0.1 nM, and 0.09 nM, respectively (FIG. 7D). Maximum reduction in LS513 cell viability was calculated to be 74% for AT20.10c+αhFc-DX8951, 79% for AT20.19c+αhFc-DX8951, and 88% for AT20.25c+αhFc-DX8951 compared to media-only treated negative controls (dashed line). No reduction in cell viability was found after negative control treatment of LS513 cells with only secondary antibody conjugated to DX8951 payload.

### Example 7: ADC-Based Cell Viability Assay Using Humanized AT-20.25 Directly Conjugated to MMAE Via a Cleavable Linker

FIGS. 8A-8E show experimental data illustrating functional characteristics of humanized AT-20.25 antibody directly conjugated to MMAE via a cleavable linker in AsPC-1 pancreatic tumor cells, SNU-16 gastric tumor cells, HPAF2 pancreatic tumor cells, LS513 colorectal tumor cells, and SW403 colorectal tumor cells. Data is normalized to the average viability of media only treated cells (dashed line). These figures show that humanized AT-20.25 directly conjugated to MMAE via a cleavable linker decreases cell viability compared to media treated control cells and cells treated with the hIgG1-MMAE negative control (gray squares) with IC50 values lower than 0.1 nM in all cell lines tested except LS513 cells. An IC50 value for AT20.25h-MMAE inhibition of AsPC-1 cell viability was calculated to be 0.09 nanomolar (nM) (FIG. 8A). Maximum reduction in AsPC-1 cell viability was calculated to be 60% for AT20.25h-MMAE compared to media-only treated negative controls (dashed line). No reduction in cell viability was found after negative control treatment of AsPC-1 cells with human IgG1 conjugated to MMAE (hIgG1-MMAE). An IC50 value for AT20.25h-MMAE for inhibition of SNU-16 cell viability was calculated to be 0.02 nanomolar (nM) (FIG. 8B). Maximum reduction in SNU-16 cell viability was calculated to be 99% for AT20.25h-MMAE compared to media-only treated negative controls (dashed line). No reduction in cell viability was found after negative control treatment of SNU-16 cells with human IgG1 conjugated to MMAE (hIgG1-MMAE). An IC50 value for AT20.25h-MMAE inhibition of HPAF2 cell viability were calculated to be 0.02 nanomolar (nM) (FIG. 8C). Maximum reduction in HPAF2 cell viability were calculated to be 73% for AT20.25h-MMAE compared to media-only treated negative controls (dashed line). No reduction in cell viability was found after negative control treatment of HPAF2 cells with human IgG1 conjugated to MMAE (hIgG1-MMAE). No IC50 value for AT20.25h-MMAE inhibition of LS513 colorectal carcinoma cell viability was calculated (FIG. 8D; however, an IC50 value for AT20.25h-MMAE inhibition of SW403 human colorectal adenocarcinoma cell viability was calculated to be 0.005 nanomolar (nM) (FIG. 8E). Maximum reduction in SW403 cell viability was calculated to be 56% for AT20.25h-MMAE compared to media-only treated negative controls (dashed line). No reduction in cell viability was found after negative control treatment of SW403 cells with human IgG1 conjugated to MMAE (hIgG1-MMAE).

### Example 8: ADC-Based Cell Viability Assay Using Humanized AT-20.25 Directly Conjugated to Deruxtecan (DXd) Via a Cleavable Linker

FIGS. 9A-9E show experimental data illustrating functional characteristics of humanized AT-20.25 antibody directly conjugated to Exatecan (DX8951) via a cleavable tetrapeptide GGFG linker or to Deruxtecan (DXd) via a cleavable tetrapeptide GGFG linker in AsPC-1 pancreatic tumor cells, SNU-16 gastric tumor cells, HPAF2 pancreatic tumor cells, LS513 colorectal tumor cells, and SW403 colorectal tumor cells. Data is normalized to the average viability of media only treated cells (dashed line). These figures show that humanized AT-20.25 directly conjugated to DXd via a cleavable linker decreases cell viability compared to media treated control cells and cells treated with the αhFc-DX8951 negative control (red diamonds). An IC50 value for AT20.25h-GGFG-DX8951 (AT20.25h-GGFG-DX8951) inhibition of AsPC-1 cell viability was calculated to be 0.06 nanomolar (nM) (FIG. 9A). Maximum reduction in AsPC-1 cell viability was calculated to be 28% for AT20.25h-GGFG-DX8951 compared to media-only treated negative controls (dashed line). No reduction in cell viability was found after negative control treatment of AsPC-1 cells with anti-human Fc antibody conjugated to DX8951 (αhFc-DX8951). An IC50 value for AT20.25h-GGFG-DX8951 inhibition of SNU-16 cell viability was not calculated from the data shown in FIG. 9B; however, the maximum reduction of cell viability was calculated to be 80%, compared to treatment with anti-human Fc antibody conjugated to DX8951 (αhFc-DX8951). An IC50 value for AT20.25h-GGFG-DX8951 inhibition of HPAF2 cell viability was calculated to be 0.08 nanomolar (nM) (FIG. 9C). Maximum reduction in HPAF2 cell viability was calculated to be 30% for AT20.25h-GGFG-DX8951 compared to media-only treated negative controls (dashed line). No reduction in cell viability was found after negative control treatment of HPAF2 cells with anti-human Fc antibody conjugated to DX8951 (αhFc-DX8951). An IC50 value for AT20.25h-GGFG-DX8951 inhibition of LS513 cell viability were calculated to be 0.06 nanomolar (nM) (FIG. 9D). Maximum reduction in LS513 cell viability was calculated to be 64% for AT20.25h-GGFG-DX8951 compared to media-only treated negative controls (dashed line). No reduction in cell viability was found after negative control treatment of LS513 cells with anti-human Fc antibody conjugated to DX8951 (αhFc-DX8951). An IC50 value for AT20.25h-GGFG-DX8951 inhibition of SW403 cell viability were calculated to be 0.03 nanomolar (nM) (FIG. 9E). Maximum reduction in SW403 cell viability was calculated to be 43% for AT20.25h-GGFG-DX8951 compared to media-only treated negative controls (dashed line). No reduction in cell viability was found after negative control treatment of SW403 cells with anti-human Fc antibody conjugated to DX8951 (αhFc-DX8951).

### Example 9: ADC-Based Reduction of SNU-16 Gastric Tumor Volume In Vivo

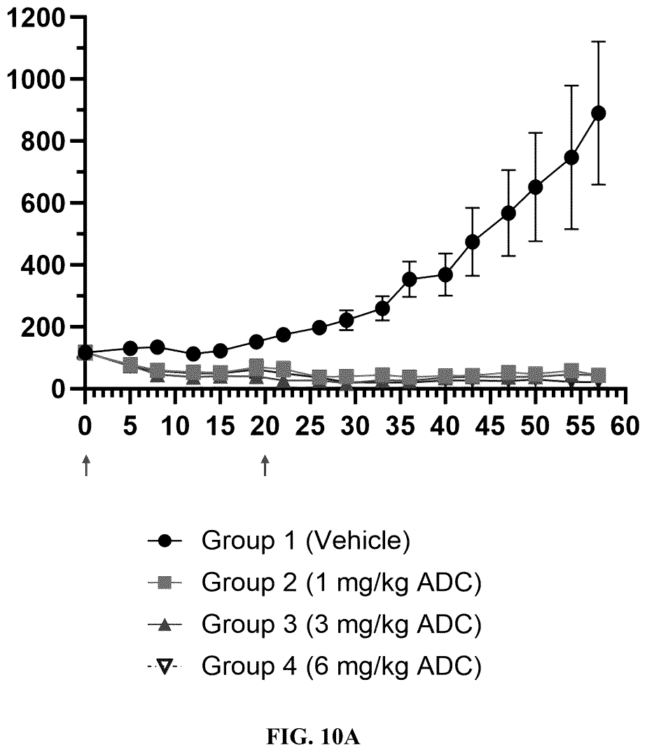

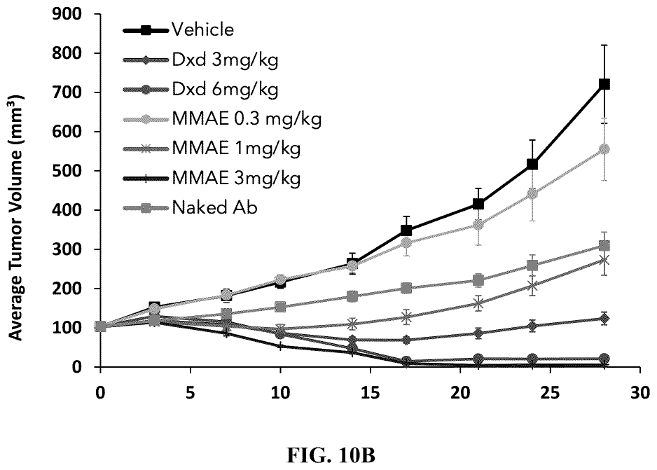

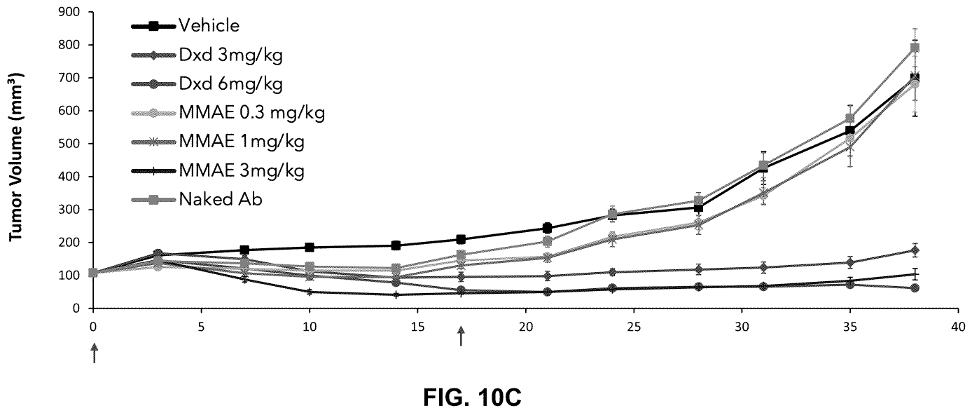

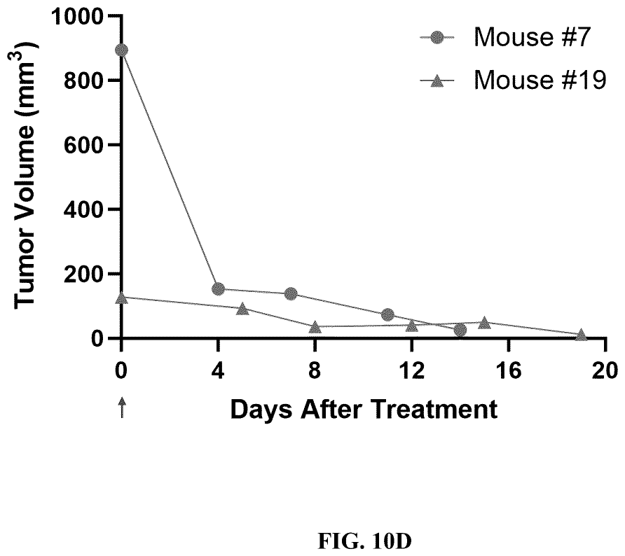

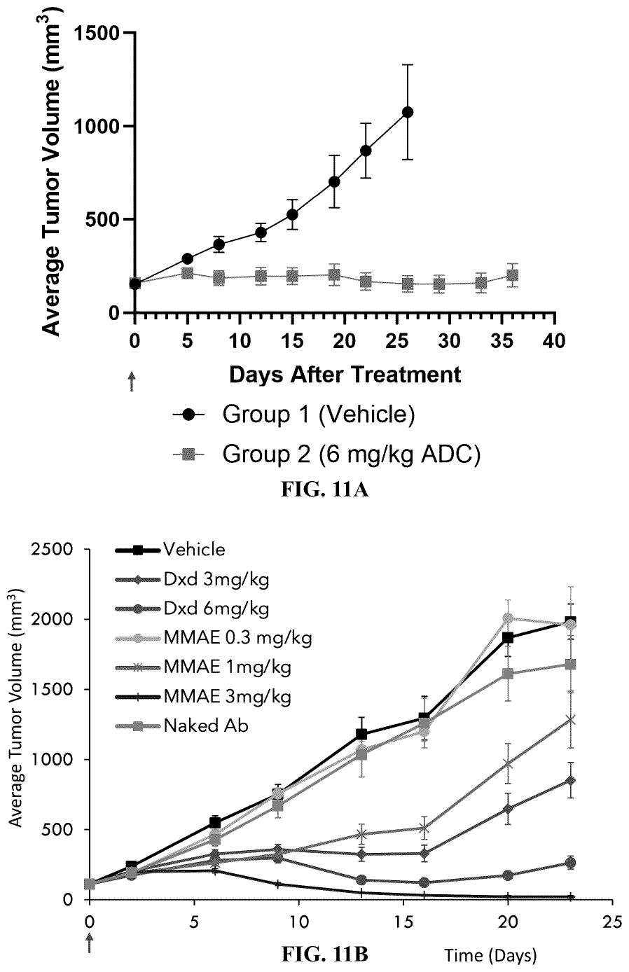

FIGS. 10A, 10B, and 10C show experimental data illustrating the effect of humanized AT-20.25 antibody directly conjugated to MMAE via a cleavable linker on SNU-16 gastric tumor volume in Balb/C nude mice. FIG. 10A shows experimental data illustrating that humanized AT-20.25 antibody directly conjugated to MMAE via a cleavable linker can reduce SNU-16 tumor volume at various dosages. AT-20.25h antibody conjugated to MMAE via a cleavable linker delivered in one injection at day 0 and one injection at day 20 reduced SNU-16 gastric tumor volume in Balb/C nude mice for at least 36 days. As shown in FIG. 10A, tumors in vehicle treated mice (Group 1, filled black circles) grew to approximately three times the initial tumor size over the 36 day period, whereas tumor size decreased in mice treated with 1 mg/kg of AT-20.25h antibody-drug conjugate (filled green squares), 3 mg/kg of AT-20.25h antibody-drug conjugate (filled green triangles pointing up), and 6 mg/kg of AT 20.25h (dark green upside-down open triangles with dashed line) antibody-drug conjugate (wherein the drug conjugate is MMAE) by day 7 and remained reduced through the 36 day experiment. This data shows that gastric tumor volume reduction can be achieved at doses as low as 1 mg/kg during in vivo experiments. FIG. 10B shows tumor size over time in nude Balb/C mice seeded subcutaneously with 100 mm³ SNU-16 tumors after a single injection (red arrow) at time 0 with 3 mg/kg of humanized AT-20.25 antibody (AT-20.25h) conjugated to DXd payload with GGFG cleavable linker (red diamonds), with 6 mg/kg of humanized AT-20.25 antibody (AT-20.25h) conjugated to DXd payload with GGFG cleavable linker (green circles), with 0.3 mg/kg of humanized AT-20.25 antibody (AT-20.25h) conjugated to MMAE payload with MC-VC-PAB cleavable linker (orange circles), with 1 mg/kg of humanized AT-20.25 antibody (AT-20.25h) conjugated to MMAE payload with MC-VC-PAB cleavable linker (gray asterisks), with 3 mg/kg of humanized AT-20.25 antibody (AT-20.25h) conjugated to MMAE payload with MC-VC-PAB cleavable linker (blue plus signs), with naked (i.e., no payload) humanized AT-20.25 antibody (AT-20.25h) (gray squares), or with vehicle control solution (black squares). Results show that injection of AT-20.25h antibody-drug conjugates attenuated tumor growth over time in all cases, with AT-20.25h-GGFG-DXd (3 mg/kg and 6 mg/kg) and AT-20.25h-MC-VC-PAB-MMAE (3 mg/kg) producing the most substantial decreases in tumor growth. Indeed, AT-20.25h-GGFG-DXd (6 mg/kg) and AT-20.25h-MC-VC-PAB-MMAE (3 mg/kg) treatment actually decreased tumor size by the end of the experiment. Interestingly, naked AT-20.25h antibody decreased tumor growth compared to vehicle control, raising the possibility that the antibody can act through an antibody-dependent cellular cytotoxicity (ADCC) mechanism. Data are presented as mean tumor volume±SEM. FIG. 10C shows tumor size over time in nude Balb/C mice seeded subcutaneously with 100 mm³ SNU-16 tumors after a single injection at day 0 and a single injection at day 17 (red arrows) with 3 mg/kg of humanized AT-20.25 antibody (AT-20.25h) conjugated to DXd payload with GGFG cleavable linker (red diamonds), with 6 mg/kg of humanized AT-20.25 antibody (AT-20.25h) conjugated to DXd payload with GGFG cleavable linker (green circles), with 0.3 mg/kg of humanized AT-20.25 antibody (AT-20.25h) conjugated to MMAE payload with MC-VC-PAB cleavable linker (orange circles), with 1 mg/kg of humanized AT-20.25 antibody (AT-20.25h) conjugated to MMAE payload with MC-VC-PAB cleavable linker (gray asterisks), with 3 mg/kg of humanized AT-20.25 antibody (AT-20.25h) conjugated to MMAE payload with MC-VC-PAB cleavable linker (blue plus signs), with naked (i.e., no payload) humanized AT-20.25 antibody (AT-20.25h) (gray squares), or with vehicle control solution (black squares). Results show that injection of AT-20.25h antibody-drug conjugates attenuated tumor growth overtime in all cases, with AT-20.25h-GGFG-DXd (3 mg/kg and 6 mg/kg) and AT-20.25h-MC-VC-PAB-MMAE (3 mg/kg) producing the most substantial decreases in tumor growth. Indeed, AT-20.25h-GGFG-DXd (6 mg/kg) and AT-20.25h-MC-VC-PAB-MMAE (3 mg/kg) treatment actually decreased tumor size by the end of the experiment. Naked AT-20.25h antibody decreased tumor growth compared to vehicle control until at least day 21, raising the possibility that the antibody can act through an antibody-dependent cellular cytotoxicity (ADCC) mechanism. Data are presented as mean tumor volume±SEM. FIG. 10D shows that a single injection of humanized AT-20.25h ADC with an MMAE payload conjugated by a cleavable linker (GGFG) at day 0 (indicated by arrow in FIG. 10C) was sufficient to reduce tumor size in Balb/C nude mice having large initial volumes (approximately 900 mm³; “Mouse #7”, circles) or small initial volume (approximately 150 mm³; “Mouse #19”, triangles) in less than 4 days and less than 5 days, respectively, with advantageous tumor volume reduction persisting until at least 14 days and 19 days, respectively.

### Example 10: ADC-Based Inhibition of AsPC-1 Pancreatic Tumor Growth In Vivo

FIG. 11A shows experimental data illustrating the effect of humanized AT-20.25 antibody directly conjugated to MMAE via a cleavable linker (MC-Val-Cit-PAB) on AsPC-1 tumor volume in Balb/C nude mice. AT-20.25h antibody conjugated to MMAE via a cleavable linker delivered in one injection on day 0 (indicated by arrow) inhibited AsPC-1 tumor growth in Balb/C nude mice for at least 36 days. As shown in FIG. 11A, tumors in vehicle treated mice (Group 1, black circles) grew to an average tumor volume of approximately 1000 mm³ over the course of 26 days, whereas tumors in mice treated with 6 mg/kg of AT-20.25h antibody-drug conjugate (green squares) showed substantial inhibition of tumor growth, as evaluated by average tumor volume over the course of 36 days. Data are presented as mean tumor volume±SEM. FIG. 11B shows tumor size over time in nude Balb/C mice seeded subcutaneously with 100 mm³ HPAF2 tumors after a single injection at day 0 (red arrow) with 3 mg/kg of humanized AT-20.25 antibody (AT-20.25h) conjugated to DXd payload with GGFG cleavable linker (red diamonds), with 6 mg/kg of humanized AT-20.25 antibody (AT-20.25h) conjugated to DXd payload with GGFG cleavable linker (green circles), with 0.3 mg/kg of humanized AT-20.25 antibody (AT-20.25h) conjugated to MMAE payload with MC-VC-PAB cleavable linker (orange circles), with 1 mg/kg of humanized AT-20.25 antibody (AT-20.25h) conjugated to MMAE payload with MC-VC-PAB cleavable linker (gray asterisks), with 3 mg/kg of humanized AT-20.25 antibody (AT-20.25h) conjugated to MMAE payload with MC-VC-PAB cleavable linker (blue plus signs), with naked (i.e., no payload) humanized AT-20.25 antibody (AT-20.25h) (gray squares), or with vehicle control solution (black squares). Results suggest that injection of AT-20.25h antibody-drug conjugates attenuated tumor growth over time in all cases by Day 23, with AT-20.25h-GGFG-DXd (3 mg/kg and 6 mg/kg) and AT-20.25h-MC-VC-PAB-MMAE (3 mg/kg) producing the most substantial decreases in tumor growth. Indeed, AT-20.25h-GGFG-DXd (6 mg/kg) and AT-20.25h-MC-VC-PAB-MMAE (3 mg/kg) treatment actually decreased tumor size by the end of the experiment. Naked AT-20.25h antibody also decreased tumor growth compared to vehicle control, raising the possibility that the antibody can act through an antibody-dependent cellular cytotoxicity (ADCC) mechanism. Data are presented as mean tumor volume±SEM.

### Example 11: CDH17 Domain Binding ELISA

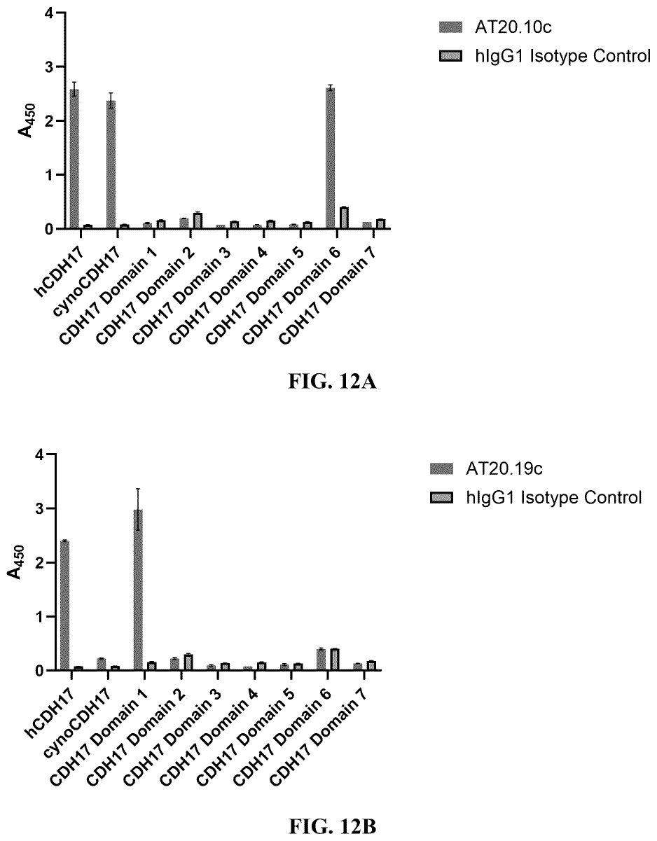

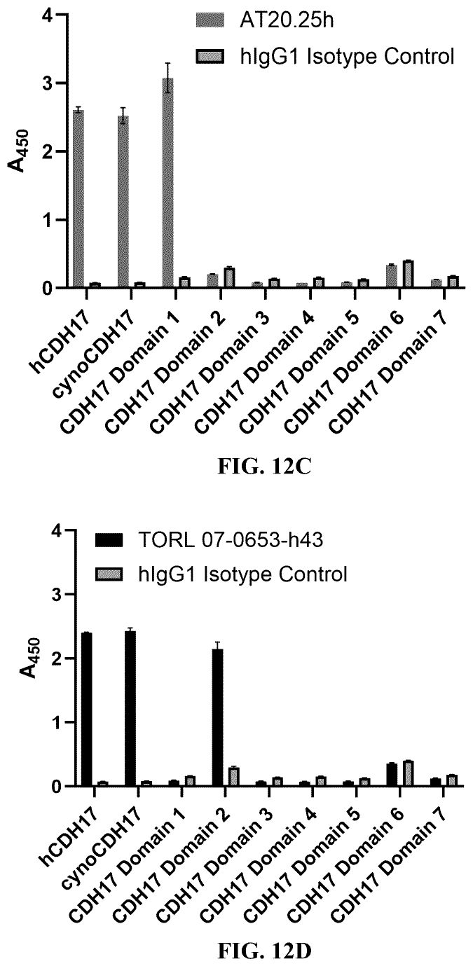

FIG. 12A-12C show experimental data illustrating the domain on CDH17 bound by AT-20 family anti-cadherin 17 antibodies described herein. 96-well high-binding ELISA Plates (NEST) were respectively coated with recombinant human CDH17 (Sino Biological), recombinant cynomolgus CDH17 (Sino Biological), and CDH17 domains 1-7 (KACTUS) at a concentration of 1 microgram per milliliter (g/mL). Chimeric AT-20.10c, chimeric AT-20.19c, humanized AT-20.25h, or an hIgG1 isotype negative control were incubated on coated wells at a concentration of 100 nM. Bound antibody was detected with a goat-anti human Fc-HRP secondary antibody, using TMB substrate (3,3′,5,5′-tetramethylbenzidine) and stop solution (2N Sulfuric Acid). Absorbance at 450 nm was obtained using a microplate reader. As shown in FIG. 12A, AT-20.10c exhibits strong positive binding to recombinant human CDH17 and cynomolgus CDH17 and was found to preferentially bind to CDH17 domain 6, as compared to CDH17 domains 1, 2, 3, 4, 5, and 7 in these experiments. Only baseline binding of hIgG1 isotype control antibody was observed for each tested condition. As shown in FIG. 12B, AT-20.19c exhibits strong positive binding to recombinant human CDH17 but not to recombinant cynomolgus CDH17. AT-20.19c binds to CDH17 domain 1 but shows only baseline signal when tested for binding to CDH17 domains 2, 3, 4, 5, 6, and 7. Only baseline binding of hIgG1 isotype control antibody was observed for each tested condition. As shown in FIG. 12C, AT-20.25h exhibits strong positive binding to recombinant human and cynomolgus CDH17. AT-20.25h binds to CDH17 domain 1 but shows only baseline signal when tested for binding to CDH17 domains 2, 3, 4, 5, 6, and 7. Only baseline binding of hIgG1 isotype control antibody was observed for each tested condition. FIG. 12D shows levels of binding of TORL 07-0653-h43 to human CDH17, cynomolgus CDH17, and each of CDH17 domains 1-7. As shown, TORL 07-0653-h43 binds to human and cynomolgus CDH17 and to domain 2 of CDH17, but not to domains 1, 3, 4, 5, 6, and 7 of CDH17, suggesting that this antibody may have a different mechanism of action and/or efficacy as AT-20.25h, which binds to CDH17 domain 1, AT-20.19c, which binds to CDH domain 1, and AT-20.10c, which binds to CDH domain 6. Only baseline binding of hIgG1 isotype control was observed for each tested condition.

### Example 12: Cadherin Family Antibody Cross-Reactivity Assay

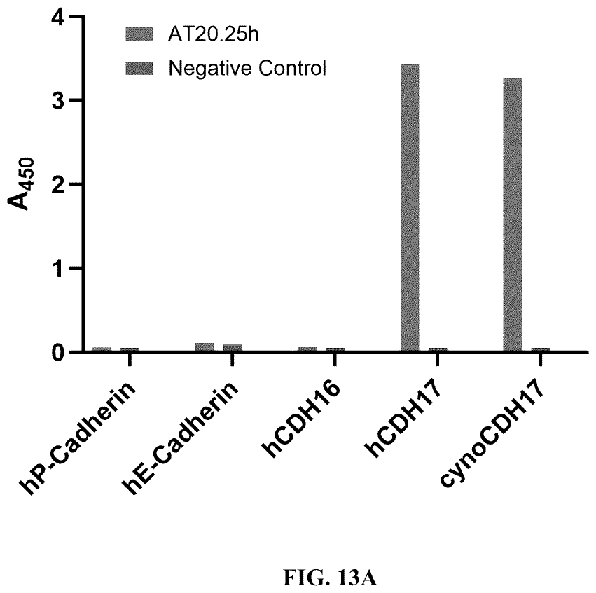

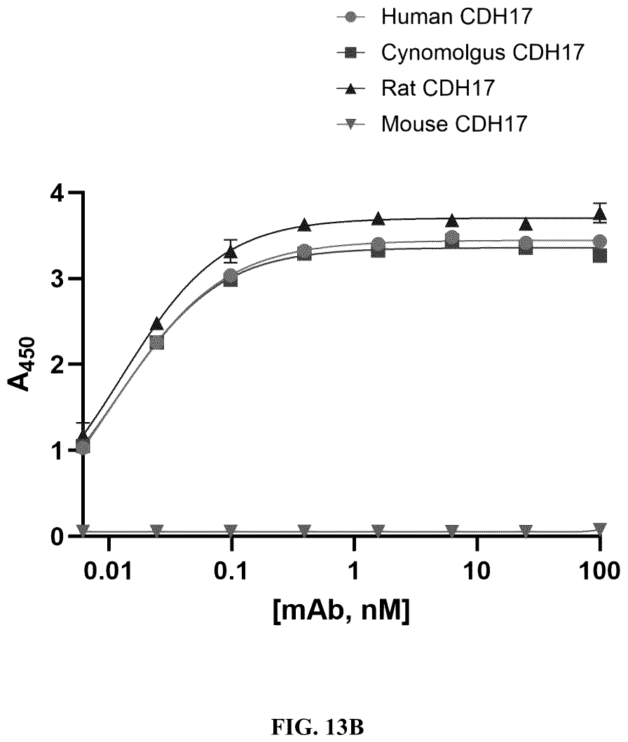

FIG. 13A shows experimental data illustrating that humanized AT-20.25 exhibits no cross-reactivity to other cadherin family proteins. Humanized AT-20.25 antibodies at a concentration of 100 nM, or negative vehicle controls were incubated on a coated surface of 1 microgram per milliliter (g/mL) of human P-Cadherin (hP-Cadherin), human E-Cadherin (hE-Cadherin), human Cadherin-16 (hCDH16), human Cadherin-17 (hCDH17), or cynomolgus Cadherin-17 (cynoCDH17). Bound antibody was detected with a goat-anti human Fc-HRP secondary antibody, using TMB substrate and stop Solution (2N Sulfuric Acid). Absorbance at 450 nm was obtained using a microplate reader. The AT-25.20h antibody shows positive binding against hCDH17 and cynoCDH17, with only background levels of binding to hP-Cadherin, hE-Cadherin, and hCDH16. This suggests that the AT20.25h antibody shows specific binding to cadherin-17 and not to other human cadherin types.

FIG. 13B shows experimental data illustrating that humanized AT-20.25 binds to human, cynomolgus, and rat cadherin 17 (CDH17) but not to mouse CDH17. Humanized AT-20.25 antibodies were incubated on a coated surface of 1 microgram per milliliter (g/mL) of human cadherin-17 (Human CDH17, circles), cynomolgus cadherin-17 (Cynomolgus CDH17, squares), rat cadherin-17 (Rat CDH17), upward triangles) or mouse cadherin-17 (Mouse CDH17). Bound antibody was detected with a goat-anti human Fc-HRP secondary antibody, using TMB substrate and stop Solution (2N Sulfuric Acid). Absorbance at 450 nm (A₄₅₀) was obtained using a microplate reader. The AT-25.20h antibody shows positive binding against human CDH17 (EC50 value: 0.011 nanomolar (nM)), cynomolgus CDH17 (EC50 value: 0.012 nM), and rat CDH17 (EC50 value: 0.013). Only background binding was observed for mouse CDH17 for the entire tested concentration range. This suggests that the AT-20.25h antibody can bind human, cynomolgus, and rat cadherin-17 protein.

### Example 13: Surface Plasmon Resonance (SPR) Analysis of Humanized AT-20.25 Binding to huCDH17

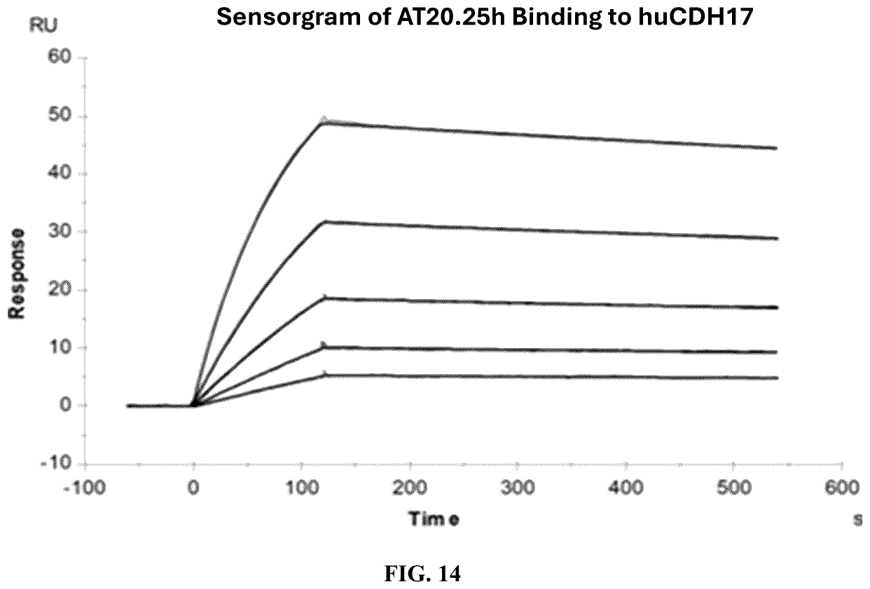

FIG. 14 represents the sensorgram of AT-20.25h binding to various concentrations of recombinant human CDH17 (12.5-200 nM). SPR experiments were conducted using a BIAcore T200 system (Cytiva) to determine the binding kinetics and affinity of AT-20.25h antibody to Human CDH17, His Tag protein. Protein A was immobilized to a CM5 chip that was activated via NHS-EDC chemistry to a conjugation level of ˜5200 RU, followed by blocking of unreacted sites with ethanolamine. The running buffer used was 1×HBS-EP+(10 mM HEPES, 150 mM NaCl, 3 mM EDTA, 0.05% surfactant P20, pH 7.4). AT-20.25h was diluted to 1 g/mL and injected for 25 s for Protein A capture. The analyte, Human CDH17, His Tag, was injected at 12.5-200 nM concentration range at a flow rate of 30 μL/min at 25° C. for 120 s (association phase), followed by a 420 s dissociation phase and regeneration of the chip. The binding kinetics were analyzed using BIAcore T200 Evaluation Software (version 3.2) under a 1:1 binding model. The association rate constant (ka), dissociation rate constant (kd), and equilibrium dissociation constant (KD) were determined. SPR analysis showed that AT-20.25h exhibits high-affinity, specific binding to huCDH17, with a calculated KD (equilibrium dissociation constant) of 3.89 nM. Calculated association rate constant (ka) for AT-20.25h was calculated to be 57130 inverse molar per second (1/Ms), and dissociation rate constant (kd) for AT-20.25h was calculated to be 2.220×10⁻⁴ inverse seconds (1/s). By comparison, TORL-0663 anti-CDH17 antibody was found to have an equilibrium dissociation constant (KD) of 1.73 nM, a calculated association rate constant (ka) of 19480 inverse molar per second (1/Ms), and a calculated dissociation rate constant (kd) of 3.368×10⁻⁴ inverse seconds (1/s), suggesting that the AT-20.25h exhibits a slower off rate than the TORL-0663 antibody.

### Example 14: Antibody-Dependent Cellular Cytotoxicity Assays

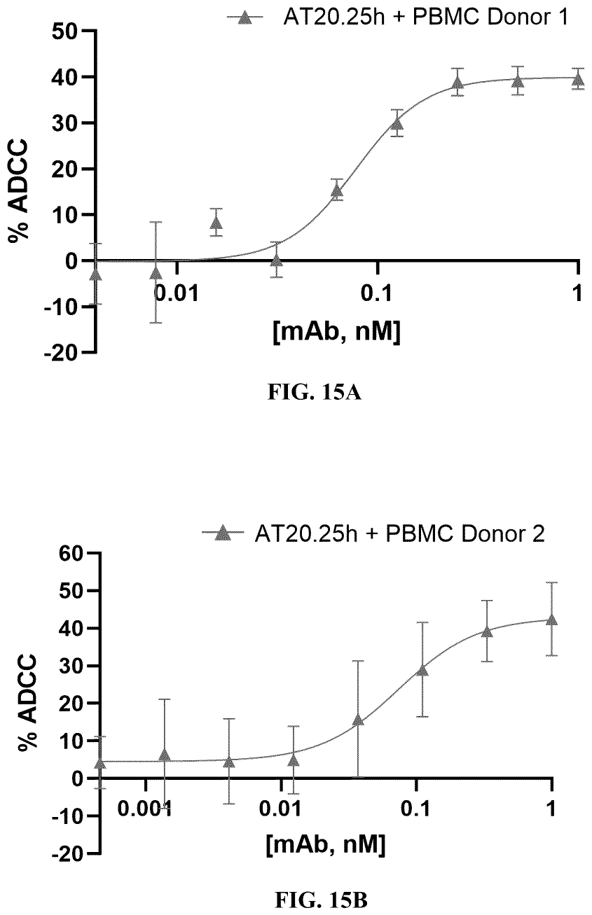

FIG. 15A and FIG. 15B show antibody-dependent cellular cytotoxicity assay results for humanized AT-20.25 antibody (AT-20.25h) in the presence of peripheral blood mononuclear cells (PBMCs) as measured in the context of SNU-C1 colorectal tumor cell viability. FIG. 15A was performed with PBMCs derived from a first human donor, whereas FIG. 15B was performed with PBMCs derived from a second human donor. All data is expressed as percentage decrease in live SNU-C1 cells in vitro. EC50 value for FIG. 15A was calculated to be 0.08 nM, with a maximum SNU-C1 viability decrease of 40%. EC50 value for FIG. 15B was calculated to be 0.02 nM, with a maximum SNU-C1 viability decrease of 42%. These data provide evidence that AT-20 assets (including humanized AT-20.25 antibodies) can be used to reduce the growth of gastrointestinal tumor and cancer cells (including colorectal tumors and cancers) independent of antineoplastic agent payload (e.g., wherein antibodies are administered or used as a “naked” antibody without a payload attached), for example, via an antibody-dependent cellular cytotoxicity mechanism.

### Example 15: In Vitro Immunogenicity Assays

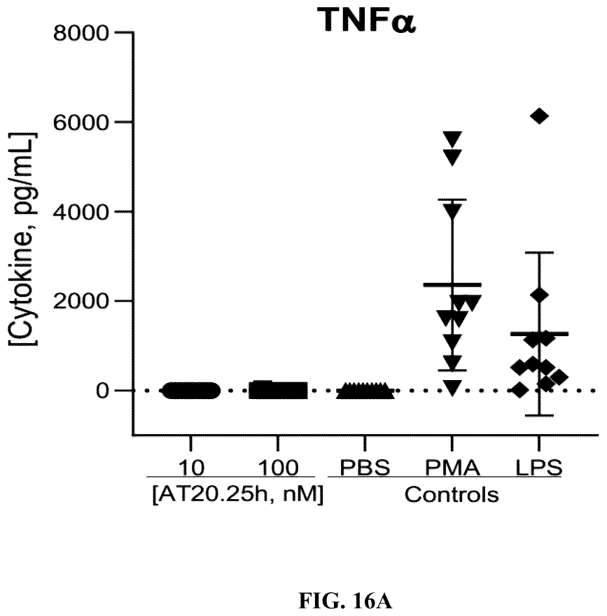

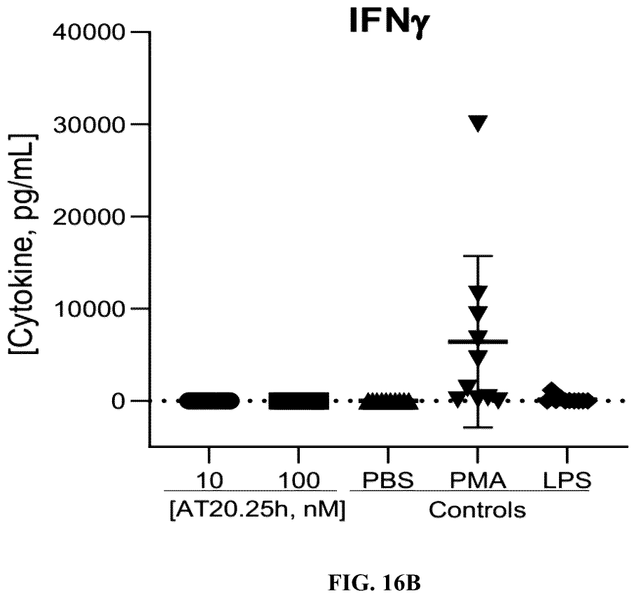

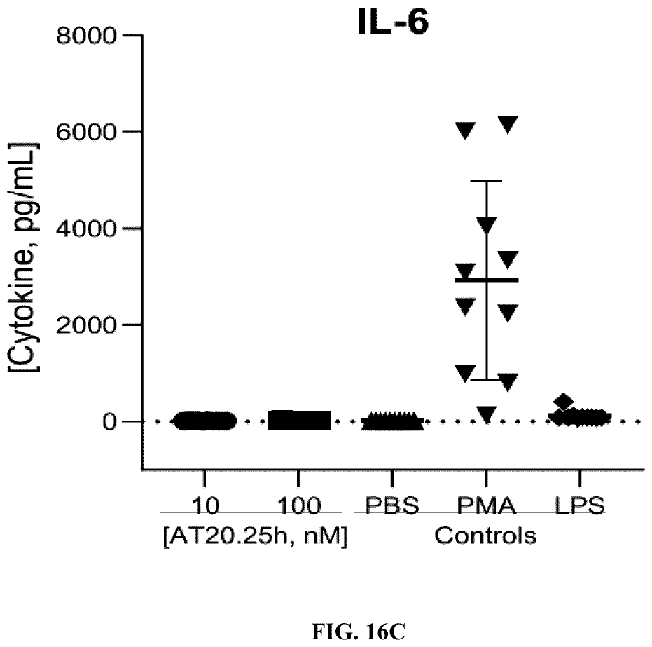

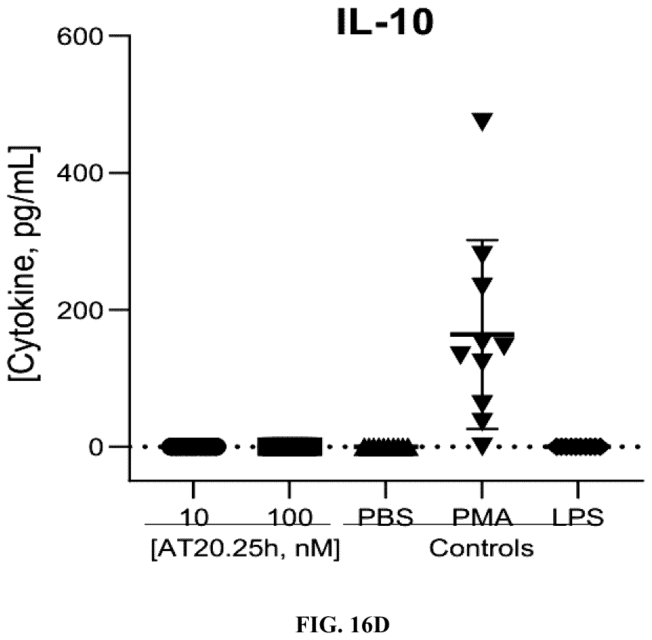

FIGS. 16A-16D show data from in vitro immunogenicity experiments in which human peripheral blood mononuclear cells (PBMCs) obtained from 10 healthy donors were treated with 10 nanomolar (nM) or 100 nM of humanized AT-20.25 (AT-20.25h) antibody, with 50 ng/ml of phorbol-12-myristate-13-acetate (PMA), with 5 micrograms per milliliter (g/ml) lipopolysaccharide (LPS), or with phosphate-buffered saline (PBS) control for 16 hours. Secretion of inflammatory cytokines tumor necrosis factor alpha (TNFα), interferon gamma (IFNγ), interleukin 6 (IL-6), and interleukin 10 (IL-10) were measured using meso-scale discovery (MSD) assay kits. Neither 10 nM nor 100 nM of AT-20.25h antibody induced secretion of TNFα (FIG. 16A), IFNγ (FIG. 16B), IL-6 (FIG. 16C), or IL-10 (FIG. 16D) inflammatory cytokines from PBMCs, indicating that AT-20.25h does not induce proinflammatory cytokine release from PBMCs. Positive control PMA treatment induced secretion of TNFα, IFNγ, IL-6, and IL-10 by PBMCs, as shown in FIGS. 16A-16D. Positive control LPS treatment induced secretion of TNFα but not IFNγ, IL-6, or IL-10. Negative PBS-treated groups showed no secretion of any of the four tested inflammatory cytokines (see third column in FIGS. 16A-16D).

### Example 16: In Vitro Polyreactivity Assays

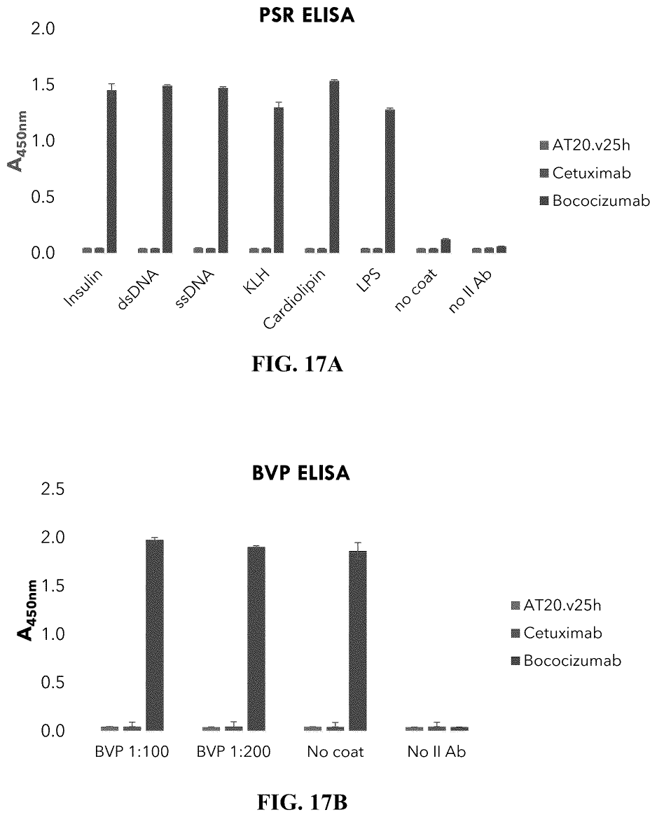

FIGS. 17A and 17B show data from in vitro polyreactivity experiments. In FIG. 17A, humanized AT-20.25 (AT-20.v25h) antibody binding to various polyspecificity reagents (PSRs) (insulin, double-stranded DNA (dsDNA), single-stranded DNA (ssDNA), keyhole limpet hemocyanin (KLH), cardiolipin, or lipopolysaccharide (LPS)) and were detected using a secondary antibody conjugated to a detectable fluorophore in an enzyme-linked immunosorbent assay (ELISA). AT-20.25h antibody (leftmost bar for each condition) showed only background levels of binding to each of the tested PSRs, indicating no interaction between AT-20.25h and the tested PSRs. Negative control antibody Cetuximab (middle bar for each condition) also showed only background signal for each tested PSR. Positive control Bococizumab (rightmost bar for each condition) showed strong detectable signal for each PSR, indicating association with the PSRs. Only background signal levels were detected for each of AT-20.25h antibody, cetuximab and bococizumab when tested without a PSR coating on the plate (“no coat”) or when no fluorescent secondary antibody was used (“no II Ab”). FIG. 17B shows results from baculovirus particle (BVP) ELISA assays, wherein BVP was coated onto assay plates at either 1:100 or 1:200 concentrations before AT20.25h antibody, cetuximab, or bococizumab were added and incubated with a fluorescent secondary antibody. No fluorescent signal was detected for assays wherein AT-20.25h antibody was incubated with 1:100 or 1:200 BVP, suggesting no interaction between the two. Data also showed no fluorescent signal when cetuximab was incubated on 1:100 or 1:200 BVP. Neither AT-20.25h nor cetuximab showed a fluorescent signal to when incubated on uncoated plates (“no coat”) or when the fluorescent secondary antibody was not added (“No II Ab”). A strong fluorescent signal was observed for bococizumab binding to 1:100 BVP and 1:200 BVP conditions.

### Example 17: Plasma and Thermal Stability Assays

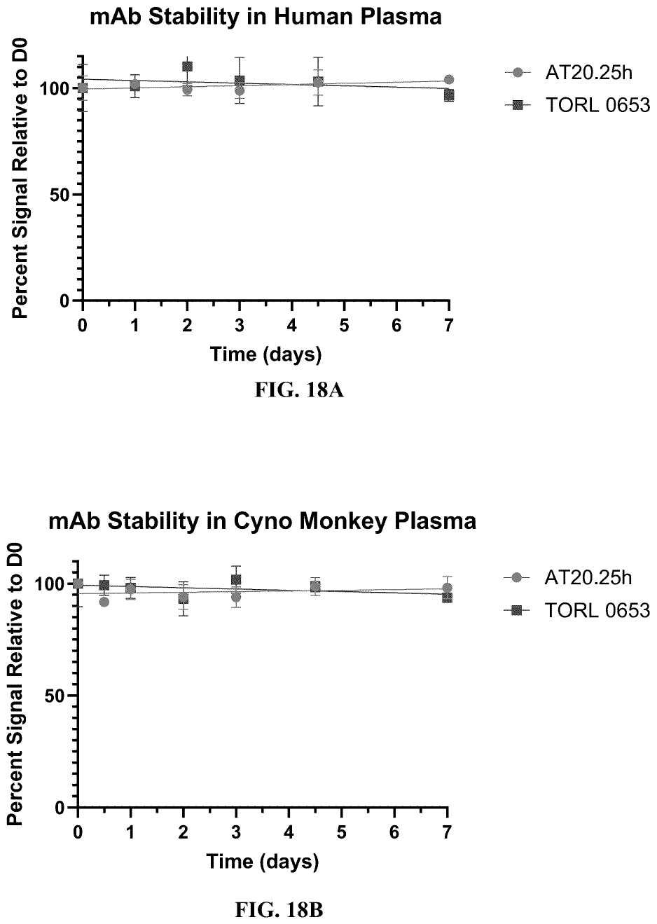

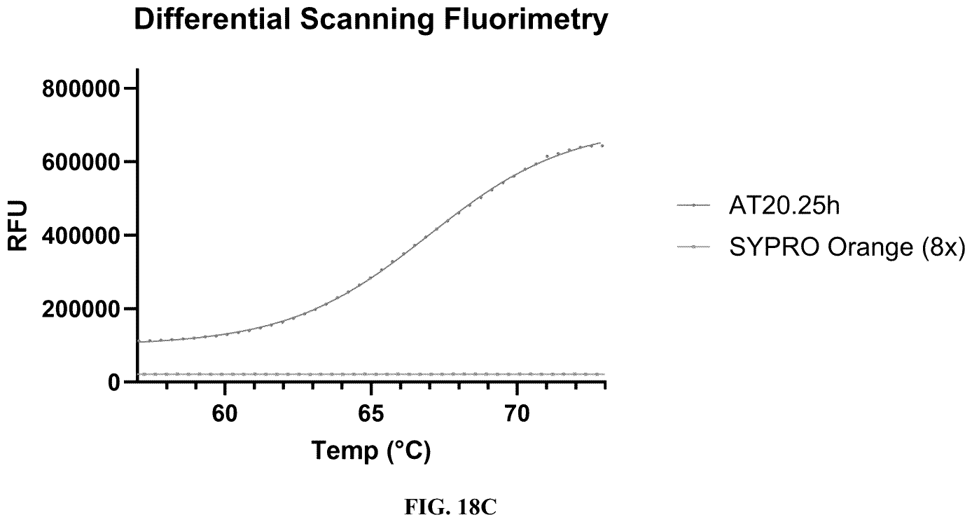

FIGS. 18A-18B show data from in vitro plasma stability experiments in which humanized AT-20.25 (AT-20.25h) antibody (green circles) or TORL-07-0653-h43 anti-CDH17 antibody (red squares) was incubated in human plasma (FIG. 18A) or cynomolgus plasma (FIG. 18B) for 7 days at 37° C. and monitored with ELISA. Results showed that AT-20.25h antibody was highly stable in human plasma and cynomolgus plasma, suggesting that the antibody maintains its structure and function in vivo. FIG. 18C shows experimental in vitro data showing that AT-20.25h antibody (without conjugated payload) exhibits robust thermal stability, with a melting temperature of 67.0° C., implying that AT-20 antibody assets will maintain structure and function in vivo at body temperatures.

### Example 18: ADC-based Reduction of Colorectal Cancer Patient-Derived Xenograft Tumor Volume In Vivo

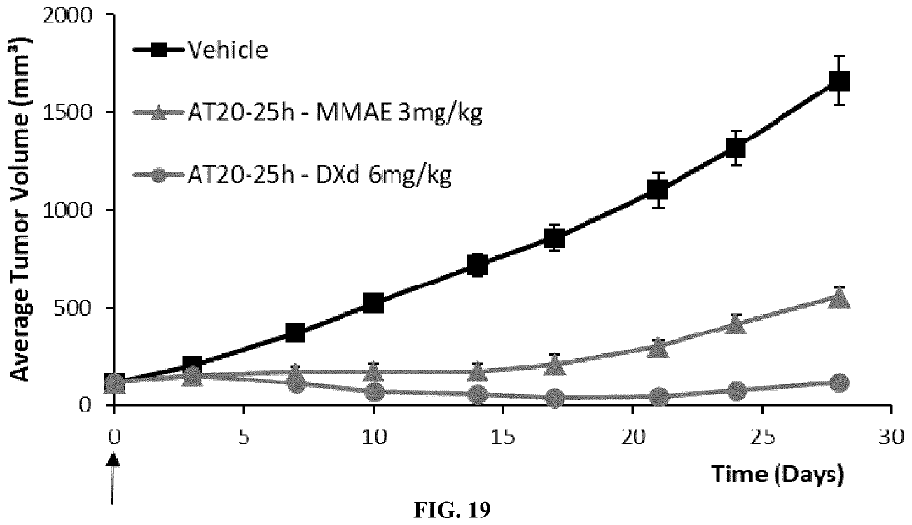

FIG. 19 shows experimental data illustrating the effect of humanized AT20-25 (AT20-25h) antibody drug conjugates on CR5055 colorectal cancer patient-derived xenograft (PDX) tumor volume in NOD/SCID mice. AT20-25h antibodies were directly conjugated to MMAE (3 mg/kg, gray triangles) or DXd (6 mg/kg, gray circles) via a cleavable linker. To generate the data shown in FIG. 19, a VC-PAB linker was used for conjugating MMAE to AT20-25h antibody to create antibody drug conjugate AT20-25h—VC-PAB-MMAE (gray triangles), and a GGFG linker was used for conjugating DXd to AT20-20h antibody create antibody drug conjugate AT20-25h—GGFG-DXd (gray circles). These experimental data show that humanized AT-20.25 antibody directly conjugated to MMAE (e.g., AT20-25h—VC-PAB-MMAE) or DXd (e.g., AT20-25h—GGFG-DXd) via a cleavable linker can reduce CR5055 tumor volume compared to vehicle controls (black squares). AT-20.25h antibody conjugated to MMAE or DXd via a cleavable linker was delivered in one injection at day 0 (FIG. 19, arrow) abrogated CR5055 colorectal cancer tumor volume growth in NOD/SCID nude mice for at least 28 days. As shown in FIG. 19, tumors in vehicle (PBS) treated mice (black squares) grew to approximately ten times the initial tumor size over the 28-day period. In mice treated with 3 mg/kg of AT-20.25h antibody-drug conjugate with MMAE payload (gray triangles), the final tumor volume at day 28 was approximately one-third that of vehicle-treated xenograft tumors. In mice treated with 6 mg/kg of AT-20.25h antibody-drug conjugate (Group 3, filled grey circle) wherein the drug conjugate is DXd, the tumor size decreased by approximately 2.5-fold relative to the initial tumor measurement at DO. Indeed, AT-20.25h-GGFG-DXd (6 mg/kg) treatment actually decreased tumor size from the initial tumor volume at DO by day 21. Data are presented as mean tumor volume±SEM.

### Example 19: Clinical Treatment Paradigms

FIG. 20 shows clinical treatment paradigms in which AT-20 antibody assets (e.g., antibodies comprising one or more sequence listed in Table 1, including humanized or chimeric versions of AT-20.10, AT-20.19, and AT-20.25 antibodies or antibodies including portions of the sequences listed in Table 1 thereof) are used to treat a subject having or at risk of having a tumor or cancer (e.g., a gastrointestinal cancer or tumor, including a colorectal cancer or tumor, a pancreatic cancer or tumor, an esophageal cancer or tumor, a gastric cancer or tumor). FIG. 20 shows potential first line (1L), second line (2L) third line (3L) or fourth line or later (4L+) treatments that can be used with one or more AT-20 antibodies. For some patients, an AT-20 antibody asset (e.g., humanized or chimeric AT-20.25 antibody) is administered along with one or more treatment listed in FIG. 20. For example, an AT-20 antibody asset is administered in some cases (e.g., as a first line treatment) in patients with microsatellite instability-high (MSI-H) presentation and/or deficient mismatch repair (dMMR) presentation (e.g., in biomarker-dependent tumors and cancers). In some regimens, the AT-20 antibody (e.g., as an AT-20 antibody ADC described herein or as a naked AT-20 antibody) is administered with FOLFOX, FOLFIRI, or FOLFOX-IRI chemotherapy regimens, e.g., along with vascular endothelial growth factor inhibitors (VEGFi). As shown in FIG. 20, such treatments are sometimes used in biomarker independent cases, for example, along with AT-20 antibody cotreatment (e.g., as an AT-20 antibody ADC described herein or as a naked AT-20 antibody). Regimens outlined in FIG. 20 include treatment with trifluridine, tipiracil and VEGFi, or with Irinotecan with epidermal growth factor receptor inhibitors (EGFRi), or with regorafenib, or with fruquinitinib. As contemplated in FIG. 20 but not shown, AT-20 antibodies (e.g., an AT-20 antibody ADC described herein or a naked AT-20 antibody) are used in regimens for treating a subject having or at risk of having a cancer or tumor (e.g., a gastrointestinal cancer or tumor, including a colorectal cancer or tumor, a pancreatic cancer or tumor, an esophageal cancer or tumor, a gastric cancer or tumor) in place of one or more treatment options shown in FIG. 20 for the patient population indicated in in FIG. 20 (e.g., biomarker dependent cancers and tumors with MSI-H and/or dMMR or biomarker independent cancers and tumors) or in conjunction with a listed treatment (e.g., FOLFOX, FOLFIRI, FOLFOX-IRI, VEGFi, EGFRi, irinotecan, trifluiridine, tipiracil, fruquinitib, or regorafenib).

TABLES
Table 1: Sequences according to embodiments of AT-20 Assets (e.g., anti-CDH17 antibodies)

| SEQ ID | Asset | Type | Chain | Region | Sequence |
|---|---|---|---|---|---|
| 1 | AT-20.10 | protein | Light | CDR L1 | IASQSVDNYGISFMN |
| 2 | AT-20.10 | protein | Light | CDR L2 | AASNQGS |
| 3 | AT-20.10 | protein | Light | CDR L3 | QQSKEIPLT |
| 4 | AT-20.10 | protein | Heavy | CDR H1 | TQWMN |
| 5 | AT-20.10 | protein | Heavy | CDR H2 | QIFPASGSTYYNEMFKD |
| 6 | AT-20.10 | protein | Heavy | CDR H3 | RGGSGMDY |
| 7 | AT-20.10 | protein | Light | Variable | DIVLTQSPASLAVSLRHRATISC |
| | | | | | IASQSVDNYGISFMNWFQHKP |
| | | | | | GQPPKLLIYAASNQGSGVPARF |
| | | | | | SGSGSGTDFSLNIHPMEEDDTA |
| | | | | | MYFCQQSKEIPLTFGAGTKLELK |
| 8 | AT-20.10 | protein | Heavy | Variable | QVQLQQSGPDLVRPGTSVKISC |
| | | | | | KGSGYTFLTQWMNWVKQRPG |
| | | | | | QGLEWIGQIFPASGSTYYNEMF |
| | | | | | KDKATLTVDTSSSTAYMQLSSL |
| | | | | | TSEDTAVYFCARRGGSGMDY |
| | | | | | WGQGTSVTVSS |
| 9 | AT-20.10c | protein | Light | CDR L1 | IASQSVDNYGISFMN |
| 10 | AT-20.10c | protein | Light | CDR L2 | AASNQGS |
| 11 | AT-20.10c | protein | Light | CDR L3 | QQSKEIPLT |
| 12 | AT-20.10c | protein | Heavy | CDR H1 | TQWMN |
| 13 | AT-20.10c | protein | Heavy | CDR H2 | QIFPASGSTYYNEMFKD |
| 14 | AT-20.10c | protein | Heavy | CDR H3 | RGGSGMDY |
| 15 | AT-20.10c | protein | Light | Variable | DIVLTQSPASLAVSLRHRATISC |
| | | | | | IASQSVDNYGISFMNWFQHKP |
| | | | | | GQPPKLLIYAASNQGSGVPARF |
| | | | | | SGSGSGTDFSLNIHPMEEDDTA |
| | | | | | MYFCQQSKEIPLTFGAGTKLELK |
| 16 | AT-20.10c | protein | Heavy | Variable | QVQLQQSGPDLVRPGTSVKISC |
| | | | | | KGSGYTFLTQWMNWVKQRPG |
| | | | | | QGLEWIGQIFPASGSTYYNEMF |
| | | | | | KDKATLTVDTSSSTAYMQLSSL |
| | | | | | TSEDTAVYFCARRGGSGMDY |
| | | | | | WGQGTSVTVSS |
| 17 | AT-20.19 | protein | Light | CDR LI | RASQGIRGNLD |
| 18 | AT-20.19 | protein | Light | CDR L2 | STSNLNS |
| 19 | AT-20.19 | protein | Light | CDR L3 | LQRNAYPLT |
| 20 | AT-20.19 | protein | Heavy | CDR H1 | DYHIN |
| 21 | AT-20.19 | protein | Heavy | CDR H2 | WIFPGSGSTYYNEKFKG |
| 22 | AT-20.19 | protein | Heavy | CDR H3 | GKSGYPFYFDY |
| 23 | AT-20.19 | protein | Light | Variable | DIQMIQSPSSMFASLGDRVSLS |
| | | | | | CRASQGIRGNLDWYQQKPGG |
| | | | | | TIKLLIYSTSNLNSGVPSRFSGS |
| | | | | | GSGSDYSLTISSLESEDFADYY |
| | | | | | CLQRNAYPLTFGAGTKLELR |
| 24 | AT-20.19 | protein | Heavy | Variable | QVQLQQSGRDLVKPGASVKIS |
| | | | | | CKASGYTFTDYHINWVQQRP |
| | | | | | GQGLEWIGWIFPGSGSTYYNE |
| | | | | | KFKGKATLNVDKSSSTAYMLL |
| | | | | | SSLTSEDSAVYFCARGKSGYPF |
| | | | | | YFDYWGQGTTLTVSS |
| 25 | AT-20.19c | protein | Light | CDR L1 | RASQGIRGNLD |
| 26 | AT-20.19c | protein | Light | CDR L2 | STSNLNS |
| 27 | AT-20.19c | protein | Light | CDR L3 | LQRNAYPLT |
| 28 | AT-20.19c | protein | Heavy | CDR H1 | DYHIN |
| 29 | AT-20.19c | protein | Heavy | CDR H2 | WIFPGSGSTYYNEKFKG |
| 30 | AT-20.19c | protein | Heavy | CDR H3 | GKSGYPFYFDY |
| 31 | AT-20.19c | protein | Light | Variable | DIQMIQSPSSMFASLGDRVSLS |
| | | | | | CRASQGIRGNLDWYQQKPGG |
| | | | | | TIKLLIYSTSNLNSGVPSRFSGS |
| | | | | | GSGSDYSLTISSLESEDFADYY |
| | | | | | CLQRNAYPLTFGAGTKLELR |
| 32 | AT-20.19c | protein | Heavy | Variable | QVQLQQSGRDLVKPGASVKIS |
| | | | | | CKASGYTFTDYHINWVQQRP |
| | | | | | GQGLEWIGWIFPGSGSTYYNE |
| | | | | | KFKGKATLNVDKSSSTAYMLL |
| | | | | | SSLTSEDSAVYFCARGKSGYPF |
| | | | | | YFDYWGQGTTLTVSS |
| 33 | AT-20.19h | protein | Light | CDR L1 | RASQGIRGNLD |
| 34 | AT-20.19h | protein | Light | CDR L2 | STSNLNS |
| 35 | AT-20.19h | protein | Light | CDR L3 | LQRNAYPLT |
| 36 | AT-20.19h | protein | Heavy | CDR H1 | DYHIN |
| 37 | AT-20.19h | protein | Heavy | CDR H2 | WIFPGSGSTYYNEKFKG |
| 38 | AT-20.19h | protein | Heavy | CDR H3 | GKSGYPFYFDY |
| 39 | AT-20.19h | protein | Light | Variable | DIQMTQSPSSLSASVGDRVTIT |
| | | | | | CRASQGIRGNLDWYQQKPGK |
| | | | | | APKLLIYSTSNLNSGVPSRFSG |
| | | | | | SGSGSEYTLTISSLQPEDFATYY |
| | | | | | CLQRNAYPLTFGGGTKVEIK |
| 40 | AT-20.19h | protein | Heavy | Variable | QVQLVQSGAEVKKPGASVKV |
| | | | | | SCKASGYTFTDYHINWVRQAT |
| | | | | | GQGLEWIGWIFPGSGSTYYNE |
| | | | | | KFKGRATLTVNKSISTAYMELS |
| | | | | | SLRSEDTAVYYCARGKSGYPF |
| | | | | | YFDYWGQGTLVTVSS |
| 41 | AT-20.25 | protein | Light | CDR L1 | KASENVGTFVS |
| 42 | AT-20.25 | protein | Light | CDR L2 | GASNRYT |
| 43 | AT-20.25 | protein | Light | CDR L3 | GQSYTYPYT |
| 44 | AT-20.25 | protein | Heavy | CDR H1 | SYWMH |
| 45 | AT-20.25 | protein | Heavy | CDR H2 | VIHPNSGSINYNEKFKS |
| 46 | AT-20.25 | protein | Heavy | CDR H3 | GNSLYYALDY |
| 47 | AT-20.25 | protein | Light | Variable | NIVMTQSPKSMSMSVGERVTL |
| | | | | | SCKASENVGTFVSWYQQKPE |
| | | | | | QSPKLLIFGASNRYTGVPDRFT |
| | | | | | GSGSATDFTLIISSVQAEDLAD |
| | | | | | YHCGQSYTYPYTFGGGTKLEIK |
| 48 | AT-20.25 | protein | Heavy | Variable | QVQLQQPGAELVKPGASVKLS |
| | | | | | CKASGYTFTSYWMHWVKQRP |
| | | | | | GQGLEWLGVIHPNSGSINYNE |
| | | | | | KFKSKATLTVDKSSSTAYMQLS |
| | | | | | SLTSEDSAVYYCAGGNSLYYA |
| | | | | | LDYWGQGTSVTVSS |
| 49 | AT-20.25h | protein | Light | CDR L1 | KASENVGTFVS |
| 50 | AT-20.25h | protein | Light | CDR L2 | GASNRYT |
| 51 | AT-20.25h | protein | Light | CDR L3 | GQSYTYPYT |
| 52 | AT-20.25h | protein | Heavy | CDR H1 | SYWMH |
| 53 | AT-20.25h | protein | Heavy | CDR H2 | VIHPNSGSINYNEKFKS |
| 54 | AT-20.25h | protein | Heavy | CDR H3 | GNSLYYALDY |
| 55 | AT-20.25h | protein | Light | Variable | EIVMTQSPATLSLSPGERVTLSC |
| | | | | | KASENVGTFVSWYQQKPGQA |
| | | | | | PRLLIFGASNRYTGIPARFSGSG |
| | | | | | SATDFTLTISSLQPEDFAVYYCG |
| | | | | | QSYTYPYTFGQGTKLEIK |
| 56 | AT-20.25h | protein | Heavy | Variable | QVQLVQSGAEVKKPGASVKV |
| | | | | | SCKASGYTFTSYWMHWVRQA |
| | | | | | PGQGLEWLGVIHPNSGSINYN |
| | | | | | EKFKSRATLTVDKSTSTAYMEL |
| | | | | | SSLRSEDTAVYYCAGGNSLYY |
| | | | | | ALDYWGQGTLVTVSS |
| 57 | AT-20.25hvA | protein | Light | CDR L1 | KASENVGTFVS |
| 58 | AT-20.25hvA | protein | Light | CDR L2 | GASNRYT |
| 59 | AT-20.25hvA | protein | Light | CDR L3 | GQSYTYPYT |
| 60 | AT-20.25hvA | protein | Heavy | CDR H1 | SYWMH |
| 61 | AT-20.25hvA | protein | Heavy | CDR H2 | VIHPNSGSINYNEKFKS |
| 62 | AT-20.25hvA | protein | Heavy | CDR H3 | GNSLYYALDY |
| 63 | AT-20.25hvA | protein | Light | Variable | EIVMTQSPATLSLSPGERVTLSC |
| | | | | | KASENVGTFVSWYQQKPGQA |
| | | | | | PRLLIFGASNRYTGIPARFSGSG |
| | | | | | SGTDFTLTISSLQPEDFAVYYC |
| | | | | | GQSYTYPYTFGQGTKLEIK |
| 64 | AT-20.25hvA | protein | Heavy | Variable | QVQLVQSGAEVKKPGASVKV |
| | | | | | SCKASGYTFTSYWMHWVRQA |
| | | | | | PGQGLEWLGVIHPNSGSINYN |
| | | | | | EKFKSRATLTVDKSTSTAYMEL |
| | | | | | SSLRSEDTAVYYCARGNSLYY |
| | | | | | ALDYWGQGTLVTVSS |
| 65 | AT-20.25hvG | protein | Light | CDR L1 | KASENVGTFVS |
| 66 | AT-20.25hvG | protein | Light | CDR L2 | GASNRYT |
| 67 | AT-20.25hvG | protein | Light | CDR L3 | QQSYTYPYT |
| 68 | AT-20.25hvG | protein | Heavy | CDR H1 | SYWMH |
| 69 | AT-20.25hvG | protein | Heavy | CDR H2 | VIHPNSGSINYNEKFKS |
| 70 | AT-20.25hvG | protein | Heavy | CDR H3 | GNSLYYALDY |
| 71 | AT-20.25hvG | protein | Light | Variable | EIVMTQSPATLSLSPGERVTLSC |
| | | | | | KASENVGTFVSWYQQKPGQA |
| | | | | | PRLLIFGASNRYTGIPARFSGSG |
| | | | | | SATDFTLTISSLQPEDFAVYYCQ |
| | | | | | QSYTYPYTFGQGTKLEIK |
| 72 | AT-20.25hvG | protein | Heavy | Variable | QVQLVQSGAEVKKPGASVKV |
| | | | | | SCKASGYTFTSYWMHWVRQA |
| | | | | | PGQGLEWLGVIHPNSGSINYN |
| | | | | | EKFKSRATLTVDKSTSTAYMEL |
| | | | | | SSLRSEDTAVYYCARGNSLYY |
| | | | | | ALDYWGQGTLVTVSS |
| 73 | AT-20.25hvAG | protein | Light | CDR L1 | KASENVGTFVS |
| 74 | AT-20.25hvAG | protein | Light | CDR L2 | GASNRYT |
| 75 | AT-20.25hvAG | protein | Light | CDR L3 | QQSYTYPYT |
| 76 | AT-20.25hvAG | protein | Heavy | CDR H1 | SYWMH |
| 77 | AT-20.25hvAG | protein | Heavy | CDR H2 | VIHPNSGSINYNEKFKS |
| 78 | AT-20.25hvAG | protein | Heavy | CDR H3 | GNSLYYALDY |
| 79 | AT-20.25hvAG | protein | Light | Variable | EIVMTQSPATLSLSPGERVTLSC |
| | | | | | KASENVGTFVSWYQQKPGQA |
| | | | | | PRLLIFGASNRYTGIPARFSGSG |
| | | | | | SGTDFTLTISSLQPEDFAVYYC |
| | | | | | QQSYTYPYTFGQGTKLEIK |
| 80 | AT-20.25hvAG | protein | Heavy | Variable | QVQLVQSGAEVKKPGASVKV |
| | | | | | SCKASGYTFTSYWMHWVRQA |
| | | | | | PGQGLEWLGVIHPNSGSINYN |
| | | | | | EKFKSRATLTVDKSTSTAYMEL |
| | | | | | SSLRSEDTAVYYCARGNSLYY |
| | | | | | ALDYWGQGTLVTVSS |
| 81 | AT-20.10 | DNA | Light | Variable | GACATAGTCCTGACACAGAG |
| | | | | | CCCTGCGAGCCTGGCCGTGA |
| | | | | | GCCTGGGGCAGAGAGCCACC |
| | | | | | ATCAGCTGCAGAGCTAGCGA |
| | | | | | GAGCGTGGACAACTACGGCA |
| | | | | | TCAGCTTCATGAACTGGTTTC |
| | | | | | AGCAGAAGCCCGGGCAGCCC |
| | | | | | CCCAAGCTGCTTATCTATGCA |
| | | | | | GCTAGCAACCAAGGCAGCGG |
| | | | | | CGTTCCCGCTAGATTCAGCGG |
| | | | | | CAGCGGCAGCGGCACCGACT |
| | | | | | TCAGCCTGAACATCCACCCC |
| | | | | | ATGGAGGAGGACGACACCGC |
| | | | | | CATGTACTTCTGTCAGCAGAG |
| | | | | | CAAGGAGATCCCCCTGACCT |
| | | | | | TCGGCGCCGGCACCAAGCTG |
| | | | | | GAGCTGAAG |
| 82 | AT-20.10 | DNA | Heavy | Variable | CAAGTGCAGCTGCAGCAAAG |
| | | | | | CGGTCCTGACCTGGTGAGAC |
| | | | | | CCGGTACAAGCGTGAAGATC |
| | | | | | AGCTGCAAGGGCAGCGGCTA |
| | | | | | CACCTTCCTGACACAGTGGAT |
| | | | | | GAACTGGGTGAAGCAGAGAC |
| | | | | | CCGGCCAAGGCCTGGAGTGG |
| | | | | | ATCGGGCAGATCTTCCCCGCT |
| | | | | | AGCGGCAGCACCTACTACAA |
| | | | | | CGAGATGTTCAAGGACAAGG |
| | | | | | CCACCCTGACCGTGGACACA |
| | | | | | AGCAGCAGCACCGCCTACAT |
| | | | | | GCAGCTGAGCAGCCTGACAA |
| | | | | | GCGAGGACACCGCCGTGTAC |
| | | | | | TTCTGCGCTAGAAGAGGCGG |
| | | | | | CAGCGGCATGGACTACTGGG |
| | | | | | GCCAAGGCACAAGCGTGACC |
| | | | | | GTGAGCAGC |
| 83 | AT-20.19 | DNA | Light | Variable | GACATTCAGATGATTCAGAGC |
| | | | | | CCTAGCAGCATGTTCGCTAGC |
| | | | | | CTGGGCGACAGAGTGAGCCT |
| | | | | | GAGCTGCAGAGCTAGCCAAG |
| | | | | | GCATCAGAGGCAACCTGGAC |
| | | | | | TGGTATCAGCAGAAGCCCGG |
| | | | | | CGGCACCATCAAGCTGCTGAT |
| | | | | | CTACAGCACAAGCAACCTGA |
| | | | | | ACAGCGGCGTGCCTAGCAGA |
| | | | | | TTCAGCGGCAGCGGCAGCGG |
| | | | | | CAGCGACTACAGCCTGACCA |
| | | | | | TCAGCAGCCTGGAGAGCGAG |
| | | | | | GACTTCGCCGACTACTACTGC |
| | | | | | CTGCAGAGAAACGCCTACCC |
| | | | | | CCTGACCTTCGGCGCCGGCA |
| | | | | | CCAAGCTGGAGCTG |
| 84 | AT-20.19 | DNA | Heavy | Variable | CAAGTGCAGCTGCAGCAGAG |
| | | | | | CGGCAGAGACCTGGTGAAGC |
| | | | | | CCGGCGCTAGCGTGAAGATC |
| | | | | | AGCTGCAAGGCTAGCGGCTA |
| | | | | | CACCTTCACCGACTACCACAT |
| | | | | | CAACTGGGTGCAGCAGAGAC |
| | | | | | CCGGCCAAGGCCTGGAGTGG |
| | | | | | ATCGGCTGGATCTTCCCCGGC |
| | | | | | AGCGGCAGCACCTACTACAA |
| | | | | | CGAGAAGTTCAAGGGCAAGG |
| | | | | | CCACCCTGAACGTGGACAAG |
| | | | | | AGCAGCAGCACCGCCTACAT |
| | | | | | GCTGCTGAGCAGCCTGACAA |
| | | | | | GCGAGGACAGCGCCGTGTAC |
| | | | | | TTCTGCGCTAGAGGCAAGAG |
| | | | | | CGGCTACCCCTTCTACTTCGA |
| | | | | | CTACTGGGGCCAAGGCACCA |
| | | | | | CCCTGACCGTGAGCAGC |
| 85 | AT-20.25 | DNA | Light | Variable | AACATAGTTATGACTCAGTCT |
| | | | | | CCGAAATCCATGTCCATGTCT |
| | | | | | GTTGGGGAAAGAGTCACGCT |
| | | | | | CAGTTGTAAAGCCAGTGAGA |
| | | | | | ACGTGGGAACTTTTGTGTCCT |
| | | | | | GGTACCAGCAAAAGCCCGAA |
| | | | | | CAGTCTCCGAAACTTTTGATC |
| | | | | | TTTGGGGCCAGCAATAGATAC |
| | | | | | ACCGGGGTCCCCGATCGTTTC |
| | | | | | ACGGGTTCTGGTTCTGCCAC |
| | | | | | AGACTTTACTCTCATCATTAG |
| | | | | | TTCTGTACAAGCTGAAGATTT |
| | | | | | GGCCGACTATCACTGTGGTCA |
| | | | | | GTCTTACACATATCCCTACAC |
| | | | | | TTTCGGTGGCGGAACAAAAC |
| | | | | | TGGAAATTAAA |
| 86 | AT-20.25 | DNA | Heavy | Variable | CAAGTTCAACTACAACAACC |
| | | | | | CGGGGCTGAACTGGTAAAAC |
| | | | | | CTGGAGCTTCTGTAAAACTGT |
| | | | | | CCTGTAAGGCAAGTGGTTATA |
| | | | | | CATTTACTTCATATTGGATGCA |
| | | | | | TTGGGTGAAACAACGTCCAG |
| | | | | | GTCAAGGACTGGAATGGCTT |
| | | | | | GGTGTGATTCACCCCAATTCT |
| | | | | | GGATCCATAAATTACAACGAA |
| | | | | | AAGTTTAAATCTAAAGCTACT |
| | | | | | CTCACGGTTGACAAATCCTCA |
| | | | | | AGTACGGCATATATGCAACTC |
| | | | | | TCTTCTTTGACTTCCGAGGAC |
| | | | | | AGCGCAGTCTACTACTGTGCC |
| | | | | | GGCGGGAATTCTCTGTACTAT |
| | | | | | GCCTTGGA |
| | | | | | CTATTGGGGACAAGGCACAA |
| | | | | | GCGTGACCGTGAGCTCT |
| 87 | AT-20.25h | DNA | Light | Variable | GAGATTGTAATGACCCAAAGT |
| | | | | | CCAGCAACCTTGTCTTTGTCA |
| | | | | | CCTGGGGAGCGGGTAACTTT |
| | | | | | GTCATGTAAGGCTTCAGAGA |
| | | | | | ATGTCGGAACCTTCGTAAGCT |
| | | | | | GGTACCAACAGAAACCCGGA |
| | | | | | CAAGCTCCACGCCTGTTGATC |
| | | | | | TTCGGCGCTTCCAATCGGTAC |
| | | | | | ACAGGGATTCCTGCACGCTTC |
| | | | | | TCAGGGTCCGGTTCCGCTACC |
| | | | | | GATTTTACTCTAACTATATCTA |
| | | | | | GCCTACAGCCTGAAGATTTTG |
| | | | | | CCGTCTACTACTGTGGGCAAT |
| | | | | | CATATACATACCCATATACGTT |
| | | | | | CGGCCAAGGCACCAAACTTG |
| | | | | | AGATCAAA |
| 88 | AT-20.25h | DNA | Heavy | Variable | CAAGTACAACTAGTCCAATCC |
| | | | | | GGCGCAGAAGTGAAGAAACC |
| | | | | | CGGCGCTTCCGTCAAAGTGA |
| | | | | | GCTGTAAGGCTTCAGGGTAC |
| | | | | | ACATTCACCTCATATTGGATG |
| | | | | | CACTGGGTTAGGCAGGCTCC |
| | | | | | TGGACAGGGCCTCGAGTGGC |
| | | | | | TTGGAGTGATACACCCAAACT |
| | | | | | CAGGCTCCATCAACTATAACG |
| | | | | | AAAAGTTTAAATCCCGTGCTA |
| | | | | | CTCTAACAGTGGACAAGAGT |
| | | | | | ACAAGCACTGCTTACATGGA |
| | | | | | ATTGTCAAGTCTGCGAAGCG |
| | | | | | AAGACACTGCTGTTTATTACT |
| | | | | | GTGCAGGTGGGAATTCCCTG |
| | | | | | TATTACGCTCTTGACTATTGG |
| | | | | | GGCCAGGGTACACTGGTCAC |
| | | | | | CGTCTCTAGC |

### INFORMAL SEQUENCE LISTING

AT-20 Assets (e.g., anti-CDH17 antibodies described herein):
AT-20.10 Light chain CDR1 amino acid sequence (SEQ ID NO: 1):
IASQSVDNYGISFMN
AT-20.10 Light chain CDR2 amino acid sequence (SEQ ID NO: 2):
AASNQGS
AT-20.10 Light chain CDR3 amino acid sequence (SEQ ID NO: 3):
QQSKEIPLT
AT-20.10 Heavy chain CDRI amino acid sequence (SEQ ID NO: 4):
TQWMN
AT-20.10 Heavy chain CDR2 amino acid sequence (SEQ ID NO: 5):
QIFPASGSTYYNEMFKD
AT-20.10 Heavy chain CDR3 amino acid sequence (SEQ ID NO: 6):
RGGSGMDY
AT-20.10 Light chain variable region amino acid sequence (SEQ ID NO: 7):
DIVLTQSPASLAVSLRHRATISCIASQSVDNYGISFMNWFQHKPGQPPKLLIYAASNQGSGVPA
RFSGSGSGTDFSLNIHPMEEDDTAMYFCQQSKEIPLTFGAGTKLELK
AT-20.10 Heavy chain variable region amino acid sequence (SEQ ID NO: 8):
QVQLQQSGPDLVRPGTSVKISCKGSGYTFLTQWMNWVKQRPGQGLEWIGQIFPASGSTYYNE
MFKDKATLTVDTSSSTAYMQLSSLTSEDTAVYFCARRGGSGMDYWGQGTSVTVSS
AT-20.10c Light chain CDR1 amino acid sequence (SEQ ID NO: 9):
IASQSVDNYGISFMN
AT-20.10c Light chain CDR2 amino acid sequence (SEQ ID NO: 10):
AASNQGS
AT-20.10c Light chain CDR3 amino acid sequence (SEQ ID NO: 11):
QQSKEIPLT
AT-20.10c Heavy chain CDR1 amino acid sequence (SEQ ID NO: 12):
TQWMN
AT-20.10c Heavy chain CDR2 amino acid sequence (SEQ ID NO: 13):
QIFPASGSTYYNEMFKD
AT-20.10c Heavy chain CDR3 amino acid sequence (SEQ ID NO: 14):
RGGSGMDY
AT-20.10c Light chain variable region amino acid sequence (SEQ ID NO: 15):
DIVLTQSPASLAVSLRHRATISCIASQSVDNYGISFMNWFQHKPGQPPKLLIYAASNQGSGVPA
RFSGSGSGTDFSLNIHPMEEDDTAMYFCQQSKEIPLTFGAGTKLELK
AT-20.10c Heavy chain variable region amino acid sequence (SEQ ID NO: 16):
QVQLQQSGPDLVRPGTSVKISCKGSGYTFLTQWMNWVKQRPGQGLEWIGQIFPASGSTYYNE
MFKDKATLTVDTSSSTAYMQLSSLTSEDTAVYFCARRGGSGMDYWGQGTSVTVSS
AT-20.19 Light chain CDR1 amino acid sequence (SEQ ID NO: 17):
RASQGIRGNLD
AT-20.19 Light chain CDR2 amino acid sequence (SEQ ID NO: 18):
STSNLNS
AT-20.19 Light chain CDR3 amino acid sequence (SEQ ID NO: 19):
LQRNAYPLT
AT-20.19 Heavy chain CDRI amino acid sequence (SEQ ID NO: 20):
DYHIN
AT-20.19 Heavy chain CDR2 amino acid sequence (SEQ ID NO: 21):
WIFPGSGSTYYNEKFKG
AT-20.19 Heavy chain CDR3 amino acid sequence (SEQ ID NO: 22):
GKSGYPFYFDY
AT-20.19 Light chain variable region amino acid sequence (SEQ ID NO: 23):
DIQMIQSPSSMFASLGDRVSLSCRASQGIRGNLDWYQQKPGGTIKLLIYSTSNLNSGVPSRFSG
SGSGSDYSLTISSLESEDFADYYCLQRNAYPLTFGAGTKLELR
AT-20.19 Heavy chain variable region amino acid sequence (SEQ ID NO: 24):
QVQLQQSGRDLVKPGASVKISCKASGYTFTDYHINWVQQRPGQGLEWIGWIFPGSGSTYYNE
KFKGKATLNVDKSSSTAYMLLSSLTSEDSAVYFCARGKSGYPFYFDYWGQGTTLTVSS
AT-20.19c Light chain CDRI amino acid sequence (SEQ ID NO: 25):
RASQGIRGNLD
AT-20.19c Light chain CDR2 amino acid sequence (SEQ ID NO: 26):
STSNLNS
AT-20.19c Light chain CDR3 amino acid sequence (SEQ ID NO: 27):
LQRNAYPLT
AT-20.19c Heavy chain CDR1 amino acid sequence (SEQ ID NO: 28):
DYHIN
AT-20.19c Heavy chain CDR2 amino acid sequence (SEQ ID NO: 29):
WIFPGSGSTYYNEKFKG
AT-20.19c Heavy chain CDR3 amino acid sequence (SEQ ID NO: 30):
GKSGYPFYFDY
AT-20.19c Light chain variable region amino acid sequence (SEQ ID NO: 31):
DIQMIQSPSSMFASLGDRVSLSCRASQGIRGNLDWYQQKPGGTIKLLIYSTSNLNSGVPSRFSG
SGSGSDYSLTISSLESEDFADYYCLQRNAYPLTFGAGTKLELR
AT-20.19c Heavy chain variable region amino acid sequence (SEQ ID NO: 32):
QVQLQQSGRDLVKPGASVKISCKASGYTFTDYHINWVQQRPGQGLEWIGWIFPGSGSTYYNE
KFKGKATLNVDKSSSTAYMLLSSLTSEDSAVYFCARGKSGYPFYFDYWGQGTTLTVSS
AT-20.19h Light chain CDR1 amino acid sequence (SEQ ID NO: 33):
RASQGIRGNLD
AT-20.19h Light chain CDR2 amino acid sequence (SEQ ID NO: 34):
STSNLNS
AT-20.19h Light chain CDR3 amino acid sequence (SEQ ID NO: 35):
LQRNAYPLT
AT-20.19h Heavy chain CDR1 amino acid sequence (SEQ ID NO: 36):
DYHIN
AT-20.19h Heavy chain CDR2 amino acid sequence (SEQ ID NO: 37):
WIFPGSGSTYYNEKFKG
AT-20.19h Heavy chain CDR3 amino acid sequence (SEQ ID NO: 38):
GKSGYPFYFDY
AT-20.19h Light chain variable region amino acid sequence (SEQ ID NO: 39):
DIQMTQSPSSLSASVGDRVTITCRASQGIRGNLDWYQQKPGKAPKLLIYSTSNLNSGVPSRFS
GSGSGSEYTLTISSLQPEDFATYYCLQRNAYPLTFGGGTKVEIK
AT-20.19h Heavy chain variable region amino acid sequence (SEQ ID NO: 40):
QVQLVQSGAEVKKPGASVKVSCKASGYTFTDYHINWVRQATGQGLEWIGWIFPGSGSTYYN
EKFKGRATLTVNKSISTAYMELSSLRSEDTAVYYCARGKSGYPFYFDYWGQGTLVTVSS
AT-20.25 Light chain CDR1 amino acid sequence (SEQ ID NO: 41):
KASENVGTFVS
AT-20.25 Light chain CDR2 amino acid sequence (SEQ ID NO: 42):
GASNRYT
AT-20.25 Light chain CDR3 amino acid sequence (SEQ ID NO: 43):
GQSYTYPYT
AT-20.25 Heavy chain CDR1 amino acid sequence (SEQ ID NO: 44):
SYWMH
AT-20.25 Heavy chain CDR2 amino acid sequence (SEQ ID NO: 45):
VIHPNSGSINYNEKFKS
AT-20.25 Heavy chain CDR3 amino acid sequence (SEQ ID NO: 46):
GNSLYYALDY
AT-20.25 Light chain variable region amino acid sequence (SEQ ID NO: 47):
NIVMTQSPKSMSMSVGERVTLSCKASENVGTFVSWYQQKPEQSPKLLIFGASNRYTGVPDRF
TGSGSATDFTLIISSVQAEDLADYHCGQSYTYPYTFGGGTKLEIK
AT-20.25 Heavy chain variable region amino acid sequence (SEQ ID NO: 48):
QVQLQQPGAELVKPGASVKLSCKASGYTFTSYWMHWVKQRPGQGLEWLGVIHPNSGSINY
NEKFKSKATLTVDKSSSTAYMQLSSLTSEDSAVYYCAGGNSLYYALDYWGQGTSVTVSS
AT-20.25h Light chain CDR1 amino acid sequence (SEQ ID NO: 49):
KASENVGTFVS
AT-20.25h Light chain CDR2 amino acid sequence (SEQ ID NO: 50):
GASNRYT
AT-20.25h Light chain CDR3 amino acid sequence (SEQ ID NO: 51):
GQSYTYPYT
AT-20.25h Heavy chain CDR1 amino acid sequence (SEQ ID NO: 52):
SYWMH
AT-20.25h Heavy chain CDR2 amino acid sequence (SEQ ID NO: 53):
VIHPNSGSINYNEKFKS
AT-20.25h Heavy chain CDR3 amino acid sequence (SEQ ID NO: 54):
GNSLYYALDY
AT-20.25h Light chain variable region amino acid sequence (SEQ ID NO: 55):
EIVMTQSPATLSLSPGERVTLSCKASENVGTFVSWYQQKPGQAPRLLIFGASNRYTGIPARFSG
SGSATDFTLTISSLQPEDFAVYYCGQSYTYPYTFGQGTKLEIK
AT-20.25h Heavy chain variable region amino acid sequence (SEQ ID NO: 56):
QVQLVQSGAEVKKPGASVKVSCKASGYTFTSYWMHWVRQAPGQGLEWLGVIHPNSGSINY
NEKFKSRATLTVDKSTSTAYMELSSLRSEDTAVYYCAGGNSLYYALDYWGQGTLVTVSS
AT-20.25hvA Light chain CDR1 amino acid sequence (SEQ ID NO: 57):
KASENVGTFVS
AT-20.25hvA Light chain CDR2 amino acid sequence (SEQ ID NO: 58):
GASNRYT
AT-20.25hvA Light chain CDR3 amino acid sequence (SEQ ID NO: 59):
GQSYTYPYT
AT-20.25hvA Heavy chain CDR1 amino acid sequence (SEQ ID NO: 60):
SYWMH
AT-20.25hvA Heavy chain CDR2 amino acid sequence (SEQ ID NO: 61):
VIHPNSGSINYNEKFKS
AT-20.25hvA Heavy chain CDR3 amino acid sequence (SEQ ID NO: 62):
GNSLYYALDY
AT-20.25hvA Light chain variable region amino acid sequence (SEQ ID NO: 63):
EIVMTQSPATLSLSPGERVTLSCKASENVGTFVSWYQQKPGQAPRLLIFGASNRYTGIPARFSG
SGSGTDFTLTISSLQPEDFAVYYCGQSYTYPYTFGQGTKLEIK
AT-20.25hvA Heavy chain variable region amino acid sequence (SEQ ID NO: 64):
QVQLVQSGAEVKKPGASVKVSCKASGYTFTSYWMHWVRQAPGQGLEWLGVIHPNSGSINY
NEKFKSRATLTVDKSTSTAYMELSSLRSEDTAVYYCARGNSLYYALDYWGQGTLVTVSS
AT-20.25hvG Light chain CDR1 amino acid sequence (SEQ ID NO: 65):
KASENVGTFVS
AT-20.25hvG Light chain CDR2 amino acid sequence (SEQ ID NO: 66):
GASNRYT
AT-20.25hvG Light chain CDR3 amino acid sequence (SEQ ID NO: 67):
QQSYTYPYT
AT-20.25hvG Heavy chain CDR1 amino acid sequence (SEQ ID NO: 68):
SYWMH
AT-20.25hvG Heavy chain CDR2 amino acid sequence (SEQ ID NO: 69):
VIHPNSGSINYNEKFKS
AT-20.25hvG Heavy chain CDR3 amino acid sequence (SEQ ID NO: 70):
GNSLYYALDY
AT-20.25hvG Light chain variable region amino acid sequence (SEQ ID NO: 71):
EIVMTQSPATLSLSPGERVTLSCKASENVGTFVSWYQQKPGQAPRLLIFGASNRYTGIPARFSG
SGSATDFTLTISSLQPEDFAVYYCQQSYTYPYTFGQGTKLEIK
AT-20.25hvG Heavy chain variable region amino acid sequence (SEQ ID NO: 72):
QVQLVQSGAEVKKPGASVKVSCKASGYTFTSYWMHWVRQAPGQGLEWLGVIHPNSGSINY
NEKFKSRATLTVDKSTSTAYMELSSLRSEDTAVYYCARGNSLYYALDYWGQGTLVTVSS
AT-20.25hvAG Light chain CDRI amino acid sequence (SEQ ID NO: 73):
KASENVGTFVS
AT-20.25hvAG Light chain CDR2 amino acid sequence (SEQ ID NO: 74):
GASNRYT
AT-20.25hvAG Light chain CDR3 amino acid sequence (SEQ ID NO: 75):
QQSYTYPYT
AT-20.25hvAG Heavy chain CDR1 amino acid sequence (SEQ ID NO: 76):
SYWMH
AT-20.25hvAG Heavy chain CDR2 amino acid sequence (SEQ ID NO: 77):
VIHPNSGSINYNEKFKS
AT-20.25hvAG Heavy chain CDR3 amino acid sequence (SEQ ID NO: 78):
GNSLYYALDY
AT-20.25hvAG Light chain variable region amino acid sequence (SEQ ID NO: 79):
EIVMTQSPATLSLSPGERVTLSCKASENVGTFVSWYQQKPGQAPRLLIFGASNRYTGIPARFSG
SGSGTDFTLTISSLQPEDFAVYYCQQSYTYPYTFGQGTKLEIK
AT-20.25hvAG Heavy chain variable region amino acid sequence (SEQ ID NO: 80):
QVQLVQSGAEVKKPGASVKVSCKASGYTFTSYWMHWVRQAPGQGLEWLGVIHPNSGSINY
NEKFKSRATLTVDKSTSTAYMELSSLRSEDTAVYYCARGNSLYYALDYWGQGTLVTVSS
AT-20.10 Light chain variable region DNA sequence (SEQ ID NO: 81):
GACATAGTCCTGACACAGAGCCCTGCGAGCCTGGCCGTGAGCCTGGGGCAGAGAGCCAC
CATCAGCTGCAGAGCTAGCGAGAGCGTGGACAACTACGGCATCAGCTTCATGAACTGGTT
TCAGCAGAAGCCCGGGCAGCCCCCCAAGCTGCTTATCTATGCAGCTAGCAACCAAGGCAG
CGGCGTTCCCGCTAGATTCAGCGGCAGCGGCAGCGGCACCGACTTCAGCCTGAACATCCA
CCCCATGGAGGAGGACGACACCGCCATGTACTTCTGTCAGCAGAGCAAGGAGATCCCCCT
GACCTTCGGCGCCGGCACCAAGCTGGAGCTGAAG
AT-20.10 Heavy chain variable region DNA sequence (SEQ ID NO: 82):
CAAGTGCAGCTGCAGCAAAGCGGTCCTGACCTGGTGAGACCCGGTACAAGCGTGAAGAT
CAGCTGCAAGGGCAGCGGCTACACCTTCCTGACACAGTGGATGAACTGGGTGAAGCAGA
GACCCGGCCAAGGCCTGGAGTGGATCGGGCAGATCTTCCCCGCTAGCGGCAGCACCTACT
ACAACGAGATGTTCAAGGACAAGGCCACCCTGACCGTGGACACAAGCAGCAGCACCGCC
TACATGCAGCTGAGCAGCCTGACAAGCGAGGACACCGCCGTGTACTTCTGCGCTAGAAGA
GGCGGCAGCGGCATGGACTACTGGGGCCAAGGCACAAGCGTGACCGTGAGCAGC
AT-20.19 Light chain variable region DNA sequence (SEQ ID NO: 83):
GACATTCAGATGATTCAGAGCCCTAGCAGCATGTTCGCTAGCCTGGGCGACAGAGTGAGC
CTGAGCTGCAGAGCTAGCCAAGGCATCAGAGGCAACCTGGACTGGTATCAGCAGAAGCC
CGGCGGCACCATCAAGCTGCTGATCTACAGCACAAGCAACCTGAACAGCGGCGTGCCTAG
CAGATTCAGCGGCAGCGGCAGCGGCAGCGACTACAGCCTGACCATCAGCAGCCTGGAGA
GCGAGGACTTCGCCGACTACTACTGCCTGCAGAGAAACGCCTACCCCCTGACCTTCGGCG
CCGGCACCAAGCTGGAGCTG
AT-20.19 Heavy chain variable region DNA sequence (SEQ ID NO: 84):
CAAGTGCAGCTGCAGCAGAGCGGCAGAGACCTGGTGAAGCCCGGCGCTAGCGTGAAGAT
CAGCTGCAAGGCTAGCGGCTACACCTTCACCGACTACCACATCAACTGGGTGCAGCAGAG
ACCCGGCCAAGGCCTGGAGTGGATCGGCTGGATCTTCCCCGGCAGCGGCAGCACCTACTA
CAACGAGAAGTTCAAGGGCAAGGCCACCCTGAACGTGGACAAGAGCAGCAGCACCGCC
TACATGCTGCTGAGCAGCCTGACAAGCGAGGACAGCGCCGTGTACTTCTGCGCTAGAGGC
AAGAGCGGCTACCCCTTCTACTTCGACTACTGGGGCCAAGGCACCACCCTGACCGTGAGC
AGC
AT-20.25 Light chain variable region DNA sequence (SEQ ID NO: 85):
AACATAGTTATGACTCAGTCTCCGAAATCCATGTCCATGTCTGTTGGGGAAAGAGTCACGC
TCAGTTGTAAAGCCAGTGAGAACGTGGGAACTTTTGTGTCCTGGTACCAGCAAAAGCCCG
AACAGTCTCCGAAACTTTTGATCTTTGGGGCCAGCAATAGATACACCGGGGTCCCCGATCG
TTTCACGGGTTCTGGTTCTGCCACAGACTTTACTCTCATCATTAGTTCTGTACAAGCTGAA
GATTTGGCCGACTATCACTGTGGTCAGTCTTACACATATCCCTACACTTTCGGTGGCGGAA
CAAAACTGGAAATTAAA
AT-20.25 Heavy chain variable region DNA sequence (SEQ ID NO: 86):
CAAGTTCAACTACAACAACCCGGGGCTGAACTGGTAAAACCTGGAGCTTCTGTAAAACTG
TCCTGTAAGGCAAGTGGTTATACATTTACTTCATATTGGATGCATTGGGTGAAACAACGTCC
AGGTCAAGGACTGGAATGGCTTGGTGTGATTCACCCCAATTCTGGATCCATAAATTACAAC
GAAAAGTTTAAATCTAAAGCTACTCTCACGGTTGACAAATCCTCAAGTACGGCATATATGC
AACTCTCTTCTTTGACTTCCGAGGACAGCGCAGTCTACTACTGTGCCGGCGGGAATTCTCT
GTACTATGCCTTGGACTATTGGGGACAAGGCACAAGCGTGACCGTGAGCTCT
AT-20.25h Light chain variable region DNA sequence (SEQ ID NO: 87):
GAGATTGTAATGACCCAAAGTCCAGCAACCTTGTCTTTGTCACCTGGGGAGCGGGTAACT
TTGTCATGTAAGGCTTCAGAGAATGTCGGAACCTTCGTAAGCTGGTACCAACAGAAACCC
GGACAAGCTCCACGCCTGTTGATCTTCGGCGCTTCCAATCGGTACACAGGGATTCCTGCAC
GCTTCTCAGGGTCCGGTTCCGCTACCGATTTTACTCTAACTATATCTAGCCTACAGCCTGAA
GATTTTGCCGTCTACTACTGTGGGCAATCATATACATACCCATATACGTTCGGCCAAGGCAC
CAAACTTGAGATCAAA
AT-20.25h Heavy chain variable region DNA sequence (SEQ ID NO: 88):
CAAGTACAACTAGTCCAATCCGGCGCAGAAGTGAAGAAACCCGGCGCTTCCGTCAAAGT
GAGCTGTAAGGCTTCAGGGTACACATTCACCTCATATTGGATGCACTGGGTTAGGCAGGCT
CCTGGACAGGGCCTCGAGTGGCTTGGAGTGATACACCCAAACTCAGGCTCCATCAACTAT
AACGAAAAGTTTAAATCCCGTGCTACTCTAACAGTGGACAAGAGTACAAGCACTGCTTAC
ATGGAATTGTCAAGTCTGCGAAGCGAAGACACTGCTGTTTATTACTGTGCAGGTGGGAATT
CCCTGTATTACGCTCTTGACTATTGGGGCCAGGGTACACTGGTCACCGTCTCTAGC

### NUMBERED EMBODIMENTS

Below are example embodiments of aspects of the subject matter disclosed herein. It is to be understood that these represent only some aspects of the subject matter of the inventions disclosed in this document and that other embodiments of the subject matter are intentionally disclosed herein.
Embodiment 1. An anti-cadherin-17 (CDH17) antibody comprising a light chain variable domain and a heavy chain variable domain, wherein the light chain variable domain comprises: a CDR L1, a CDR L2, and a CDR L3 as set forth in Table 1; and wherein the heavy chain variable domain comprises: a CDR H1, a CDR H2, and a CDR H3 as set forth in Table 1.
Embodiment 2. The antibody of embodiment 1, wherein the light chain variable domain comprises an amino acid sequence with at least 80%, 85%, 90%, 91%, 92%, 93%, 94%, 95%, 96%, 97%, 98%, 99%, or 100% sequence identity with a light chain variable domain sequence as set forth in Table 1.
Embodiment 3. The antibody of embodiment 1 or embodiment 2, wherein the heavy chain variable domain comprises an amino acid sequence with at least 80%, 85%, 90%, 91%, 92%, 93%, 94%, 95%, 96%, 97%, 98%, 99%, or 100% sequence identity with heavy chain variable domain sequence as set forth in Table 1.
Embodiment 4. The antibody of embodiment 1, wherein the antibody is a humanized antibody.
Embodiment 5. The antibody of any one of embodiments 1-3, wherein the antibody is a chimeric antibody.
Embodiment 6. The antibody of any one of embodiments 1-5, wherein the antibody is an immunoglobulin G (IgG).
Embodiment 7. The antibody of embodiment 6, wherein the antibody is an IgG1.
Embodiment 8. The antibody of any one of embodiments 1-4, wherein the antibody is a Fab′ fragment.
Embodiment The antibody of any one of embodiments 1-3, wherein the antibody is a single chain antibody (scFv).
Embodiment 10. The antibody of any one of embodiments 1-3, wherein the light chain variable domain and said heavy chain variable domain form part of a scFv.
Embodiment 11. The antibody of any one of embodiments 1-10, wherein the antibody is capable of binding cadherin-17 (CDH17).
Embodiment 12. The antibody of any one of embodiments 1-11, wherein the antibody is bound to CDH17.
Embodiment 13. The antibody of embodiment 11 or embodiment 12, wherein the CDH17 forms part of a cell.
Embodiment 14. The antibody of embodiment 13, wherein the cell is a cancer cell.
Embodiment 15. The antibody of embodiment 14, wherein the cancer cell is a gastrointestinal cancer cell, a colorectal cancer cell, a stomach cancer cell, a pancreas cancer cell, a neuroendocrine cancer cell, or an ovarian cancer cell.
Embodiment 16. An anti-cadherin-17 (CDH17) antibody, wherein the anti-CDH17 antibody binds the same epitope as an antibody comprising: a light chain variable region domain comprising a CDR L1, a CDR L2, and a CDR L3 as set forth in Table 1 and a heavy chain variable domain comprising a CDR H1, a CDR H2, and a CDR H3 as set forth in Table 1.
Embodiment 17. The anti-CDH17 antibody of embodiment 16, wherein the light chain variable domain comprises an amino acid sequence with at least 80%, 85%, 90%, 91%, 92%, 93%, 94%, 95%, 96%, 97%, 98%, 99%, or 100% sequence identity with a light chain variable domain sequence as set forth in Table 1
Embodiment 18. The anti-CDH17 antibody of embodiment 16 or embodiment 17, wherein the heavy chain variable domain comprises an amino acid sequence with at least 80%, 85%, 90%, 91%, 92%, 93%, 94%, 95%, 96%, 97%, 98%, 99%, or 100% sequence identity with a heavy chain variable domain sequence as set forth in Table 1.
Embodiment 19. The anti-CDH17 antibody of any one of the preceding embodiments, wherein the antibody is conjugated to a payload.
Embodiment 20. The anti-CDH17 antibody of embodiment 19, wherein the payload is an antineoplastic agent.
Embodiment 21. The anti-CDH17 antibody of embodiment 19 or embodiment 20, wherein the payload is selected from monomethyl auristatin E (MMAE), DX8951, or DXd.
Embodiment 22. The anti-CDH17 antibody of any one of the preceding embodiments, wherein the antibody is conjugated to a linker molecule.
Embodiment 23. The anti-CDH17 antibody of embodiment 22, wherein the linker is selected from MC-Val-Cit-PAB or a tetrapeptide.
Embodiment 24. The anti-CDH17 antibody of embodiment 23, wherein the tetrapeptide is GGFG.
Embodiment 25. A kit comprising: the anti-CDH17 antibody of any one of the preceding embodiments and instructions for use in reducing the viability of a cancer cell or a tumor cell.
Embodiment 26. An isolated nucleic acid encoding the antibody of any one of embodiments 1-24.
Embodiment 27. The isolated nucleic acid of embodiment 26, wherein the antibody comprises a light chain variable domain sequence and a heavy chain variable domain sequence, wherein the light chain variable domain sequence comprises: a CDR L1, a CDR L2, and a CDR L3 as set forth in Table 1; and wherein the heavy chain variable domain sequence comprises: a CDR H1, a CDR H2, and a CDR H3 as set forth in Table 1.
Embodiment 28. The isolated nucleic acid of embodiment 27, wherein the light chain variable domain sequence is encoded by a nucleic acid sequence with at least 80%, 85%, 90%, 91%, 92%, 93%, 94%, 95%, 96%, 97%, 98%, 99%, or 100% sequence identity a light chain variable nucleotide sequence as set forth in Table 1.
Embodiment 29. The isolated nucleic acid of embodiment 27 or embodiment 28, wherein the heavy chain variable domain is encoded by a nucleic acid sequence with at least 80%, 85%, 90%, 91%, 92%, 93%, 94%, 95%, 96%, 97%, 98%, 99%, or 100% sequence identity with a heavy chain variable nucleotide sequence as set forth in Table 1.
Embodiment 30. A cell comprising the isolated nucleic acid of any one of embodiments 26-29.
Embodiment 31. A pharmaceutical composition comprising a therapeutically effective amount of the antibody of any one of embodiments 1-24 or the isolated nucleic acid of any one of embodiments 26-29; and a pharmaceutically acceptable excipient.
Embodiment 32. The pharmaceutical composition of embodiment 31, wherein the antibody is formulated in an aqueous solution at a concentration of 0.2 mg/mL to 2 mg/mL, 0.5 mg/mL to 1.5 mg/mL, 0.75 mg/mL to 1 mg/mL, or 1 mg/mL to 1.25 mg/mL.
Embodiment 33. The pharmaceutical composition of embodiment 31 or embodiment 32, wherein the pharmaceutical composition is formulated for a delivery method selected from intratumoral delivery, intravenous delivery, intramuscular delivery, peritoneal delivery, or subcutaneous delivery.
Embodiment 34. The pharmaceutical composition of any one of embodiments 31-33, further comprising a therapeutically effective amount of an antineoplastic agent.
Embodiment 35. A method of treating a subject in need thereof, the method comprising administering to a subject a therapeutically effective amount of the antibody of any one of embodiments 1-24 or the pharmaceutical composition of any one of embodiments 31-34.
Embodiment 36. The method of embodiment 35, further comprising administering a therapeutically effective amount of an antineoplastic agent to the subject.
Embodiment 37. The method of embodiment 36, wherein the effective amount of the antibody and the effective amount of the antineoplastic agent have a synergistic therapeutic effect when combined or when both are administered to a subject.
Embodiment 38. The method of any one of embodiments 35-37, wherein the subject has a tumor or a cancer, is at risk of developing the tumor or the cancer, or is suspected of having the tumor or the cancer.
Embodiment 39. The method of embodiment 38, wherein the cancer comprises a gastrointestinal cancer, a colorectal cancer, a stomach cancer, a pancreatic cancer, a neuroendocrinal cancer, or an ovarian cancer.
Embodiment 40. The method of any one of embodiments 35-39, wherein the antibody is administered to said subject at a concentration of 0.2 mg/mL to 2 mg/mL, 0.5 mg/mL to 1.5 mg/mL, 0.75 mg/mL to 1 mg/mL, or 1 mg/mL to 1.25 mg/mL.
Embodiment 41. Use of the antibody of any one of embodiments 1-24 in the manufacture of a medicament for the treatment of a tumor or cancer.
Embodiment 42. A method of selecting a subject for treatment, the method comprising: determining a risk for a neoplastic condition to progress in the subject; administering to the subject a dose of a medicament comprising the anti-CDH-17 antibody of any one of embodiments 1-24 and a drug-linker conjugate conjugated to the antibody.
Embodiment 43. The method of embodiment 42, wherein the drug-linker conjugate is selected from GGFG-monomethyl auristatin E (MMAE), GGFG-DX8951, GGFG-DXd, MC-Val-Cit-PAB-MMAE or MC-Val-Cit-PAB-DX8951.

What is claimed is:

1. An anti-cadherin-17 (CDH17) antibody comprising a light chain variable domain and a heavy chain variable domain,
wherein the light chain variable domain comprises:
a CDR L1, a CDR L2, and a CDR L3 as set forth in Table 1; and
wherein the heavy chain variable domain comprises:
a CDR H1, a CDR H2, and a CDR H3 as set forth in Table 1.

2. The antibody of claim 1, wherein the light chain variable domain comprises an amino acid sequence with at least 80%, 85%, 90%, 91%, 92%, 93%, 94%, 95%, 96%, 97%, 98%, 99%, or 100% sequence identity with a light chain variable domain sequence as set forth in Table 1.

3. The antibody of claim 1, wherein the heavy chain variable domain comprises an amino acid sequence with at least 80%, 85%, 90%, 91%, 92%, 93%, 94%, 95%, 96%, 97%, 98%, 99%, or 100% sequence identity with heavy chain variable domain sequence as set forth in Table 1.

4. The antibody of claim 1, wherein the antibody is a humanized antibody.

5. The antibody of claim 1, wherein the antibody is a chimeric antibody.

6. The antibody of claim 1, wherein the antibody is an immunoglobulin G (IgG).

7. The antibody of claim 6, wherein the antibody is an IgG1.

8. The antibody of claim 1, wherein the antibody is a Fab′ fragment.

9. The antibody of claim 1, wherein the antibody is a single chain antibody (scFv).

10. The antibody of claim 1, wherein the light chain variable domain and said heavy chain variable domain form part of a scFv.

11. The antibody of claim 1, wherein the antibody is capable of binding cadherin-17 (CDH17).

12. The antibody of claim 1, wherein the antibody is bound to CDH17.

13. The antibody of claim 12, wherein the CDH17 forms part of a cell.

14. The antibody of claim 13, wherein the cell is a cancer cell.

15. The antibody of claim 14, wherein the cancer cell is a gastrointestinal cancer cell, a colorectal cancer cell, a stomach cancer cell, a pancreas cancer cell, a neuroendocrine cancer cell, or an ovarian cancer cell.

16. An anti-cadherin-17 (CDH17) antibody, wherein the anti-CDH17 antibody binds the same epitope as an antibody comprising: a light chain variable region domain comprising a CDR L1, a CDR L2, and a CDR L3 as set forth in Table 1 and a heavy chain variable domain comprising a CDR H1, a CDR H2, and a CDR H3 as set forth in Table 1.

17. The anti-CDH17 antibody of claim 16, wherein the light chain variable domain comprises an amino acid sequence with at least 80%, 85%, 90%, 91%, 92%, 93%, 94%, 95%, 96%, 97%, 98%, 99%, or 100% sequence identity with a light chain variable domain sequence as set forth in Table 1.

18. The anti-CDH17 antibody of claim 16, wherein the heavy chain variable domain comprises an amino acid sequence with at least 80%, 85%, 90%, 91%, 92%, 93%, 94%, 95%, 96%, 97%, 98%, 99%, or 100% sequence identity with a heavy chain variable domain sequence as set forth in Table 1.

19. The anti-CDH17 antibody of claim 1, wherein the antibody is conjugated to a payload.

20. The anti-CDH17 antibody of claim 19, wherein the payload is an antineoplastic agent.

21. The anti-CDH17 antibody of claim 19, wherein the payload is selected from monomethyl auristatin E (MMAE), DX8951, or DXd.

22. The anti-CDH17 antibody of claim 1, wherein the antibody is conjugated to a linker molecule.

23. The anti-CDH17 antibody of claim 22, wherein the linker is selected from MC-Val-Cit-PAB or a tetrapeptide.

24. The anti-CDH17 antibody of claim 23, wherein the tetrapeptide is GGFG.

25. A kit comprising: the anti-CDH17 antibody of claim 1 and instructions for use in reducing the viability of a cancer cell or a tumor cell.

26. An isolated nucleic acid encoding the antibody of claim 1.

27. The isolated nucleic acid of claim 26, wherein the antibody comprises a light chain variable domain sequence and a heavy chain variable domain sequence,
wherein the light chain variable domain sequence comprises:
a CDR L1, a CDR L2, and a CDR L3 as set forth in Table 1; and
wherein the heavy chain variable domain sequence comprises:
a CDR H1, a CDR H2, and a CDR H3 as set forth in Table 1.

28. The isolated nucleic acid of claim 27, wherein the light chain variable domain sequence is encoded by a nucleic acid sequence with at least 80%, 85%, 90%, 91%, 92%, 93%, 94%, 95%, 96%, 97%, 98%, 99%, or 100% sequence identity a light chain variable nucleotide sequence as set forth in Table 1.

29. The isolated nucleic acid of claim 27, wherein the heavy chain variable domain is encoded by a nucleic acid sequence with at least 80%, 85%, 90%, 91%, 92%, 93%, 94%, 95%, 96%, 97%, 98%, 99%, or 100% sequence identity with a heavy chain variable nucleotide sequence as set forth in Table 1.

30. A cell comprising the isolated nucleic acid of claim 26.

31. A pharmaceutical composition comprising a therapeutically effective amount of the antibody of claim 1 and a pharmaceutically acceptable excipient.

32. The pharmaceutical composition of claim 31, wherein the antibody is formulated in an aqueous solution at a concentration of 0.2 mg/mL to 2 mg/mL, 0.5 mg/mL to 1.5 mg/mL, 0.75 mg/mL to 1 mg/mL, or 1 mg/mL to 1.25 mg/mL.

33. The pharmaceutical composition of claim 31, wherein the pharmaceutical composition is formulated for a delivery method selected from intratumoral delivery, intravenous delivery, intramuscular delivery, peritoneal delivery, or subcutaneous delivery.

34. The pharmaceutical composition of claim 31, further comprising a therapeutically effective amount of an antineoplastic agent.

35. A method of treating a subject in need thereof, the method comprising administering to a subject a therapeutically effective amount of the antibody of claim 1.

36. The method of claim 35, further comprising administering a therapeutically effective amount of an antineoplastic agent to the subject.

37. The method of claim 36, wherein the effective amount of the antibody and the effective amount of the antineoplastic agent have a synergistic therapeutic effect when combined or when both are administered to a subject.

38. The method of claim 35, wherein the subject has a tumor or a cancer, is at risk of developing the tumor or the cancer, or is suspected of having the tumor or the cancer.

39. The method of claim 38, wherein the cancer comprises a gastrointestinal cancer, a colorectal cancer, a stomach cancer, a pancreatic cancer, a neuroendocrinal cancer, or an ovarian cancer.

40. The method of claim 35, wherein the antibody is administered to said subject at a concentration of 0.2 mg/mL to 2 mg/mL, 0.5 mg/mL to 1.5 mg/mL, 0.75 mg/mL to 1 mg/mL, or 1 mg/mL to 1.25 mg/mL.

41. Use of the antibody of claim 1 in the manufacture of a medicament for the treatment of a tumor or cancer.

42. A method of selecting a subject for treatment, the method comprising:
determining a risk for a neoplastic condition to progress in the subject;
administering to the subject a dose of a medicament comprising the anti-CDH-17 antibody of claim 1 and a drug-linker conjugate conjugated to the antibody.

43. The method of claim 42, wherein the drug-linker conjugate is selected from GGFG-monomethyl auristatin E (MMAE), GGFG-DX8951, GGFG-DXd, MC-Val-Cit-PAB-MMAE or MC-Val-Cit-PAB-DX8951.
